SEQUENCE LISTING (31 sequences)

SEQ ID NO: 1 - protein, 37 aa, Artificial Sequence
  LL-37 peptide
LLGDFFRKSKEKIGKEFKRIVQRIKDFLRNLVPRTES

SEQ ID NO: 2 - protein, 12 aa, Artificial Sequence
  Omiganan peptide
ILRWPWWPWRRK

SEQ ID NO: 3 - protein, 12 aa, Artificial Sequence
  11B32CN peptide
KRKWPWWPWRLI

SEQ ID NO: 4 - protein, 12 aa, Artificial Sequence
  11B36CN peptide
ILKWVWWVWRRK

SEQ ID NO: 5 - protein, 13 aa, Artificial Sequence
  11E3CN peptide
ILKKWPWWPWRRK

SEQ ID NO: 6 - protein, 12 aa, Artificial Sequence
  11F4CN peptide
ILRWVWWVWRRK

SEQ ID NO: 7 - protein, 13 aa, Artificial Sequence
  11F5CN peptide
ILRRWVWWVWRRK

SEQ ID NO: 8 - protein, 11 aa, Artificial Sequence
  11F12CN peptide
RLWVWWVWRRK

SEQ ID NO: 9 - protein, 10 aa, Artificial Sequence
  11F17CN peptide
RLWVWWVWRR

SEQ ID NO: 10 - protein, 13 aa, Artificial Sequence
  11F50CN peptide
RLGGGWVWWVWRR

SEQ ID NO: 11 - protein, 10 aa, Artificial Sequence
  11F56CN peptide
RLWWVVWWRR

SEQ ID NO: 12 - protein, 10 aa, Artificial Sequence
  11F63CN peptide
RLVVWWVVRR

SEQ ID NO: 13 - protein, 10 aa, Artificial Sequence
  11F64CN peptide
RLFVWWVFRR

SEQ ID NO: 14 - protein, 10 aa, Artificial Sequence
  11F66CN peptide
RLVVWVVWRR

SEQ ID NO: 15 - protein, 10 aa, Artificial Sequence
  11F67CN peptide
RLWVWWVWRR

SEQ ID NO: 16 - protein, 10 aa, Artificial Sequence
  11F68CN peptide
RLWVWWVWRR

SEQ ID NO: 17 - protein, 10 aa, Artificial Sequence
  11F93CN peptide
WVRLWWRRVW

SEQ ID NO: 18 - protein, 9 aa, Artificial Sequence
  11G27CN peptide
WPWWPWRRK

SEQ ID NO: 19 - protein, 13 aa, Artificial Sequence
  11J02CN peptide
WRWWKPKWRWPKW

SEQ ID NO: 20 - protein, 13 aa, Artificial Sequence
  11J02ACN peptide
WRWWKPKWRWPKW

SEQ ID NO: 21 - protein, 13 aa, Artificial Sequence
  11J30CN peptide
WRWWKVAWRWVKW

SEQ ID NO: 22 - protein, 12 aa, Artificial Sequence
  11J36CN peptide
WRWWKVWRWVKW

SEQ ID NO: 23 - protein, 13 aa, Artificial Sequence
  11J58CN peptide
  MISC_FEATURE (2)..(2): X is Ornithine
  MISC_FEATURE (5)..(5): X is Ornithine
  MISC_FEATURE (9)..(9): X is Ornithine
  MISC_FEATURE (12)..(12): X is Ornithine
WXWWXVAWXWVXW

SEQ ID NO: 24 - protein, 13 aa, Artificial Sequence
  11J67CN peptide
  MISC_FEATURE (2)..(2): X is Ornithine
  MISC_FEATURE (5)..(5): X is Ornithine
  MISC_FEATURE (7)..(7): X is Ornithine
  MISC_FEATURE (9)..(9): X is Ornithine
  MISC_FEATURE (12)..(12): X is Ornithine
WXWWXPXWXWPXW

SEQ ID NO: 25 - protein, 13 aa, Artificial Sequence
  11J68CN peptide
  MISC_FEATURE (2)..(2): X is Diamino butyric acid
  MISC_FEATURE (5)..(5): X is Diamino butyric acid
  MISC_FEATURE (7)..(7): X is Diamino butyric acid
  MISC_FEATURE (9)..(9): X is Diamino butyric acid
  MISC_FEATURE (12)..(12): X is Diamino butyric acid
WXWWXPXWXWPXW

SEQ ID NO: 26 - protein, 12 aa, Artificial Sequence
  Nt-acryloyl-11B7CN peptide
ILRWPWWPWRRK

SEQ ID NO: 27 - protein, 12 aa, Artificial Sequence
  Nt-glucosyl-11J36CN peptide
WRWWKVWRWVKW

SEQ ID NO: 28 - protein, 13 aa, Artificial Sequence
  Nt-glucosyl-11J38CN peptide
WRWWKVVWRWVKW

SEQ ID NO: 29 - DNA, 20 bp, Artificial Sequence
  CpG-A ODN
gggggacgatcgtcgggggg

SEQ ID NO: 30 - DNA, 24 bp, Artificial Sequence
  CpG-B ODN
tcgtcgttttgtcgttttgtcgtt

SEQ ID NO: 31 - DNA, 25 bp, Artificial Sequence
  CpG-C ODN
tcgtcgtcgttcgaacgacgttgat

(19) United States
(12) Patent Application Publication
(10) Pub. No.: US 2020/0360471 A1
(43) Pub. Date: Nov. 19, 2020

(54) METHODS OF TREATING AND PREVENTING VIRAL INFECTIONS

(71) Applicant: Maruho Co., Ltd., Osaka (JP)

(72) Inventor: Gary Lee FEISS, Telford, PA (US)

(21) Appl. No.: 16/637,508

(22) PCT Filed: Aug. 8, 2018
(86) PCT No.: PCT/US2018/045843

§ 371 Date: Feb. 7, 2020

Related U.S. Application Data
(60) Provisional application No. 62/543,332, filed on Aug. 9, 2017.

(51) Int. Cl. A61K 38/17; A61P 31/20

(52) U.S. Cl. CPC A61K 38/17; A61P 31/20

(57) ABSTRACT

The present invention is directed to compositions comprising cationic peptides and methods of using such compositions to enhance an immune response in a subject. The invention is further directed to methods of treating and/or preventing viral infections and virally-induced cancers.

### REFERENCE TO AN ELECTRONIC SEQUENCE LISTING

The contents of the electronic sequence listing (030033_00046_Seq_List_ST25.txt; Size: 8,070 bytes; and Date of Creation: Jul. 30, 2020) is herein incorporated by reference in its entirety.

### BACKGROUND OF THE INVENTION

Cationic peptides were initially identified as naturally occurring, small, positively charged peptides that serve as a component of the host defense mechanism. These peptides demonstrated pleiotropic effects with regard to immune modulation. The synthetic cationic peptide omiganan has in vitro activity against a wide variety of bacteria and fungi, which is believed to be due to the disruption of the cytoplasmic membranes of microorganisms, resulting in depolarization and cell death.

Antiviral agents are used specifically for treating viral infections. Unlike antibiotics, which destroy their target pathogen, antiviral agents inhibit the development of their target pathogen. For example, an antiviral agent can interfere with the ability of a virus to infiltrate a target cell, can target the synthesis of new viral components once a virus enters the cell, can target the assembly of viral components into a complete virus, or can block the release of newly formed viral particles from a host cell. Because such agents target a specific viral process, they are typically effective against a specific virus, or a few closely related viruses. Accordingly, there remains a need for antiviral treatments that can be used to treat a broad range of viral infections, e.g., by stimulating a body's immune system to target the viruses. HPV is the most frequent sexually transmitted viral infection. Anogenital warts is a human papillomavirus (HPV)-induced non-malignant disorder affecting the anogenital epithelia in males and females. Clinical symptoms of HPV-induced anogenital warts include pruritus, and, inter alia, burning. Often patients also have psychosocial problems associated with the disorder. Anogenital warts are highly infectious. Approximately 65% of individuals with an infected partner develop anogenital warts. Standard treatments are topical application of podophyllotoxin (e.g., Condyline®), imiquimod (e.g., Aldara®) or sinecatechines (e.g., Veregen®) or surgical treatments like cryotherapy, local excision or laser treatment. With these drug treatments local irritation which can be treatment limiting is common, and surgical interventions also have the associated discomfort.

Even after treatment, recurrence rates of anogenital warts are reported to be as high as 30-50%. Therefore, there remains an unmet medical need for a well-tolerated topical treatment that patients can safely apply at home and a treatment which is effective in treating external anogenital warts.

HPV is also a pathogen causing the majority of cervical cancer cases (Woodman C B, Collins S I, Young L S (2007) The natural history of cervical HPV infection: unresolved issues. Nat Rev Cancer 7: 11-22). It is reported that, annually, 500,000 women are diagnosed with cervical cancer around the world, and 250,000 women die from cervical cancer (National Cancer Institute (2007) Women's Health Report, Fiscal Years 2005-2006. NCI Women's Health Report FY2005-2006). In particular, HPV16 and HPV18 are known to cause 70% of all cervical cancer cases, and thus are recognized as the most important targets in prevention of cervical cancer (Clifford G, Franceschi S, Diaz M, Munoz N, Villa L L (2006) Chapter 3: HPV type-distribution in women with and without cervical neoplastic diseases. Vaccine 24 Suppl 3: S3/26-34). There remains an unmet medical need for a well-tolerated treatment for HPV and other virally-induced cancers.

### SUMMARY OF THE INVENTION

The present invention provides a method of modulating an immune response in a subject. As disclosed herein, cationic peptides provide a novel therapeutic or prophylactic approach for the treatment of viral infection or viral inflammation. This therapeutic or prophylactic approach stimulates a subject's immune system to attack invading viruses and may be mediated by Toll Like Receptor (TLR)3, TLR7, TLR9 and RIG-1/Mda5 signaling and induced interferon responses. Accordingly, one aspect of the present invention provides a method for enhancing an immune response in a subject by administering a therapeutically effective amount of cationic peptide to the subject. The cationic peptide may be omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan or LL-37. In some embodiments, the cationic peptide is omiganan. In some embodiments, the cationic peptide is LL-37. In some embodiments, the immune response is selected from the group consisting of an interferon response, a TLR3-mediated response, a TLR7-mediated response, a RIG-1/Mda-5-mediated response, and a TLR9-mediated response. In some embodiments, the immune response is an interferon response. In other embodiments, the immune response is TLR3/RIG-1/Mda5-mediated. In yet other embodiments, the immune response is TLR7-mediated. In yet other embodiments, the immune response is TLR9-mediated. In some embodiments, the TLR9-mediated immune response is induced by a viral CpG nucleotide sequence. In some embodiments, the CpG sequence is CpG-A, CpG-B or CpG-C. In some embodiments, the immune response is an IFNα response.

Another aspect of the present invention provides a method for modulating an inflammatory response in a subject by administering a therapeutically effective amount of a cationic peptide to the subject. In some embodiments, the invention provides a method for modulating an inflammatory response in a subject comprising administering to the subject a therapeutically effective amount of a cationic peptide, wherein the therapeutically effective amount is sufficient to deliver a cationic peptide to the site of inflammatory response at a concentration of 5-25 μg/mL. In some embodiments, the invention provides a method for reducing an inflammatory response in a subject comprising administering to the subject a therapeutically effective amount of a cationic peptide, wherein the therapeutically effective amount is sufficient to deliver a cationic peptide to the site of inflammatory response at a concentration of 5-25 μg/mL. In some embodiments, the inflammatory response is induced by virus. Thus, in some embodiments, the invention provides a method for modulating a viral inflammatory response in a subject in need thereof, said method comprising administering a therapeutically effective amount of a cationic peptide to the subject. In some embodiments, the inflammatory response is caused by another antiviral agent, such as imiquimod, a TLR7 agonist used itself as an antiviral agent. In some embodiments, the cationic peptide may be omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan or LL-37. In some embodiments, the cationic peptide is omiganan. In some embodiments, the cationic peptide is LL-37. In some embodiments, the administration of the cationic peptide induces the release of cytokines, including but not limited to TNFα, IL-6, or IL-8. In some embodiments, the cytokine is IL-8. In some embodiments, the cytokine release is TLR7-mediated. In other embodiments, the cytokine release is TLR3-mediated. In yet other embodiments, the release of IL-8 is TLR3-mediated. In yet other embodiments, the release of IL-8 is TLR7-mediated.

Another aspect of the present invention provides a method for treating viral infection in a subject by administering a therapeutically effective amount of a cationic peptide to the subject, wherein the therapeutically effective amount is sufficient to provide a concentration of 5-25 μg/mL of the cationic peptide at the site of the viral infection. In some embodiments, the cationic peptide may be omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan or LL-37. In some embodiments, the cationic peptide is omiganan. In some embodiments, the cationic peptide is LL-37. In some embodiments, the plasma or local concentration of the cationic peptide is higher than the half minimal (50%) inhibitory concentration (IC) or IC₅₀ of the virus causing the viral infection 12 hours following administration of the cationic peptide to the subject. In some embodiments, the cationic peptide is administered in conjunction with a viral polynucleotide. In some embodiments, the viral polynucleotide is complexed with the cationic peptide. In some embodiments, the viral polynucleotide is a viral DNA. In some embodiments, the viral DNA is a sequence comprising CpG dinucleotides (CpG motif). In some embodiments, the cationic peptide is complexed with a synthetic CpG oligodeoxynucleotide (CpG-ODN). The CpG-ODN may be CpG-A, CpG-B, or CpG-C.

Another aspect of the present invention provides a method for preventing a viral infection in a subject by administering a prophylactically effective amount of a cationic peptide to the subject. In some embodiments, the cationic peptide may be omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan or LL-37. In some embodiments, the cationic peptide is omiganan. In some embodiments, the cationic peptide is LL-37. In some embodiments, the plasma or local concentration of the cationic peptide is higher than the half minimal (50%) inhibitory concentration (IC) or IC₅₀ of the virus 12 hours following administration of the cationic peptide to the subject. In some embodiments, the cationic peptide is administered in conjunction with a viral polynucleotide. In some embodiments, the viral polynucleotide is complexed with the cationic peptide. In some embodiments, the viral polynucleotide is a viral DNA. In some embodiments, the viral DNA is a sequence comprising CpG dinucleotides (CpG motif). In some embodiments, the viral infection is an HPV infection. In some embodiments, the HPV infection causes anogenital warts. In some embodiments, the HPV is HPV6 or HPV11. In some embodiments, the cationic peptide is complexed with a synthetic CpG-ODN. The synthetic CpG-ODN may be a CpG-A ODN, CpG-B ODN, or CpG-C ODN.

In any of the aspects described herein, the viral infection may be caused by any virus. In some embodiments, the viral infection is caused by a DNA virus. In some embodiments, the viral infection is caused by an RNA virus. Non-limiting examples of viruses include coronavirus (including, but not limited to, SARS coronavirus), coxsackievirus, cytomegalovirus, echovirus, enterovirus, Epstein-Barr virus (EBV), influenza virus, hepatotropic viruses (including, but not limited to, Hepatitis A, Hepatitis B, Hepatitis C, Hepatitis D, and Hepatitis E,), human immunodeficiency virus (HIV), human papillomavirus (HPV), herpes simplex virus (including, but not limited to, HSV-1 and HSV-2), poxvirus, norovirus, rabies virus, rhinovirus, rotavirus, Rous sarcoma virus (RSV), Varicella zoster virus, parvovirus or West Nile virus. In some embodiments, the viral infection is caused by HPV. In some embodiments, the HPV is HPVS. In some embodiments, the HPV infection causes anogenital warts. In some embodiments, the HPV is HPV6 or HPV11.

Another aspect of the present invention provides a method for treating or alleviating the symptoms of HPV-induced anogenital warts in a subject in need thereof, comprising administering to the subject a therapeutically effective amount of a cationic peptide to the site of the anogenital warts. In some embodiments, the HPV is HPV6 or HPV11. In some embodiments, the administration of the cationic peptide reduces one or more of wart count, wart height, and wart volume. In some embodiments, the administration of the cationic peptide reduces viral load at the site of the anogenital warts. In some embodiments, the cationic peptide may be omiganan, LL-37, 11B32CN, 11B36CN, 11E3CN, 11F4CN, 11F5CN, 11F12CN, 11F17CN, 11FSOCN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan. In some embodiments, the cationic peptide is omiganan or LL-37. In some embodiments, the cationic peptide is LL-37. In some embodiments, the plasma or local concentration of the cationic peptide is higher than the half minimal (50%) inhibitory concentration (IC) or IC₅₀ of the virus 12 hours following administration of the cationic peptide to the subject.

Another aspect of the present invention provides a method for treating a virally-induced cancer in a subject in need thereof, comprising administering to the subject a therapeutically effective amount of a cationic peptide. In some embodiments, the cationic peptide is administered in conjunction with a viral polynucleotide. In some embodiments, the viral polynucleotide is complexed with the cationic peptide. In some embodiments, the viral polynucleotide is a viral DNA. In some embodiments, the viral DNA is a sequence comprising CpG dinucleotides (CpG motif). In some embodiments, the cationic peptide is complexed with a synthetic CpG-ODN. The synthetic CpG-ODN may be a CpG-A ODN, CpG-B ODN, or CpG-C ODN. In some embodiments, the virally-induced cancer is caused by HPV, EBV, hepatitis B virus (HBV), hepatitis C virus (HCV), Human T-lymphotropic virus 1 (HTLV 1), Kaposi's sarcoma-associated herpesvirus (KSHV or HHV8), Merkel cell polyoma virus (MCV), or Human cytomegalovirus (CMV or HHV-5). In some embodiments, the cationic peptide may be omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan or LL-37. In some embodiments, the cationic peptide is omiganan. In some embodiments, the cationic peptide is LL-37. In some embodiments, the plasma or local concentration of the cationic peptide is higher than the half minimal (50%) inhibitory concentration (IC) or IC₅₀ of the virus 12 hours following administration of the cationic peptide to the subject.

Another aspect of the present invention provides a cationic peptide complex comprising a cationic peptide and a viral polynucleotide sequence. In some embodiments, the cationic peptide is selected from the group consisting of: omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN, and Nt-glucosyl-11J38CN. In some embodiments, the cationic peptide is omiganan or LL-37. In some embodiments, the cationic peptide is omiganan. In some embodiments, the cationic peptide is LL-37. In some embodiments, the viral polynucleotide in the complex is a viral DNA. In some embodiments, the viral DNA is a sequence comprising CpG dinucleotides (CpG motif). In some embodiments, the cationic peptide is complexed with a synthetic CpG-ODN. The synthetic CpG-ODN may be a CpG-A ODN, CpG-B ODN, or CpG-C ODN.

Another aspect of the inventions provides pharmaceutical compositions comprising the cationic peptide complexes as disclosed herein.

One aspect of the invention provides the use of a cationic peptide in the preparation of a medicament for enhancing an immune response in a subject.

Another aspect of the invention provides the use of a cationic peptide in the preparation of a medicament for modulating a viral inflammatory response in a subject.

Another aspect of the invention provides the use of a cationic peptide in the preparation of a medicament for treating viral infection in a subject, wherein the medicament comprises an amount of cationic peptide sufficient to deliver the cationic peptide to the site of viral infection at a concentration of 5-25 μg/mL.

Another aspect of the invention provides the use of a cationic peptide in the preparation of a medicament for reducing viral load in a subject, wherein the medicament comprises an amount of cationic peptide sufficient to deliver the cationic peptide to the site of viral infection a concentration of 5-25 μg/mL.

Another aspect of the invention provides the use of a cationic peptide in the preparation of a medicament for preventing a viral infection in a subject, wherein the medicament comprises an amount of cationic peptide sufficient to deliver the cationic peptide to a potential site of viral infection at a concentration of 5-25 μg/mL.

Another aspect of the invention provides the use of a cationic peptide in the preparation of a medicament for reducing inflammatory response in a subject, wherein the medicament comprises an amount of cationic peptide sufficient to deliver the cationic peptide to the site of inflammatory response at a concentration of 5-25 μg/mL.

Another aspect of the invention provides the use of a cationic peptide in the preparation of a medicament for treating or alleviating the symptoms of anogenital warts caused by an HPV infection in a subject.

In one aspect, the present invention provides a cationic peptide for use in modulating a viral inflammatory response in a subject.

Another aspect of the invention provides a cationic peptide for use in treating viral infection in a subject, wherein the medicament comprises an amount of cationic peptide sufficient to deliver the cationic peptide to the site of viral infection at a concentration of 5-25 μg/mL.

Another aspect of the invention provides a cationic peptide for use in reducing viral load in a subject, wherein the medicament comprises an amount of cationic peptide sufficient to deliver the cationic peptide to the site of viral infection a concentration of 5-25 μg/mL.

Another aspect of the invention provides a cationic peptide for use in preventing a viral infection in a subject, wherein the medicament comprises an amount of cationic peptide sufficient to deliver the cationic peptide to a potential site of viral infection at a concentration of 5-25 μg/mL.

Another aspect of the invention provides a cationic peptide for use in reducing inflammatory response in a subject, wherein the medicament comprises an amount of cationic peptide sufficient to deliver the cationic peptide to the site of inflammatory response at a concentration of 5-25 μg/mL.

Another aspect of the invention provides a cationic peptide for use in treating or alleviating the symptoms of anogenital warts caused by an HPV infection in a subject.

Another aspect of the invention provides a cationic peptide for treating a virally-induced cancer in a subject.

Another aspect of the invention provides the use of the cationic peptide as disclosed herein for preparing a medicament for use in the various methods disclosed herein. Yet another aspect of the invention provides a cationic peptide for use in the various methods disclosed herein.

In any of the aspects described herein, the cationic peptide may be provided in conjunction with a counter anion. The counter anion may be any pharmaceutically acceptable counter anion. In some embodiments, the cationic peptide is omiganan pentahydrochloride.

In any of the aspects described herein, the cationic peptide may be administered topically or parentally. In some embodiments, the administration is epicutaneous, inhalation, intranasal, an enema, eye drops, ear drops, or through a mucous membrane. In some embodiments, parental administration is subcutaneous, intravenous, intramuscular, intraarterial, intraabdominal, intraperitoneal, intraarticular, intraocular or retrobulbar, intraaural, intrathecal, intracavitary, intracelial, intraspinal, intrapulmonary or transpulmonary, intrasynovial, or intraurethral injection or infusion.

In some embodiments of the methods disclosed herein, the cationic peptide is administered topically in a composition comprising a solvent and a viscosity-increasing agent. The solvent may be water, glycerin, propylene glycol, isopropanol, ethanol, methanol, polyethylene glycol or a combination of the foregoing. In some embodiments, the solvent may be a combination of water and glycerin. In some embodiments, the viscosity-increasing agent may be dextran, polyvinylpyrrolidone, carboxyvinylpolymer, hydroxyethyl cellulose, hydroxypropyl methylcellulose or a combination of the foregoing. In some embodiments, the viscosity-increasing agent may be hydroxyethyl cellulose. In some embodiments, the composition further comprises a buffering agent comprising a monocarboxylate or a dicarboxylate. In some embodiments, the buffering agent comprises acetate, benzoate, fumarate, lactate, malonate, succinate or tartrate. In other embodiments, the buffering agent comprises benzoate.

In some embodiments, the topically administered composition used in the methods disclosed herein has a pH from about 3 to about 8. The composition disclosed in the methods herein may further contain a humectant, such as sorbitol or glycerol. In some embodiments, the composition used in the methods disclosed herein further comprises a preservative. In some embodiments, the preservative is benzoic acid, benzyl alcohol, phenoxyethanol, methylparaben, propylparaben, or a combination the foregoing.

In some embodiments, the composition used in the methods disclosed herein further comprises an additional antiviral agent. For example, the additional antiviral agent may be amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha, cidofovir, or edoxudine. In some embodiments of any of the methods disclosed herein, the composition to be administered topically is a gel.

In some embodiments, the composition used in the methods disclosed herein further comprises an additional immune modulator. For example, the additional immune modulator may be imiquimod, polyI:C, CpG DNA, or any other viral RNA or viral DNA.

In some embodiments, the cationic peptide and the additional antiviral agent are in the same composition. In some embodiments, the cationic peptide and the additional antiviral agent are administered simultaneously to the subject with viral infection or inflammation. In some embodiments, the cationic peptide and the additional antiviral agent are in different compositions. In some embodiments, the cationic peptide and the additional antiviral agent are administered sequentially to the subject with viral infection or inflammation.

### BRIEF DESCRIPTION OF THE DRAWINGS

FIG. 1 shows the effect of polyI:C(HMW) or polyI:C(HMW)/Lyovec on IL-6, IL-1β and pan-IFNα in 24-hour PBMC cultures isolated from a first male subject.

FIG. 2 shows the effect of polyI:C(HMW) or polyI:C(HMW)/Lyovec on TNFα, IL-8, and IFINβ in 24-hour PBMC cultures isolated from a first male subject. LLOQ—Lower Limit of Quantification.

FIG. 3 shows the effect of polyI:C(HMW) or polyI:C(HMW)/Lyovec on IL-6, IL-1β and pan-IFNα in 24-hour PBMC cultures isolated from a second male subject.

FIG. 4 shows the effect of polyI:C(HMW) or polyI:C(HMW)/Lyovec on TNFα, IL-8, and IFNβ in 24-hour PBMC cultures isolated from a second male subject. LLOQ—Lower Limit of Quantification.

FIG. 5 shows the effect of polyI:C(HMW) or polyI:C(HMW)/Lyovec on IL-6, IL-1β and pan-IFNα in 24-hour PBMC cultures isolated from a third male subject.

FIG. 6 shows the effect of polyI:C(HMW) or polyI:C(HMW)/Lyovec on TNFα, IL-8, and IFNβ in 24-hour PBMC cultures isolated from a third male subject. LLOQ—Lower Limit of Quantification.

FIG. 7 shows the baseline levels of the cytokines IL-6, IL1β, TNFα and IL8 in unstimulated poly I:C(HMW)/Lyovec; and poly I:C(HMW) stimulated PBMC cultures.

FIG. 8 shows the effect of omiganan and LL-37 on pan-IFNα levels in 24-hour PBMC cultures isolated from four subjects. ULOQ—Upper Limit of Quantification.

FIG. 9 shows the effect of omiganan and LL-37 on pan-IFNβ levels in 24-hour PBMC cultures isolated from four subjects. LLOQ—Lower Limit of Quantification.

FIG. 10 shows the effect of omiganan and LL-37 on IL-6 levels in 24-hour PBMC cultures isolated from four subjects. ULOQ—Upper Limit of Quantification.

FIG. 11 shows the effect of omiganan and LL-37 on TNFα levels in 24-hour PBMC cultures isolated from four subjects. ULOQ—Upper Limit of Quantification.

FIG. 12 shows the effect of omiganan and LL-37 on IL-10 levels in 24-hour PBMC cultures isolated from four subjects. ULOQ—Upper Limit of Quantification.

FIG. 13 shows the effect of omiganan and LL-37 on IL-8 levels in 24-hour PBMC cultures isolated from four subjects. ULOQ—Upper Limit of Quantification.

FIG. 14 shows the effect of a furin inhibitor on uptake of an HPVS PsV at a 1:6000 dilution in 293TT cells. The furin inhibitor inhibits viral uptake by the 293TT cells in a dose-dependent manner with an IC₅₀ of 0.2 μM.

FIG. 15 shows the effect of omiganan on uptake of an HPVS PsV at a 1:3000 dilution in 293TT cells. Omiganan inhibits viral uptake by the 293TT cells in a dose-dependent manner with an IC₅₀ of 25.67-26.00 μM.

FIG. 16 shows the effect of omiganan on uptake of an HPVS PsV at a 1:6000 dilution in 293TT cells. Omiganan inhibits viral uptake by the 293TT cells in a dose-dependent manner with an IC₅₀ of 23.48-29.07 μM.

FIG. 17 shows a summary of the subjects and the genital wart characteristics of the two treatment groups—the group treated with omiganan 2.5% and the group treated with a vehicle control.

FIG. 18A shows a summary of the change in value of the count of the target warts over time and FIG. 18B is a graphical representation of the count of all lesions visible as a change from baseline.

FIG. 19 is a graphical representation of the proportion of baseline lesions cleared.

FIG. 20 is a graphical representation of the proportion of all lesions cleared.

FIG. 21 shows the mean size of target warts (mm) per treatment group.

FIGS. 22A and 22B show the viral load (qPCR) of HPV6 and HPV11 in swab samples. The qPCR is expressed as copies/μL and natural log transformed. CFB—Change from Baseline.

FIGS. 23A and 23B show the viral load (qPCR) of HPV6 and HPV11 in swab samples. The qPCR is expressed as copies/μL, natural log transformed and also corrected for the amount of DNA in the sample. CFB—Change from Baseline.

FIGS. 24A and 24B show the expression of IL-1β in biopsies of patients treated with omiganan 2.5% or the vehicle control. CFB—Change from Baseline

FIGS. 25A and 25B show the expression of IL-8 in biopsies of patients treated with omiganan 2.5% or the vehicle control. CFB—Change from Baseline

FIG. 26 shows photography assessments of genital warts of two subjects treated with omiganan 2.5% (a and b) and one subject treated with the vehicle control (c).

FIG. 27 shows IL-1β levels in supernatant of PBMC cultures from four subjects stimulated with imiquimod (IMQ) for 3 hours, followed by omiganan (OMN) for an additional 21 hours. LLOQ—Lower Limit of Quantification.

FIG. 28 shows IL-8 levels in supernatant of PBMC cultures from four subjects stimulated with imiquimod (IMQ) for 3 hours, followed by omiganan (OMN) for an additional 21 hours. ULOQ—Upper Limit of Quantification.

FIG. 29 shows IL-6 levels in supernatant of PBMC cultures from four subjects stimulated with imiquimod (IMQ) for 3 hours, followed by omiganan (OMN) for an additional 21 hours. LLOQ—Lower Limit of Quantification.

FIG. 30 shows TNF-α levels in supernatant of PBMC cultures from four subjects stimulated with imiquimod for 3 hours, followed by omiganan for an additional 21 hours. LLOQ—Lower Limit of Quantification.

FIGS. 31A-31F shows IFN-a levels in supernatant of PBMC cultures treated with varying doses of omiganan (OMN) (30′) followed by various TLR9 stimulations (24 hrs). LLOQ—Lower Limit of Quantification.

FIG. 32 shows tumor outgrowth after three intratumoral injections of omiganan, an LL-37 variant, or PBS in TC-1 mice. Group averages for each treatment group are shown in the panel on the top left and the individual mouse data within each treatment group is shown in the remaining panels.

FIG. 33 shows flow cytometric analysis after three intratumoral injections of omiganan, an LL-37 variant, or PBS in TC-1 mice for selected cell surface markers Ly6G (top panel) and CD11c (bottom panel). LL-37 variant=LL-22, MFI=median fluorescence intensity.

FIG. 34 shows tumor outgrowth after repeated intratumoral injections of omiganan or PBS in TC-1 mice in the presence or absence of an anti-neutrophil antibody, aLy6G.

FIG. 35 shows a confocal microscopy image of a DAPI/FAM overlay of human PBMCs incubated with FAM-omiganan. The image is shown at 100 times magnification+3 times zoom in. All the cells in the solid circle show blue DAPI nuclei staining and the lighter portions of all the cells in the dashed circle show green FAM-omiganan staining. An overlay of the DAPI/FAM-omiganan staining is shown in the bottom right quadrant. The lighter portions are green from FAM-omiganan and the darker portions are blue from DAPI nuclei staining. The blue indicates DAPI nuclei staining and the green indicates FAM-omiganan.

FIG. 36 shows an overview of the different treatments administered and dose of study drug applied per day.

FIG. 37 shows the change in erythema over time per treatment as measured by colorimetry. The order of the compounds for each treatment group refers to the sequence of administration. CFB—Change from Baseline.

FIG. 38 shows a summary graph of the least squares mean (LSM) estimates of basal flow (perfusion) as change from baseline over time by treatment. The data is presented with 95% confidence intervals as error bars.

FIG. 39 shows a summary graph of qPCR of IFNγ expression relative to ABL excluding outlier.

FIG. 40 shows a summary graph of qPCR IL-10 expression relative to ABL excluding outlier.

FIG. 41 shows a summary graph of qPCR IL-6 expression relative to ABL excluding outlier.

FIG. 42 shows a summary graph of qPCR MX1 expression relative to ABL excluding outlier.

FIG. 43 shows a summary graph of qPCR MXA expression relative to ABL excluding outlier.

FIG. 44 shows a summary graph of general infiltration in the dermis.

FIG. 45 shows a summary graph of CD4 count in the dermis.

FIG. 46 shows a summary graph of CD8 count in the dermis.

FIG. 47 shows a summary graph of CD14 count in the dermis.

FIG. 48 shows the natural log (LN) of the qPCR output vs. the study day for the various subjects categorized as HPV non-responders, partial responders, and responders (top panel) and omiganan % delta count vs. study day for the various subjects categorized as HPV non-responders, partial responders, and responders (bottom panel).

FIG. 49 shows a fitted line plot of the change in target lesion at end of treatment vs. HPV DNA load pre-treatment (top panel) and a fitted line plot of the increase in IL-8 vs. HPV DNA load pre-treatment (bottom panel).

### DETAILED DESCRIPTION OF THE INVENTION

In order that the invention described herein may be fully understood, the following detailed description is set forth.

The term “herein” means the entire application.

Unless otherwise defined herein, scientific and technical terms used in this application shall have the meanings that are commonly understood by those of ordinary skill in the art to which this invention belongs. Generally, nomenclature used in connection with, and techniques of, cell and tissue culture, molecular biology, cell biology, cancer biology, neurobiology, neurochemistry, virology, immunology, microbiology, genetics, protein and nucleic acid chemistry, chemistry, and pharmacology described herein, are those well-known and commonly used in the art. Each embodiment of the inventions described herein may be taken alone or in combination with one or more other embodiments of the inventions.

The methods and techniques of the present invention are generally performed, unless otherwise indicated, according to methods of molecular biology, cell biology, biochemistry, microarray and sequencing technology well known in the art and as described in various general and more specific references that are cited and discussed throughout this specification. See, e.g. Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); Gilbert et al., “Developmental Biology, 6th ed.”, Sinauer Associates, Inc., Sunderland, Mass. (2000).

Chemistry terms used herein are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the application, where compositions are described as having, including, or comprising, specific components, it is contemplated that compositions also may consist essentially of, or consist of, the recited components.

Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The singular forms “a,” “an,” and “the” include the plurals unless the context clearly dictates otherwise.

The term “including” is used to mean “including but not limited to”. “Including” and “including but not limited to” are used interchangeably.

The term “optional” or “optionally” means that the subsequently described circumstance may or may not occur, so that the application includes instances where the circumstance occurs and instances where it does not.

The term “or” as used herein should be understood to mean “and/or,” unless the context clearly indicates otherwise.

In order to further define the invention, the following terms and definitions are provided herein.

### Definitions

The terms “subject,” “patient,” or “individual” are used interchangeably and refer to either a human or a non-human animal. These terms include mammals, such as humans, primates, livestock animals (including bovine, porcine, etc.), companion animals (e.g., canine, feline, etc.) and rodents (e.g., mice and rats).

As used herein, the term “treating,” “treat” or treatment” includes reversing, reducing, or arresting the symptoms, clinical signs or underlying pathology of a condition in a manner to improve, or stabilize the subject's condition. As used herein, and as well understood in the art, “treatment” is an approach for obtaining beneficial or desired results, including clinical results. Beneficial or desired results include, but are not limited to, prevention, alleviation, amelioration, or slowing the progression of one or more symptoms or conditions associated with a condition, diminishment of extent of disease, stabilized state of disease, delay or slowing of disease progression, amelioration or palliation of disease state, and remission (partial or total), whether detectable or undetectable. “Treatment” can also mean prolonging survival as compared to expected survival if not receiving treatment.

As used herein, the term “therapeutically effective amount” refers to an amount of a cationic peptide or composition comprising a cationic peptide that when administered to a subject will have the intended therapeutic effect (e.g. treatment, prevention or reduction of a symptom or symptoms), including but not limited to enhancing an immune response, reducing a viral inflammatory response, treating viral infection, etc. The full therapeutic effect does not necessarily occur by administration of one dose, and may occur only after administration of a series of doses. The particular “therapeutically effective dosage or amount” will depend upon e.g., the age, weight and medical condition of the subject, as well as on the method of administration and the therapeutic or combination of therapeutics selected for administration. Suitable doses are readily determined by persons skilled in the art.

As used herein, the term “prophylactically effective amount” refers to an amount of a cationic peptide or composition comprising a cationic peptide that when administered to a subject will have the intended prophylactic effect, e.g., preventing or delaying the onset (or recurrence) of a condition, disease, pathology or their symptoms. The full prophylactic effect does not necessarily occur by administration of one dose, and may occur only after administration of a series of doses. The particular “prophylactic effective dosage or amount” will depend upon e.g., the age, weight and medical condition of the subject, as well as on the method of administration and the prophylactic agent or combination of prophylactic agents selected for administration. Suitable doses are readily determined by persons skilled in the art.

As used herein, the term “IC₅₀” refers to the concentration of the cationic peptide that is required for 50% inhibition of an activity, a biological process, or a component of a process. For example, the activity includes, but is not limited to the reduction of an inflammatory response or antiviral activity.

As used herein, the term “inflammatory response” refers to a localized protective response elicited in the body of a subject, such as a mammal, when tissues are injured, e.g., by bacteria, virus, trauma, toxins, or heat. The earliest stage of an inflammatory response involves the release of cytokines such as TNFα at the site of injury which induces inflammation as a consequence of increased blood flow and capillary permeability, the influx of phagocytic cells, and tissue damage. The four typical signs of the inflammatory response are erythema (redness), heat, swelling, and pain. Another feature of inflammation is the presence of immune cells, largely mononuclear phagocytes, which are attracted to the injured area by cytokines. Neutrophils are one of the earliest types of phagocytic cells that enter a site of injury, and are classic markers of the inflammatory response. In some embodiments, the inflammatory response is in response to a viral infection.

As used herein, the term “immune response” refers to the mechanism by which the body of a subject protects itself against potentially foreign and harmful substances by recognizing and responding to antigens. Non-limiting examples of antigens include the surface of cells, viruses, fungi, or bacteria, as well as nonliving substances, such as toxins, chemicals, agents, and foreign particles. An immune response includes stimulation and/or activation of certain white blood cells, as well as release of chemicals and proteins (e.g. interferons, cytokines, etc) into the blood. For example, the cationic peptides described herein modulate the immune system by enhancing TLR3, TLR7, TLR9 and RIG-1/Mda5 signaling and inducing an interferon response.

As used herein, the term “viral inflammation” refers to an inflammatory response elicited by a viral infection.

### General Description

In one aspect, the present invention provides a method of modulating an immune response in a subject by administering a cationic peptide. The cationic peptides described herein provide a novel therapeutic approach for the treatment of viral infection or viral inflammation. Naturally occurring cationic peptides are small, positively charged peptides that serve as a component of the host defense mechanism. These peptides demonstrate pleiotropic effects with regard to immune modulation. For example, LL-37 is an antimicrobial peptide belonging to the cathelicidin family that plays an important role as a first line of defense against bacteria and other pathogens by disintegrating (damaging and puncturing) the cell membranes of bacteria and other pathogens. Similarly, the synthetic cationic peptide omiganan has in vitro activity against a wide variety of bacteria and fungi, which is believed to be due to the disruption of the cytoplasmic membranes of microorganisms, resulting in depolarization and cell death. While omiganan and LL-37 have been demonstrated to elicit antimicrobial effects in the context of disrupting pathogen-related structures and signals, the present invention relates generally to the discovery that cationic peptides also modulate antiviral immune-responses in a host subject. Without being bound by theory, these peptides are believed to promote the delivery of exogenous viral nucleic acids to the endosomal compartment of a cell, thereby enhancing TLR3, TLR7, TLR9 and RIG-1/Mda5 signaling and inducing interferon responses. Thus, targeting TLR signaling with cationic peptides may provide a new therapeutic approach for the modulation of immune responses and inflammation and the treatment of viral infections.

The present invention is based on the observation that cationic peptides can alter innate signaling pathways associated with immune, including antiviral, responses in primary human peripheral blood mononuclear cells (PBMCs). PBMCs freshly isolated from healthy donors were stimulated with ligands (e.g., PolyI:C, imiquimod etc) a) inducing specific innate signaling pathways associated with antiviral responses, and b) acting on receptors that are expressed by Langerhans cells (dermal dendritic cells) and/or keratinocytes. The ligand PolyI:C triggers the TLR3/RIG-1/Mda5 pathway, while the ligand imiquimod triggers the TLR7 pathway in PBMCs. Ligands such as CpG-A, CpG-B, and CpG-C trigger the TLR9 pathway in PBMCs. As discussed in more detail below, the cationic peptides described herein are able to modulate these signaling pathways. Accordingly, the present invention provides formulations and therapeutic uses of cationic peptides.

In one aspect, the present invention provides a method of treating or preventing virally-induced cancers. The cationic peptides disclosed herein are capable of decreasing tumor outgrowth in a mouse model of a virally-induced cancer. Further, it is demonstrated herein that the cationic peptides of the invention are able to skew the marker phenotype of tumor macrophages towards an anti-tumor profile, indicating that the cationic peptides of the invention have a cell-based adjuvant effect related to their anti-tumor activity. The cell-based adjuvant effect of the cationic peptides of the invention (e.g. omiganan) is dependent on the presence of neutrophils since blocking neutrophils reduces the adjuvant effect. Thus, in some aspects, the present invention provides a cationic peptide based adjuvant cell therapy for virally-induced cancers.

In one aspect, the present invention provides therapeutic vaccines for virally-induced cancers. The cationic peptides of the invention are capable of entering human PBMCs and exerting their immunological effects by serving as a carrier molecule for immune triggers such as viral polynucleotide sequences.

### A. Methods of Modulating An Immune Response

The present invention provides methods of modulating an immune response in a subject. For example, the cationic peptides described herein provide a novel method for the treatment of viral infection or viral inflammation. This method of treatment may be mediated by TLR3, TLR7, TLR9, RIG-1/Mda5 signaling and/or type 1 (α and β) interferon responses, which in turn activate molecules which interfere with viral replication of RNA/DNA. The interferon response may be useful in the general defense against viral infections. In some embodiments, the interferon response may be useful in the treatment of viral infections such as hepatitis B and C infections. In some embodiments, the interferon response may be useful in the treatment of an influenza virus infection. In some embodiments, the interferon response may be useful in the treatment of an HPV infection. Accordingly, a first aspect of the present invention provides a method for enhancing an immune response in a subject by administering a therapeutically effective amount of a cationic peptide to the subject. The cationic peptide may be omiganan, LL-37, 11B32CN, 11B36CN , 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is conjugated to a fatty acid. In some embodiments, the cationic peptide is omiganan. In some embodiments, the cationic peptide is LL-37. In some embodiments, the immune response is an interferon response. In some embodiments, the immune response is TLR3-mediated. In other embodiments, the immune response is TLR7-mediated. In other embodiments, the immune response is TLR9-mediated. In some embodiments, ligands such as CpG-A, CpG-B, and CpG-C trigger the TLR9 pathway in PBMCs. In other embodiments, the immune response is mediated by RIG-1/Mda5.

In some embodiments, the cationic peptide is administered to the subject for the treatment of a viral infection. In some embodiments, the cationic peptide promotes an immune response in the subject by enhancing TLR3 signaling. In some embodiments, the cationic peptide promotes an immune response in the subject by enhancing TLR7 signaling. In other embodiments, the cationic peptide promotes an immune response in the subject by enhancing TLR9 signaling. In some embodiments, ligands such as CpG-A, CpG-B, and CpG-C trigger the TLR9 pathway in PBMCs. In yet other embodiments, the cationic peptide promotes an immune response in the subject by enhancing RIG-1/Mda5 signaling. In other embodiments, the cationic peptide promotes an immune response in the subject by inducing interferon responses.

In some embodiments, the cationic peptide promotes an immune response in the subject by enhancing TLR3 and TLR7 signaling. In other embodiments, the cationic peptide promotes an immune response in the subject by enhancing TLR3 and TLR9 signaling. In some embodiments, the cationic peptide promotes an immune response in the subject by enhancing TLR3 and RIG-1/Mda5 signaling. In yet other embodiments, the cationic peptide promotes an immune response in the subject by enhancing TLR3 signaling and inducing interferon responses. In some embodiments, the cationic peptide promotes an immune response in the subject by enhancing TLR7 and TLR9 signaling. In some embodiments, the cationic peptide promotes an immune response in the subject by enhancing TLR7 and RIG-1/Mda5 signaling. In some embodiments, the cationic peptide promotes an immune response in the subject by enhancing TLR7 signaling and inducing interferon responses. In some embodiments, the cationic peptide promotes an immune response in the subject by enhancing TLR9 and RIG-1/Mda5 signaling. In some embodiments, the cationic peptide promotes an immune response in the subject by enhancing TLR9 signaling and inducing interferon responses. In other embodiments, the cationic peptide promotes an immune response in the subject by enhancing RIG-1/Mda5 signaling and inducing interferon responses.

In some embodiments, the cationic peptide promotes an immune response in the subject by enhancing TLR3, TLR7 and TLR9 signaling. In other embodiments, the cationic peptide promotes an immune response in the subject by enhancing TLR3, TLR7 and RIG-1/Mda5 signaling. In some embodiments, the cationic peptide promotes an immune response in the subject by enhancing TLR3 signaling, TLR7 signaling and inducing interferon responses. In other embodiments, the cationic peptide promotes an immune response in the subject by enhancing TLR3, TLR9 and RIG-1/Mda5 signaling. In some embodiments, the cationic peptide promotes an immune response in the subject by enhancing TLR3 signaling, TLR9 signaling and inducing interferon responses. In other embodiments, the cationic peptide promotes an immune response in the subject by enhancing TLR7, TLR9 and RIG-1/Mda5 signaling. In some embodiments, the cationic peptide promotes an immune response in the subject by enhancing TLR7 signaling, TLR9 signaling and inducing interferon responses. In some embodiments, the cationic peptide promotes an immune response in the subject by enhancing TLR3 signaling, RIG-1/Mda5 signaling and inducing interferon responses. In other embodiments, the cationic peptide promotes an immune response in the subject by enhancing TLR7 signaling, RIG-1/Mda5 signaling and inducing interferon responses. In some embodiments, the cationic peptide promotes an immune response in the subject by enhancing TLR9 signaling, RIG-1/Mda5 signaling and inducing interferon responses.

In some embodiments, the present invention provides a cationic peptide for the manufacture of a medicament for modulating an immune response. In some embodiments, the present invention provides the use of a cationic peptide for the modulating an immune response as disclosed herein.

### B. Methods of Treating a Viral Infection

Another aspect of the present invention provides a method for treating a viral infection in a subject in need thereof by administering a therapeutically effective amount of a cationic peptide to the subject.

In some embodiments, the cationic peptide is administered to the subject for the treatment of a viral infection. In some embodiments, the cationic peptide treats the viral infection in the subject by modulating TLR3 signaling. In other embodiments, the cationic peptide treats the viral infection in the subject by modulating TLR7 signaling. In some embodiments, the cationic peptide treats the viral infection in the subject by modulating TLR9 signaling. In some embodiments, the cationic peptide treats the viral infection in the subject by modulating RIG-1/Mda5 signaling. In other embodiments, the cationic peptide treats the viral infection in the subject by modulating interferon responses. In some embodiments, the cationic peptide treats the viral infection in the subject by modulating cytokine release.

In some embodiments, the cationic peptide treats the viral infection in the subject by modulating TLR3 and TLR7 signaling. In other embodiments, the cationic peptide treats the viral infection in the subject by modulating TLR3 and TLR9 signaling. In some embodiments, the cationic peptide treats the viral infection in the subject by modulating TLR3 and RIG-1/Mda5 signaling. In other embodiments, the cationic peptide treats the viral infection in the subject by modulating TLR3 signaling and interferon responses. In some embodiments, the cationic peptide treats the viral infection in the subject by modulating TLR7 and TLR9 signaling. In other embodiments, the cationic peptide treats the viral infection in the subject by modulating TLR7 and RIG-1/Mda5 signaling. In some embodiments, the cationic peptide treats the viral infection in the subject by modulating TLR7 signaling and interferon responses. In some embodiments, the cationic peptide treats the viral infection in the subject by modulating TLR9 and RIG-1/Mda5 signaling. In some embodiments, the cationic peptide treats the viral infection in the subject by modulating TLR9 signaling and interferon responses. In other embodiments, the cationic peptide treats the viral infection in the subject by modulating RIG-1/Mda5 signaling and interferon responses.

In some embodiments, the cationic peptide treats the viral infection in the subject by modulating TLR3, TLR7 and TLR9 signaling. In other embodiments, the cationic peptide treats the viral infection in the subject by modulating TLR3, TLR7 and RIG-1/Mda5 signaling. In some embodiments, the cationic peptide treats the viral infection in the subject by modulating TLR3 signaling, TLR7 signaling and interferon responses. In other embodiments, the cationic peptide treats the viral infection in the subject by modulating TLR3, TLR9 and RIG-1/Mda5 signaling. In some embodiments, the cationic peptide treats the viral infection in the subject by modulating TLR3 signaling, TLR9 signaling and interferon responses. In other embodiments, the cationic peptide treats the viral infection in the subject by modulating TLR7, TLR9 and RIG-1/Mda5 signaling. In some embodiments, the cationic peptide treats the viral infection in the subject by modulating TLR7 signaling, TLR9 signaling and interferon responses. In some embodiments, the cationic peptide treats the viral infection in the subject by modulating TLR3 signaling, RIG-1/Mda5 signaling and interferon responses. In other embodiments, the cationic peptide treats the viral infection in the subject by modulating TLR7 signaling, RIG-1/Mda5 signaling and interferon responses. In some embodiments, the cationic peptide treats the viral infection in the subject by modulating TLR9 signaling, RIG-1/Mda5 signaling and interferon responses.

In some embodiments, the invention provides a method of reducing an inflammatory response in a subject by administering a therapeutically effective amount of a cationic peptide to the subject. In some embodiments, the inflammatory response may be induced by a virus. In other embodiments, the inflammatory response may be caused by another antiviral agent, such as imiquimod, or a TLR7 agonist used itself as an antiviral agent.

In some embodiments, the cationic peptide is administered to the subject to reduce an inflammatory response. In some embodiments, the cationic peptide reduces an inflammatory response in the subject by modulating TLR3 signaling. In other embodiments, the cationic peptide reduces an inflammatory response in the subject by modulating TLR7 signaling. In some embodiments, the cationic peptide reduces an inflammatory response in the subject by modulating TLR9 signaling. In some embodiments, the cationic peptide reduces an inflammatory response in the subject by modulating RIG-1/Mda5 signaling. In other embodiments, the cationic peptide reduces an inflammatory response in the subject by modulating interferon responses.

In some embodiments, the cationic peptide reduces an inflammatory response in the subject by modulating TLR3 and TLR7 signaling. In other embodiments, the cationic peptide reduces an inflammatory response in the subject by modulating TLR3 and TLR9 signaling. In some embodiments, the cationic peptide reduces an inflammatory response in the subject by modulating TLR3 and RIG-1/Mda5 signaling. In other embodiments, the cationic peptide reduces an inflammatory response in the subject by modulating TLR3 signaling and interferon responses. In some embodiments, the cationic peptide reduces an inflammatory response in the subject by modulating TLR7 and TLR9 signaling. In other embodiments, the cationic peptide reduces an inflammatory response in the subject by modulating TLR7 and RIG-1/Mda5 signaling. In some embodiments, the cationic peptide reduces an inflammatory response in the subject by modulating TLR7 signaling and interferon responses. In some embodiments, the cationic peptide reduces an inflammatory response in the subject by modulating TLR9 and RIG-1/Mda5 signaling. In some embodiments, the cationic peptide reduces an inflammatory response in the subject by modulating TLR9 signaling and interferon responses. In other embodiments, the cationic peptide reduces an inflammatory response in the subject by modulating RIG-1/Mda5 signaling and interferon responses.

In some embodiments, the cationic peptide reduces an inflammatory response in the subject by modulating TLR3, TLR7 and TLR9 signaling. In other embodiments, the cationic peptide reduces an inflammatory response in the subject by modulating TLR3, TLR7 and RIG-1/Mda5 signaling. In some embodiments, the cationic peptide reduces an inflammatory response in the subject by modulating TLR3 signaling, TLR7 signaling and interferon responses. In other embodiments, the cationic peptide reduces an inflammatory response in the subject by modulating TLR3, TLR9 and RIG-1/Mda5 signaling. In some embodiments, the cationic peptide reduces an inflammatory response in the subject by modulating TLR3 signaling, TLR9 signaling and interferon responses. In other embodiments, the cationic peptide reduces an inflammatory response in the subject by modulating TLR7, TLR9 and RIG-1/Mda5 signaling. In some embodiments, the cationic peptide reduces an inflammatory response in the subject by modulating TLR7 signaling, TLR9 signaling and interferon responses. In some embodiments, the cationic peptide reduces an inflammatory response in the subject by modulating TLR3 signaling, RIG-1/Mda5 signaling and interferon responses. In other embodiments, the cationic peptide reduces an inflammatory response in the subject by modulating TLR7 signaling, RIG-1/Mda5 signaling and interferon responses. In some embodiments, the cationic peptide reduces an inflammatory response in the subject by modulating TLR9 signaling, RIG-1/Mda5 signaling and interferon responses.

In some embodiments, the invention provides a method of modulating an inflammatory response (e.g. release of cytokines) in a subject by administering a therapeutically effective amount of a cationic peptide to the subject. In some embodiments, the invention provides a method of modulating a viral inflammatory response (e.g. release of cytokines) in a subject by administering a therapeutically effective amount of a cationic peptide to the subject. In some embodiments, the invention provides a method of stimulating release of cytokines in a subject by administering a therapeutically effective amount of a cationic peptide to the subject. In some embodiments, the release of cytokines has an anti-viral effect. In some embodiments, the cationic peptide stimulates release of cytokines in the subject by modulating TLR3 signaling. In other embodiments, the cationic peptide stimulates release of cytokines in the subject by modulating TLR7 signaling. In some embodiments, the cationic peptide stimulates release of cytokines in the subject by modulating TLR9 signaling. In some embodiments, the cationic peptide stimulates release of cytokines in the subject by modulating RIG-1/Mda5 signaling. In other embodiments, the cationic peptide stimulates release of cytokines in the subject by modulating interferon responses.

In some embodiments, the cationic peptide stimulates release of cytokines in the subject by modulating TLR3 and TLR7 signaling. In other embodiments, the cationic peptide stimulates release of cytokines in the subject by modulating TLR3 and TLR9 signaling. In some embodiments, the cationic stimulates release of cytokines in the subject by modulating TLR3 and RIG-1/Mda5 signaling. In other embodiments, the cationic peptide stimulates release of cytokines in the subject by modulating TLR3 signaling and interferon responses. In some embodiments, the cationic peptide stimulates release of cytokines in the subject by modulating TLR7 and TLR9 signaling. In other embodiments, the cationic peptide stimulates release of cytokines in the subject by modulating TLR7 and RIG-1/Mda5 signaling. In some embodiments, the cationic peptide stimulates release of cytokines in the subject by modulating TLR7 signaling and interferon responses. In some embodiments, the cationic peptide stimulates release of cytokines in the subject by modulating TLR9 and RIG-1/Mda5 signaling. In some embodiments, the cationic peptide stimulates release of cytokines in the subject by modulating TLR9 signaling and interferon responses. In other embodiments, the cationic peptide stimulates release of cytokines in the subject by modulating RIG-1/Mda5 signaling and interferon responses.

In some embodiments, the cationic peptide stimulates release of cytokines in the subject by modulating TLR3, TLR7 and TLR9 signaling. In other embodiments, the cationic peptide stimulates release of cytokines in the subject by modulating TLR3, TLR7 and RIG-1/Mda5 signaling. In some embodiments, the cationic peptide stimulates release of cytokines in the subject by modulating TLR3 signaling, TLR7 signaling and interferon responses. In other embodiments, the cationic peptide stimulates release of cytokines in the subject by modulating TLR3, TLR9 and RIG-1/Mda5 signaling. In some embodiments, the cationic peptide stimulates release of cytokines in the subject by modulating TLR3 signaling, TLR9 signaling and interferon responses. In other embodiments, the cationic peptide stimulates release of cytokines in the subject by modulating TLR7, TLR9 and RIG-1/Mda5 signaling. In some embodiments, the cationic peptide stimulates release of cytokines in the subject by modulating TLR7 signaling, TLR9 signaling and interferon responses. In some embodiments, the cationic peptide stimulates release of cytokines in the subject by modulating TLR3 signaling, RIG-1/Mda5 signaling and interferon responses. In other embodiments, the cationic peptide stimulates release of cytokines in the subject by modulating TLR7 signaling, RIG-1/Mda5 signaling and interferon responses. In some embodiments, the cationic peptide stimulates release of cytokines in the subject by modulating TLR9 signaling, RIG-1/Mda5 signaling and interferon responses.

In some embodiments, the cationic peptide is administered to the subject to treat a viral infection. In other embodiments, the cationic peptide is administered to the subject to reduce viral load.

In some embodiments, the cationic peptide treats a viral infection in the subject by inhibiting the release of TNFα by cells including but not limited to macrophages, lymphocytes, fibroblasts and keratinocytes. In some embodiments, the cationic peptide treats a viral infection in the subject by inhibiting the release of IL-6 by monocytes and macrophages. In some embodiments, the cationic peptide treats a viral infection in the subject by inhibiting the release of IL1β by cells including but not limited to macrophage, NK cells, monocytes, and neutrophils. In some embodiments, the cationic peptide treats a viral infection in the subject by inhibiting the release of IL-8 by macrophages and other cell types including but not limited to epithelial cells, airway smooth muscle cells and endothelial cells.

In some embodiments, the cationic peptide treats a viral infection in the subject by inhibiting the release of TNFα and IL1β. In some embodiments, the cationic peptide treats a viral infection in the subject by inhibiting the release of TNFα and IL-8. In some embodiments, the cationic peptide treats a viral infection in the subject by inhibiting the release of IL-6 and IL-8. In some embodiments, the cationic peptide treats a viral infection in the subject by inhibiting the release of IL-6 and IL1β. In some embodiments, the cationic peptide treats a viral infection in the subject by inhibiting the release of TNFα and IL-6. In some embodiments, the cationic peptide treats a viral infection in the subject by inhibiting the release of IL-8 and IL1β.

In some embodiments, the cationic peptide treats a viral infection in the subject by inhibiting the release of TNFα, IL-6 and IL-8. In some embodiments, the cationic peptide treats a viral infection in the subject by inhibiting the release of TNFα, IL-6 and IL1β. In some embodiments, the cationic peptide treats a viral infection in the subject by inhibiting the release of IL-6, IL-8 and IL1β. In some embodiments, the cationic peptide treats a viral infection in the subject by inhibiting the release of TNFα, IL-8 and IL1β.

In some embodiments, the cationic peptide is administered to the subject to reduce an inflammatory response. The cationic peptide reduces an inflammatory response in the subject by inhibiting the release of TNFα by cells including but not limited to macrophages, lymphocytes, fibroblasts and keratinocytes. In some embodiments, the cationic peptide reduces an inflammatory response in the subject by inhibiting the release of IL-6 by monocytes and macrophages. In some embodiments, the cationic peptide reduces an inflammatory response in the subject by inhibiting the release of IL1β by cells including but not limited to macrophage, NK cells, monocytes, and neutrophils. In some embodiments, the cationic peptide reduces an inflammatory response in the subject by inhibiting the release of IL-8 by macrophages and other cell types including but not limited to epithelial cells, airway smooth muscle cells and endothelial cells.

In some embodiments, the cationic peptide reduces an inflammatory response in the subject by inhibiting the release of TNFα and IL-6. In some embodiments, the cationic peptide reduces an inflammatory response in the subject by inhibiting the release of TNFα and IL-8. In some embodiments, the cationic peptide reduces an inflammatory response in the subject by inhibiting the release of TNFα and IL1β. In some embodiments, the cationic peptide reduces an inflammatory response in the subject by inhibiting the release of IL-6 and IL-8. In some embodiments, the cationic peptide reduces an inflammatory response in the subject by inhibiting the release of IL-6 and IL1β. In some embodiments, the cationic peptide reduces an inflammatory response in the subject by inhibiting the release of IL-8 and IL1β.

In some embodiments, the cationic peptide reduces an inflammatory response in the subject by inhibiting the release of TNFα, IL-6 and IL-8. In some embodiments, the cationic peptide reduces an inflammatory response in the subject by inhibiting the release of TNFα, IL-6 and IL1β. In some embodiments, the cationic peptide reduces an inflammatory response in the subject by inhibiting the release of IL-6, IL-8 and IL1β. In some embodiments, the cationic peptide reduces an inflammatory response in the subject by inhibiting the release of TNFα, IL-8 and IL1β.

In some embodiments, the cationic peptide treats a viral infection in the subject by stimulating the release of TNFα by cells including but not limited to monocytes, macrophages, lymphocytes, fibroblasts and keratinocytes. In some embodiments, the cationic peptide treats a viral infection in the subject by stimulating the release of IL-6 by monocytes and macrophages. In some embodiments, the cationic peptide treats a viral infection in the subject by stimulating the release of IL-8 by monocytes, macrophages and other cell types including but not limited to epithelial cells, airway smooth muscle cells and endothelial cells.

In some embodiments, the cationic peptide treats a viral infection in the subject by stimulating the release of TNFα and IL-8. In some embodiments, the cationic peptide treats a viral infection in the subject by stimulating the release of IL-6 and IL-8. In some embodiments, the cationic peptide treats a viral infection in the subject by stimulating the release of TNFα and IL-6. In some embodiments, the cationic peptide treats a viral infection in the subject by stimulating the release of TNFα, IL-6 and IL-8.

In some embodiments, the cationic peptide is administered to the subject to modulate an inflammatory response. In some embodiments, the cationic peptide stimulates the release of TNFα by cells including but not limited to monocytes, macrophages, lymphocytes, fibroblasts and keratinocytes. In some embodiments, the cationic peptide stimulates the release of IL-6 by monocytes and macrophages. In some embodiments, the cationic peptide stimulates the release of IL-8 by monocytes, macrophages and other cell types including but not limited to epithelial cells, airway smooth muscle cells and endothelial cells.

In some embodiments, the cationic peptide stimulates the release of TNFα and IL-6. In some embodiments, the cationic peptide stimulates the release of TNFα and IL-8. In some embodiments, the cationic stimulates the release of IL-6 and IL-8. In some embodiments, the cationic peptide stimulates the release of TNFα, IL-6 and IL-8.

In some embodiments, the present invention provides a method for treating a viral infection in a subject by administering a therapeutically effective amount of a cationic peptide to the subject, wherein the therapeutically effective amount is sufficient to provide a concentration of 5-25 μg/mL of the cationic peptide at the site of the viral infection. In some embodiments, the cationic peptide is omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan or LL-37. In some embodiments, the cationic peptide is omiganan. In some embodiments, the cationic peptide is LL-37. In some embodiments, the plasma or local concentration of the cationic peptide is higher than the half minimal (50%) inhibitory concentration (IC) or IC₅₀ of the virus causing the viral infection 12 hours following administration of the cationic peptide to the subject.

In some embodiments, the present invention provides a method for reducing an inflammatory response in a subject by administering a therapeutically effective amount of a cationic peptide to the subject, wherein the therapeutically effective amount is sufficient to provide a concentration of 5-25 μg/mL of the cationic peptide at the site of the inflammation. In some embodiments, the cationic peptide is omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan. In some embodiments, the cationic peptide is LL-37. In some embodiments, the plasma or local concentration of the cationic peptide is higher than the half minimal (50%) inhibitory concentration (IC) or IC₅₀ of the virus causing the viral infection 12 hours following administration of the cationic peptide to the subject.

In some embodiments, the present invention provides a method for stimulating the release of cytokines in a subject by administering a therapeutically effective amount of a cationic peptide to the subject, wherein the therapeutically effective amount is sufficient to provide a concentration of 5-25 μg/mL of the cationic peptide at the site of the inflammation. In some embodiments, the cationic peptide is omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan. In some embodiments, the cationic peptide is LL-37. In some embodiments, the plasma or local concentration of the cationic peptide is higher than the half minimal (50%) inhibitory concentration (IC) or IC₅₀ of the virus causing the viral infection 12 hours following administration of the cationic peptide to the subject.

Another aspect of the present invention provides a method of reducing viral load in a subject in need thereof by administering a therapeutically effective amount of a cationic peptide to the subject.

In some embodiments, the cationic peptide is administered to the subject to reduce viral load. In some embodiments, the cationic peptide reduces viral load in the subject by modulating TLR3 signaling. In other embodiments, the cationic peptide reduces viral load in the subject by modulating TLR7 signaling. In some embodiments, the cationic peptide reduces viral load in the subject by modulating TLR9 signaling. In some embodiments, the cationic peptide reduces viral load in the subject by modulating RIG-1/Mda5 signaling. In other embodiments, the cationic peptide reduces viral load in the subject by modulating interferon responses.

In some embodiments, the cationic peptide reduces viral load in the subject by modulating TLR3 and TLR7 signaling. In other embodiments, the cationic peptide reduces viral load in the subject by modulating TLR3 and TLR9 signaling. In some embodiments, the cationic peptide reduces viral load in the subject by modulating TLR3 and RIG-1/Mda5 signaling. In other embodiments, the cationic peptide reduces viral load in the subject by modulating TLR3 signaling and interferon responses. In some embodiments, the cationic peptide reduces viral load in the subject by modulating TLR7 and TLR9 signaling. In other embodiments, the cationic peptide reduces viral load in the subject by modulating TLR7 and RIG-1/Mda5 signaling. In some embodiments, the cationic peptide reduces viral load in the subject by modulating TLR7 signaling and interferon responses. In some embodiments, the cationic peptide reduces viral load in the subject by modulating TLR9 and RIG-1/Mda5 signaling. In some embodiments, the cationic peptide reduces viral load in the subject by modulating TLR9 signaling and interferon responses. In other embodiments, the cationic peptide reduces viral load in the subject by modulating RIG-1/Mda5 signaling and interferon responses.

In some embodiments, the cationic peptide reduces viral load in the subject by modulating TLR3, TLR7 and TLR9 signaling. In other embodiments, the cationic peptide reduces viral load in the subject by modulating TLR3, TLR7 and RIG-1/Mda5 signaling. In some embodiments, the cationic peptide reduces viral load in the subject by modulating TLR3 signaling, TLR7 signaling and interferon responses. In other embodiments, the cationic peptide reduces viral load in the subject by modulating TLR3, TLR9 and RIG-1/Mda5 signaling. In some embodiments, the cationic peptide reduces viral load in the subject by modulating TLR3 signaling, TLR9 signaling and interferon responses. In other embodiments, the cationic peptide reduces viral load in the subject by modulating TLR7, TLR9 and RIG-1/Mda5 signaling. In some embodiments, the cationic peptide reduces viral load in the subject by modulating TLR7 signaling, TLR9 signaling and interferon responses. In some embodiments, the cationic peptide reduces viral load in the subject by modulating TLR3 signaling, RIG-1/Mda5 signaling and interferon responses. In other embodiments, the cationic peptide reduces viral load in the subject by modulating TLR7 signaling, RIG-1/Mda5 signaling and interferon responses. In some embodiments, the cationic peptide reduces viral load in the subject by modulating TLR9 signaling, RIG-1/Mda5 signaling and interferon responses.

In some embodiments, the invention provides a method of reducing viral load in a subject by administering a therapeutically effective amount of a cationic peptide to the subject. The cationic peptide may be omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan. In some embodiments, the cationic peptide is LL-37. In some embodiments, the administration of the cationic peptide reduces the viral load by inhibiting the release of cytokines, such as TNFα, IL-6, IL1β or IL-8. In some embodiments, the cytokine release is TLR7-mediated. In some embodiments, the release of IL-8 is TLR3-mediated. In some embodiments, the administration of the cationic peptide reduces the viral load by stimulating the release of cytokines, such as TNFα, IL-6, or IL-8. In some embodiments, the cytokine release is TLR7-mediated. In some embodiments, the release of IL-8 is TLR7-mediated. In some embodiments, the cytokine release is TLR7-mediated. In some embodiments, the release of IL-8 is TLR3-mediated. In some embodiments, the cytokine release is TLR9-mediated.

In some embodiments, the cationic peptide is administered to the subject to reduce viral load. The cationic peptide reduces viral load in the subject by inhibiting the release of TNFα by cells including but not limited to macrophages, lymphocytes, fibroblasts and keratinocytes. In some embodiments, the cationic peptide reduces viral load in the subject by inhibiting the release of IL-6 by monocytes and macrophages. In some embodiments, the cationic peptide reduces viral load in the subject by inhibiting the release of IL1β by cells including but not limited to macrophage, NK cells, monocytes, and neutrophils. In some embodiments, the cationic peptide reduces viral load in the subject by inhibiting the release of IL-8 by macrophages and other cell types including but not limited to epithelial cells, airway smooth muscle cells and endothelial cells.

In some embodiments, the cationic peptide reduces viral load in the subject by inhibiting the release of TNFα and IL-6. In some embodiments, the cationic peptide reduces viral load in the subject by inhibiting the release of TNFα and IL-8. In some embodiments, the cationic peptide reduces viral load in the subject by inhibiting the release of TNFα and IL1β. In some embodiments, the cationic peptide reduces viral load in the subject by inhibiting the release of IL-6 and IL-8. In some embodiments, the cationic peptide reduces viral load in the subject by inhibiting the release of IL-6 and IL1β. In some embodiments, the cationic peptide reduces viral load in the subject by inhibiting the release of IL-8 and IL1β.

In some embodiments, the cationic peptide reduces viral load in the subject by inhibiting the release of TNFα, IL-6 and IL-8. In some embodiments, the cationic peptide reduces viral load in the subject by inhibiting the release of TNFα, IL-6 and IL1β. In some embodiments, the cationic peptide reduces viral load in the subject by inhibiting the release of IL-6, IL-8 and IL1β. In some embodiments, the cationic peptide reduces viral load in the subject by inhibiting the release of TNFα, IL-8 and IL1β.

In some embodiments, the cationic peptide is administered to the subject to reduce viral load. The cationic peptide reduces viral load in the subject by stimulating the release of TNFα by cells including but not limited to monocytes, macrophages, lymphocytes, fibroblasts and keratinocytes. In some embodiments, the cationic peptide reduces viral load in the subject by stimulating the release of IL-6 by monocytes and macrophages. In some embodiments, the cationic peptide reduces viral load in the subject by stimulating the release of IL-8 by monocytes, macrophages and other cell types including but not limited to epithelial cells, airway smooth muscle cells and endothelial cells.

In some embodiments, the cationic peptide reduces viral load in the subject by stimulating the release of TNFα and IL-6. In some embodiments, the cationic peptide reduces viral load in the subject by stimulating the release of TNFα and IL-8. In some embodiments, the cationic peptide reduces viral load in the subject by stimulating the release of IL-6 and IL-8. In some embodiments, the cationic peptide reduces viral load in the subject by stimulating the release of TNFα, IL-6 and IL-8.

The present invention provides cationic peptide complexes comprising a cationic peptide and a viral polynucleotide sequence. Cationic peptides of the invention enter human PBMCs and exert their immunological effects by serving as a carrier molecule for immune triggers such as viral polynucleotide sequences. The cationic peptide may be associated with the viral polynucleotide sequence by an electrostatic interaction or a covalent interaction. In some embodiments, the present invention provides a composition comprising cationic peptide complex. The cationic peptide complexes are useful for treating and/or preventing viral infections.

Any viral infection may be treated with the cationic peptides of the present invention. The inflammatory response caused by a viral infection may also be treated with the cationic peptides of the invention. In some embodiments, the viral infection is caused by a DNA virus. In other embodiments, the viral infection is caused by a RNA virus. Examples of viruses that may be treated with the cationic peptides of the present invention include, but are not limited to, coronavirus (including, but not limited to, SARS coronavirus), coxsackieviruses cytomegaloviruses, echoviruses, enteroviruses, Epstein-Barr virus, influenza virus, hepatotropic viruses (including but not limited to Hepatitis A, Hepatitis B, Hepatitis C, Hepatitis D, and Hepatitis E), human immunodeficiency virus (HIV), human papilloma virus (HPV), herpes simplex virus (including, but not limited to, HSV-1 and HSV-2), poxvirus, norovirus, rabies virus, rhinovirus, rotavirus, Rous sarcoma virus (RSV), Varicella zoster virus, parvovirus and West Nile virus. The conditions caused by the viral infections include, but are not limited to, acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection, gingivostomatitis, tonsillitis & pharyngitis, keratoconjunctivitis, herpes labialis, cold sores, aseptic meningitis, Cytomegalic inclusion disease, Kaposi sarcoma, AIDS, influenza, Reye syndrome, measles, post-infectious encephalomyelitis, mumps, hyperplastic epithelial lesions (common, flat, plantar and anogenital warts, laryngeal papillomas, epidermodysplasia verruciformis), giant condylomata (Buschke-Löwenstein tumors), cervical intraepithelial neoplasia, cervical carcinoma, vulvar intraepithelial neoplasia, vulvar cancer, penile carcinoma, anal intraepithelial neoplasia, anal carcinoma, actinic keratosis, squamous cell carcinomas, conjunctival papillomas and carcinomas, oral leukoplakia and carcinomas, croup, pneumonia, common cold, poliomyelitis, rabies, bronchiolitis, influenza-like syndrome, severe bronchiolitis with pneumonia, german measles, congenital rubella, varicella (chicken pox) and herpes zoster (shingles), BK virus infection, bolivian hemorrhagic fever, chikungunya, Colorado tick fever, Crimean-Congo hemorrhagic fever, cytomegalovirus infection, dengue fever, ebola hemorrhagic fever, enterovirus infection, erythema infectiosum (Fifth disease), exanthema subitum (Sixth disease), hand, foot and mouth disease, hantavirus pulmonary syndrome, heartland virus disease, human bocavirus infection, human metapneumovirus infection, human parainfluenza virus infection, lassa fever, Marburg hemorrhagic fever, Middle East respiratory syndrome, monkeypox, progressive multifocal leukoencephalopathy, Rift valley fever, Severe Acute Respiratory Syndrome (SARS), subacute sclerosing panencephalitis, Venezuelan equine encephalitis, Venezuelan hemorrhagic fever, West Nile fever and Yellow fever.

Traditional treatment of viral infections includes treatment of symptoms associated with the infection and the use of antiviral agents. In some embodiments, the method further comprises administering an additional antiviral agent. Additional antiviral agents include, without limitation, trifluridine, penciclovir, famciclovir, ganciclovir, foscarnet, acyclovir, cidofovir, valacyclovir and valganciclovir for the treatment of herpes infections; aspirin, oseltamivir, zanamivir, acetaminophen, ibuprofen for the treatment of influenza; antiretroviral agents for the treatment of HIV infections, that block HIV from entering human cells or block the activity of one of the enzymes that HIV needs to replicate inside human cells; and interferon-α, pegylated interferon-α, lamivudine, adefovir, entecavir, telbivudine and tenofovir for the treatment of chronic hepatitis.

In one aspect, the present invention provides compositions and methods for treating virally-induced skin diseases. They include, for example, skin diseases such as warts, anogenital warts, benign or malignant tumors of the skin and/or mucosa which are caused by papilloma viruses, for example verrucae plantares, verrucae vulgares, verrucae planae juveniles, epidermodysplasia verruciformis, Condylomata acuminata, Condylomata plana, bowenoid papulosis, papillomas on the larynx and oral mucosa, focal epithelial hyperplasia, herpes labialis, Kaposi's sarcoma, varicella and shingles. These viral skin diseases and/or tumor diseases are caused by at least one papilloma virus or viruses, in particular human papilloma viruses, such as HPV 1, 2, 3, 4, 5, 6, 8, 9, 11, 12, 13, 14, 15, 16, 17, 18, 19-29, 31, 32, 34, 36-38, 46-50, 56, 58, by at least one herpes virus or herpes viruses, such as herpes simplex virus 1, herpes simplex virus 2, varicella zoster virus or human herpes virus, such as 1, 2, 3, 4, 7 or 8. In some embodiments, a cationic peptide composition of the invention is administered to a subject to treat or ameliorate the symptoms associated with a virally-induced skin disease. In some embodiments, the administration of the cationic peptide reduces the viral load. In some embodiments, a cationic peptide composition of the invention is administered to a subject to treat or ameliorate the symptoms associated with an HPV-induced skin disease. HPV-induced skin diseases may include ordinary warts, plantar warts, genital warts, anal warts, or tumors of the skin and/or mucosa. In some embodiments, the tumors of the skin and/or mucosa are benign. In some embodiments, the tumors of the skin and/or mucosa are malignant. In some embodiments, the HPV-induced skin disease is non-melanoma skin cancer. In some embodiments, the cancer is squamous cell skin cancer.

In some embodiments, a cationic peptide composition of the invention is administered to a subject to treat HPV-induced anogenital warts. HPV-induced anogenital warts is a non-malignant disease affecting the anogenital epithelia. Anogenital warts can present externally (e.g., vulva, perineum, penis, scrotum, anus) or internally (e.g., cervix, vagina, urethra). The most common HPV types causing anogenital warts are HPV 6 and HPV 11, which are low risk HPV genotypes, but (co-)infections with other HPV-types can also occur. In some embodiments, the HPV is HPV6 or HPV11. In some embodiments, the cationic peptide clears the warts. In some embodiments, the cationic peptide leads to at least 25%, at least 30%, at least 40%, at least 50%, at least 60%, at least 70%, at least 80%, at least 90%, or at least 95% clearance of warts. In some embodiments, the cationic peptide completely clears the warts. In some embodiments, the cationic reduces one or more of: wart count, wart size, wart height, or wart volume. In some embodiments, the cationic peptide reduces the wart count by at least 25%, at least 30%, at least 40%, at least 50%, at least 60%, at least 70%, at least 80%, at least 90%, or at least 95%. In some embodiments, the cationic peptide reduces the wart size by at least 25%, at least 30%, at least 40%, at least 50%, at least 60%, at least 70%, at least 80%, at least 90%, or at least 95%. In some embodiments, the cationic peptide reduces the wart height by at least 25%, at least 30%, at least 40%, at least 50%, at least 60%, at least 70%, at least 80%, at least 90%, or at least 95%. In some embodiments, the cationic peptide reduces the wart volume by at least 25%, at least 30%, at least 40%, at least 50%, at least 60%, at least 70%, at least 80%, at least 90%, or at least 95%. In some embodiments, the cationic peptide reduces the viral load at the site of the wart. In some embodiments, the cationic peptide reduces the viral load by at least 25%, at least 30%, at least 40%, at least 50%, at least 60%, at least 70%, at least 80%, at least 90%, or at least 95%. In some embodiments, the reduction is measured relative to a reference level. Reference levels may be established using methods known in the art. In some embodiments, the reference level is the level (e.g. viral load, wart count, wart size, wart volume, wart height, etc.) prior to the start of treatment. In some embodiments, the cationic peptide modulates cytokine release. In some embodiments, the cationic peptide induces upregulation of IL-8.

In some embodiments, the present invention provides a cationic peptide or cationic peptide complex for the manufacture of a medicament for treating a viral infection. In some embodiments, the present invention provides the use of a cationic peptide or cationic peptide complex for treating a viral infection. In some embodiments, the present invention provides a cationic peptide or cationic peptide complex for the manufacture of a medicament for treating HPV-induced anogenital warts. In some embodiments, the present invention provides a cationic peptide or cationic peptide complex for use in treating HPV-induced anogenital warts.

### C. Methods of Preventing a Viral Infection

Another aspect of the present invention provides a method for preventing a viral infection (e.g. preventing viral uptake by a cell) in a subject in need thereof by administering the cationic peptide. In some embodiments, the cationic peptide prevents the viral infection in the subject by modulating TLR3 signaling. In other embodiments, the cationic peptide prevents the viral infection in the subject by modulating TLR7 signaling. In some embodiments, the cationic peptide prevents the viral infection in the subject by modulating TLR9 signaling. In some embodiments, the cationic peptide prevents the viral infection in the subject by modulating RIG-1/Mda5 signaling. In other embodiments, the cationic peptide prevents the viral infection in the subject by modulating interferon responses. In other embodiments, the cationic peptide prevents the viral infection in the subject by modulating cytokine release.

In some embodiments, the cationic peptide prevents the viral infection in the subject by modulating TLR3 and TLR7 signaling. In other embodiments, the cationic peptide prevents the viral infection in the subject by modulating TLR3 and TLR9 signaling. In some embodiments, the cationic peptide prevents the viral infection in the subject by modulating TLR3 and RIG-1/Mda5 signaling. In other embodiments, the cationic peptide prevents the viral infection in the subject by modulating TLR3 signaling and interferon responses. In some embodiments, the cationic peptide prevents the viral infection in the subject by modulating TLR7 and TLR9 signaling. In other embodiments, the cationic peptide prevents the viral infection in the subject by modulating TLR7 and RIG-1/Mda5 signaling. In some embodiments, the cationic peptide prevents the viral infection in the subject by modulating TLR7 signaling and interferon responses. In some embodiments, the cationic peptide prevents the viral infection in the subject by modulating TLR9 and RIG-1/Mda5 signaling. In some embodiments, the cationic peptide prevents the viral infection in the subject by modulating TLR9 signaling and interferon responses. In other embodiments, the cationic peptide prevents the viral infection in the subject by modulating RIG-1/Mda5 signaling and interferon responses.

In some embodiments, the cationic peptide prevents the viral infection in the subject by modulating TLR3, TLR7 and TLR9 signaling. In other embodiments, the cationic peptide prevents the viral infection in the subject by modulating TLR3, TLR7 and RIG-1/Mda5 signaling. In some embodiments, the cationic peptide prevents the viral infection in the subject by modulating TLR3 signaling, TLR7 signaling and interferon responses. In other embodiments, the cationic peptide prevents the viral infection in the subject by modulating TLR3, TLR9 and RIG-1/Mda5 signaling. In some embodiments, the cationic peptide prevents the viral infection in the subject by modulating TLR3 signaling, TLR9 signaling and interferon responses. In other embodiments, the cationic peptide prevents the viral infection in the subject by modulating TLR7, TLR9 and RIG-1/Mda5 signaling. In some embodiments, the cationic peptide prevents the viral infection in the subject by modulating TLR7 signaling, TLR9 signaling and interferon responses. In some embodiments, the cationic peptide prevents the viral infection in the subject by modulating TLR3 signaling, RIG-1/Mda5 signaling and interferon responses. In other embodiments, the cationic peptide prevents the viral infection in the subject by modulating TLR7 signaling, RIG-1/Mda5 signaling and interferon responses. In some embodiments, the cationic peptide prevents the viral infection in the subject by modulating TLR9 signaling, RIG-1/Mda5 signaling and interferon responses.

In some embodiments, the cationic peptide prevents the viral infection in the subject by inhibiting the release of TNFα by cells including but not limited to macrophages, lymphocytes, fibroblasts and keratinocytes. In some embodiments, the cationic peptide prevents the viral infection in the subject by inhibiting the release of IL-6 by monocytes and macrophages. In some embodiments, the cationic peptide prevents the viral infection in the subject by inhibiting the release of IL1β by cells including but not limited to macrophage, NK cells, monocytes, and neutrophils. In some embodiments, the cationic peptide prevents the viral infection in the subject by inhibiting the release of IL-8 by macrophages and other cell types including but not limited to epithelial cells, airway smooth muscle cells and endothelial cells.

In some embodiments, the cationic peptide prevents the viral infection in the subject by inhibiting the release of TNFα and IL1β. In some embodiments, the cationic peptide prevents the viral infection in the subject by inhibiting the release of TNFα and IL-8. In some embodiments, the cationic peptide prevents the viral infection in the subject by inhibiting the release of IL-6 and IL-8. In some embodiments, the cationic peptide prevents the viral infection in the subject by inhibiting the release of IL-6 and IL1β. In some embodiments, the cationic peptide prevents the viral infection in the subject by inhibiting the release of TNFα and IL-6. In some embodiments, the cationic peptide prevents the viral infection in the subject by inhibiting the release of IL-8 and IL1β.

In some embodiments, the cationic peptide prevents the viral infection in the subject by inhibiting the release of TNFα, IL-6 and IL-8. In some embodiments, the cationic peptide prevents the viral infection in the subject by inhibiting the release of TNFα, IL-6 and IL1β. In some embodiments, the cationic peptide prevents the viral infection in the subject by inhibiting the release of IL-6, IL-8 and IL1β. In some embodiments, the cationic peptide prevents the viral infection in the subject by inhibiting the release of TNFα, IL-8 and IL1β.

In some embodiments, the cationic peptide prevents the viral infection in the subject by stimulating the release of TNFα by cells including but not limited to monocytes, macrophages, lymphocytes, fibroblasts and keratinocytes. In some embodiments, the cationic peptide prevents the viral infection in the subject by stimulating the release of IL-6 by monocytes and macrophages. In some embodiments, the cationic peptide prevents the viral infection in the subject by stimulating the release of IL-8 by monocytes, macrophages and other cell types including but not limited to epithelial cells, airway smooth muscle cells and endothelial cells.

In some embodiments, the cationic peptide prevents the viral infection in the subject by stimulating the release of TNFα and IL-8. In some embodiments, the cationic peptide prevents the viral infection in the subject by stimulating the release of IL-6 and IL-8. In some embodiments, the cationic peptide prevents the viral infection in the subject by stimulating the release of TNFα and IL-6. In some embodiments, the cationic peptide prevents the viral infection in the subject by stimulating the release of TNFα, IL-6 and IL-8.

Another aspect of the present invention provides a method for preventing a viral infection (e.g. preventing viral uptake by a cell) in a subject in need thereof by administering a prophylactically effective amount of a cationic peptide to the subject. The cationic peptide may be omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan or LL-37. In some embodiments, the cationic peptide is omiganan. In some embodiments, the cationic peptide is LL-37. In some embodiments, the administration of the cationic peptide prevents the viral infection by inhibiting the release of cytokines, such as TNFα, IL-6, IL1β or IL-8. In some embodiments, the cytokine release is TLR7-mediated. In some embodiments, the release of IL-8 is TLR7-mediated. In some embodiments, the cytokine release is TLR3-mediated. In some embodiments, the release of IL-8 is TLR3-mediated. In some embodiments, the cytokine release is TLR9-mediated

Any viral infection may be prevented with the cationic peptides of the present invention. In some embodiments, the viral infection is caused by a DNA virus. In some embodiments, the viral infection is caused by a RNA virus. Examples of viruses that may be prevented with the cationic peptides of the present invention include, but are not limited to, coronavirus (including, but not limited to, SARS coronavirus), coxsackieviruses cytomegaloviruses, echoviruses, enteroviruses, Epstein-Barr virus, influenza virus, hepatotropic viruses (including but not limited to Hepatitis A, Hepatitis B, Hepatitis C, Hepatitis D, and Hepatitis E), human immunodeficiency virus (HIV), human papilloma virus (HPV), herpes simplex virus (including, but not limited to, HSV-1 and HSV-2), poxvirus, norovirus, rabies virus, rhinovirus, rotavirus, Rous sarcoma virus (RSV), Varicella zoster virus, parvovirus and West Nile virus. Infections include, but are not limited to, acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection, gingivostomatitis, tonsillitis & pharyngitis, keratoconjunctivitis, herpes labialis, cold sores, aseptic meningitis, Cytomegalic inclusion disease, Kaposi sarcoma, AIDS, influenza, Reye syndrome, measles, post-infectious encephalomyelitis, mumps, hyperplastic epithelial lesions (common, flat, plantar and anogenital warts, laryngeal papillomas, epidermodysplasia verruciformis), giant condylomata (Buschke-Löwenstein tumors), cervical intraepithelial neoplasia, cervical carcinoma, vulvar intraepithelial neoplasia, vulvar cancer, penile carcinoma, anal intraepithelial neoplasia, anal carcinoma, actinic keratosis, squamous cell carcinomas, conjunctival papillomas and carcinomas, oral leukoplakia and carcinomas, croup, pneumonia, common cold, poliomyelitis, rabies, bronchiolitis, influenza-like syndrome, severe bronchiolitis with pneumonia, german measles, congenital rubella, varicella (chicken pox) and herpes zoster (shingles), BK virus infection, bolivian hemorrhagic fever, chikungunya, Colorado tick fever, Crimean-Congo hemorrhagic fever, cytomegalovirus infection, dengue fever, ebola hemorrhagic fever, enterovirus infection, erythema infectiosum (Fifth disease), exanthema subitum (Sixth disease), hand, foot and mouth disease, hantavirus pulmonary syndrome, heartland virus disease, human bocavirus infection, human metapneumovirus infection, human parainfluenza virus infection, lassa fever, Marburg hemorrhagic fever, Middle East respiratory syndrome, monkeypox, progressive multifocal leukoencephalopathy, Rift valley fever, Severe Acute Respiratory Syndrome (SARS), subacute sclerosing panencephalitis, Venezuelan equine encephalitis, Venezuelan hemorrhagic fever, West Nile fever and Yellow fever. In some embodiments, the virus is HPV. In some embodiments, the HPV is HPVS. In other embodiments, the HPV is HPV 6. In yet other embodiments, the HPV is HPV11.

In some embodiments, the present invention provides a cationic peptide or cationic peptide complex for the manufacture of a medicament for preventing a viral infection. In some embodiments, the present invention provides a cationic peptide or cationic peptide complex for use in preventing a viral infection.

### D. Methods of Treating Virally-Induced Cancers

The compositions and methods of the present invention are useful in methods of treating or preventing virally-induced cancers. The cationic peptides are able to decrease tumor outgrowth in a mouse model of a virally-induced cancer. Further, it is demonstrated herein that the cationic peptides of the invention are able to skew the marker phenotype of tumor macrophages towards an anti-tumor profile, indicating that the cationic peptides of the invention have a cell-based adjuvant effect related to their anti-tumor activity. The cell-based adjuvant effect of omiganan is dependent on the presence of neutrophils since blocking neutrophils reduces the adjuvant effect. Thus, in some aspects, the present invention provides a cationic peptide based adjuvant cell therapy for virally-induced cancers. In some embodiments, the virally-induced cancer is caused by Human Papillomavirus (HPV), Epstein-Barr virus (EBV), hepatitis B virus (HBV), hepatitis C virus (HCV), Human T-lymphotropic virus 1 (HTLV 1), Kaposi's sarcoma-associated herpesvirus (KSHV or HHV8), Merkel cell polyoma virus (MCV), or Human cytomegalovirus (CMV or HHV-5). In some embodiments the virally-induced cancer is caused by HPV16, HPV18, HPV31, HPV33, HPV35, HPV45, HPV52, HPV58, or HPV59.

In some embodiments, the cationic peptide is administered to the subject to treat a virally-induced cancer infection. In some embodiments, the cationic peptide treats a virally-induced cancer by decreasing tumor outgrowth. In some embodiments, the cationic peptide treats a virally-induced cancer by having a neutrophil-dependent cell-based adjuvant effect. In some embodiments, the cell-based adjuvant effect includes skewing the differentiation of tumor macrophages towards an anti-tumor profile. This may include the downregulation of CD11c. In some embodiments, the cationic peptide is administered in conjunction with a viral polynucleotide. In some embodiments, the viral polynucleotide is complexed with the cationic peptide. In some embodiments, the viral polynucleotide is a viral DNA. In some embodiments, the viral DNA is a sequence comprising CpG dinucleotides (CpG motif). In some embodiments, the viral polynucleotide comprises a sequence from the L1 genomic region of HPV. In some embodiments, the cationic peptide is complexed with a synthetic CpG-ODN. The synthetic CpG-ODN may be a CpG-A ODN, CpG-B ODN, or CpG-C ODN.

In some embodiments, the present invention provides a method for treating a virally-induced cancer in a subject by administering a therapeutically effective amount of a cationic peptide to the subject, wherein the therapeutically effective amount is sufficient to provide a concentration of 5-25 μg/mL of the cationic peptide at the site of the inflammation. In some embodiments, the cationic peptide is omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan or LL-37. In some embodiments, the cationic peptide is omiganan. In some embodiments, the cationic peptide is LL-37.

In one aspect, the present invention provides therapeutic vaccines for virally-induced cancers. The cationic peptides of the invention enter human PBMCs and exert their immunological effects by serving as a carrier molecule for immune triggers such as viral polynucleotide sequences. Accordingly, the present invention provides cationic peptide complexes comprising a cationic peptide and a viral polynucleotide sequence. The cationic peptide may be associated with the viral polynucleotide sequence by an electrostatic interaction or a covalent interaction. In some embodiments, the present invention provides a composition comprising cationic peptide complex. The cationic peptide complexes are useful as therapeutic vaccines for treating and/or preventing virally-induced cancers.

In some embodiments, the present invention provides a method for treating a virally-induced cancer in a subject by administering a therapeutically effective amount of a cationic peptide complex to the subject, wherein the therapeutically effective amount is sufficient to provide a concentration of 5-25 μg/mL of the cationic peptide. In some embodiments, the cationic peptide is omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN or Nt-glucosyl-11J38CN. In some embodiments, the cationic peptide is omiganan or LL-37. In some embodiments, the cationic peptide is omiganan. In some embodiments, the cationic peptide is LL-37.

In some embodiments, the cationic peptide complexes of the present invention are useful in methods of cell therapy to treat virally-induced cancers. Autologous immune cells (such as but not limited to dendritic cells, T cells, etc.) may be primed with a cationic peptide complex of the invention prior to administration to the subject. In some embodiments, the cationic peptide complexes comprising a cationic peptide and a viral polynucleotide sequence. The cationic peptide may be associated with the viral polynucleotide sequence by an electrostatic interaction or a covalent interaction. In some embodiments, the viral polynucleotide is a viral DNA. In some embodiments, the viral DNA is a sequence comprising CpG dinucleotides (CpG motif). In some embodiments, the viral polynucleotide comprises a sequence from the L1 genomic region of HPV. In some embodiments, the cationic peptide is complexed with a synthetic CpG-ODN. The synthetic CpG-ODN may be a CpG-A ODN, CpG-B ODN, or CpG-C ODN.

In some embodiments, the present invention provides a cationic peptide or cationic peptide complex for the manufacture of a medicament for treating a virally-induced cancer. In some embodiments, the present invention provides a cationic peptide or cationic peptide complex for use in treating a virally-induced cancer.

### E. Additional Agents

In some embodiments, the methods disclosed herein further comprise administering an additional antiviral agent. Additional antiviral agents include, without limitation, trifluridine, penciclovir, famciclovir, ganciclovir, foscarnet, acyclovir, cidofovir, valacyclovir and valganciclovir for the treatment of herpes infections; aspirin, oseltamivir, zanamivir, acetaminophen, ibuprofen for the treatment of influenza; antiretroviral agents for the treatment of HIV infections, that block HIV from entering human cells or block the activity of one of the enzymes that HIV needs to replicate inside human cells; and interferon-α, pegylated interferon-α, lamivudine, adefovir, entecavir, telbivudine and tenofovir for the treatment of chronic hepatitis.

In some embodiments, the method further comprises administering an additional viral uptake inhibitor. Additional viral uptake inhibitors include, without limitation, furin inhibitor I, amantadine, rimantadine, interferon, glycyrrhizin, and pentafuside.

The present invention recognizes that the effectiveness of conventional cancer therapies (e.g., chemotherapy, radiation therapy, phototherapy, immunotherapy, and surgery) can be enhanced through the use of the subject cationic peptide compositions. Accordingly, cationic peptides may be used in combination therapies for the treatment, prevention, or management of virally-induced cancer. The cationic peptides or cationic peptide complexes may be administered to patients in combination with radiation and/or surgical treatment as well as with cytotoxic chemotherapy. Such combination treatments may work synergistically and allow reduction of dosage of each of the individual treatments, thereby reducing the detrimental side effects exerted by each treatment at higher dosages. In other instances, malignancies that are refractory to a treatment may respond to a combination therapy of two or more different treatments. Accordingly, the disclosure relates to the administration of cationic peptides or cationic peptide complexes in combination with another conventional anti-neoplastic agent, either concomitantly or sequentially, in order to enhance the therapeutic effect of the anti-neoplastic agent or overcome cellular resistance to such anti-neoplastic agent.

Pharmaceutical compounds that may be used for combinatory anti-tumor therapy include, but are not limited to: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

The dosage and selection of the therapeutic agent can be determined by a health care professional based on common knowledge in the art.

In some embodiments, the cationic peptide is complexed with a viral polynucleotide. In some embodiments, the viral polynucleotide is a viral DNA sequence that is relevant for its pathogenicity. In some embodiments, the viral DNA sequence comprises a CpG motif. CpG is the dinucleotide CG, linked by a phosphate group. CpG motifs are present in a variety of DNA viruses and CpG signaling is relevant to host defense against viral infections. Any CpG motif known in the art as being relevant for viral infection is suitable for use in the methods and compositions of the present invention. In some embodiments, the cationic peptide is complexed with a synthetic CpG-ODN. CpG ODNs are short synthetic single-stranded DNA molecules containing unmethylated CpG dinucleotides. CpG ODNs possess a partially or completely phophorothioated (PS) backbone. The synthetic CpG-ODN may be a CpG-A ODN, CpG-B ODN, or CpG-C ODN. CpG-A ODNs comprise a central phosphodiester (PO)-linked CpG-containing palindromic motif and a PS-modified 3′ or 5′ poly-G string. An exemplary sequence of a CpG-A ODN includes: 5′-GGGGGACGATCGTCGGGGGG-3′ (SEQ ID NO: 29). CpG-B ODNs contain a fully PS-modified backbone with one or more CpG dinucleotides. An exemplary sequence of a CpG-B ODN includes 5′-TCGTCGTTTTGTCGTTTTGTCGTT-3′ (SEQ ID NO: 30). CpG-C ODNs comprise a complete PS-modified backbone and a CpG-containing palindromic motif. An exemplary CpG-C ODN includes: 5′-TCGTCGTCGTTCGAACGACGTTGAT-3′ (SEQ ID NO: 31).

### F. Synthesis and Characteristics of Cationic Peptides

The present invention is directed generally to the use of cationic peptides to enhance an immune response, reduce viral inflammation, or treat or prevent a viral infection. The cationic peptides useful in the methods and compositions described herein may be produced by a variety of methods (e.g., chemical or recombinant). Suitable cationic peptides include, but are not limited to, naturally occurring cationic peptides, which have been isolated, and derivatives or analogs thereof. An “isolated peptide, polypeptide, or protein” is an amino acid sequence that is essentially free from contaminating cellular components, such as carbohydrate, lipid, nucleic acid (DNA or RNA), or other proteinaceous impurities associated with the polypeptide in nature. Preferably, the isolated polypeptide is sufficiently pure for therapeutic use at the desired dose.

The cationic peptide useful in the methods and compositions disclosed herein may be a recombinant peptide or a synthetic peptide, and is preferably a synthetic peptide. Peptides may be synthesized by standard chemical methods, including synthesis by automated procedure. Peptide analogues may be synthesized based on the standard solid-phase Fmoc protection strategy with HATU as the coupling agent. The peptide is cleaved from the solid-phase resin with trifluoroacetic acid containing appropriate scavengers, which also deprotects side chain functional groups. Peptide analogues may also be synthesized by liquid-phase synthesis. Crude peptide is further purified using preparative reversed-phase chromatography. Other purification methods, such as partition chromatography, gel filtration, gel electrophoresis, or ion-exchange chromatography may be used. Other synthesis techniques, known in the art, such as the tBoc protection strategy, or use of different coupling reagents or the like can be employed to produce equivalent peptides. Peptides may be synthesized as a linear molecule or as branched molecules. Branched peptides typically contain a core peptide that provides a number of attachment points for additional peptides. Lysine is most commonly used for the core peptide because it has one carboxyl functional group and two (alpha and epsilon) amine functional groups. Other diamino acids can also be used. Preferably, either two or three levels of geometrically branched lysines are used; these cores form a tetrameric and octameric core structure, respectively.

The cationic peptides useful in the methods and compositions disclosed herein are peptides that typically exhibit a positive charge at a pH ranging from about 3 to about 10 (i.e., has an isoelectric point of at least about 9), and contains at least one basic amino acid (e.g., arginine, lysine, histidine). In addition, the cationic peptide generally comprises an amino acid sequence having a molecular mass of about 0.5 kDa (i.e., approximately five amino acids in length) to about 10 kDa (i.e., approximately 100 amino acids in length), or a molecular mass of any integer, or fraction thereof (including a tenth and one hundredth of an integer), ranging from about 0.5 kDa to about 10 kDa. In some embodiments, the cationic peptide has a molecular mass ranging from about 0.5 kDa to about 5 kDa (i.e., approximately from about 5 amino acids to about 45 amino acids in length), from about 1 kDa to about 4 kDa (i.e., approximately from about 10 amino acids to about 35 amino acids in length), or from about 1 kDa to about 2 kDa (i.e., approximately from about 10 amino acids to about 18 amino acids in length). In another preferred embodiment, the cationic peptide is part of a larger peptide or polypeptide sequence having, for example, a total of up to 100 amino acids, up to 50 amino acids, up to 35 amino acids, or up to 15 amino acids. The methods of the invention contemplate a cationic peptide having an amino acid sequence of 5 to 100 amino acids, with the number of amino acids making up the peptide sequence comprising any integer in that range. The cationic peptide may exhibit antiviral activity, anti-inflammatory activity, immune enhancing activity, anticancer activity, and synergistic activity with other antiviral peptides or antiviral compounds, or a combination thereof. Exemplary peptides include, but are not limited to, cathelicidins, such as indolicidin and derivatives or analogues thereof from bovine neutrophils (Falla et al., J. Biol. Chem. 277:19298, 1996).

In certain embodiments, the cationic peptides are indolicidins or analogs or derivatives thereof. Natural indolicidins may be isolated from a variety of organisms, and, for example, the indolicidin isolated from bovine neutrophils is a 13 amino acid peptide, which is tryptophan-rich and amidated at the C-terminus (see Selsted et al., J. Biol. Chem. 267:4292, 1992). As noted above, in some embodiments, an indolicidin or analog or derivative thereof comprises 5 to 45 amino acids, 7 to 35 amino acids, 8 to 25 amino acids, or 10 to 14 amino acids. The indolicidins or analogs or derivatives thereof may be used at a concentration of about 5-25 μg/mL. In some embodiments, the indolicidin or analog or derivative thereof is delivered to the site of viral infection at a concentration of about 5-25 μg/mL. In certain embodiments, the cationic peptide is an indolicidin or an analog or derivative thereof in any one of the aforementioned compositions. In some embodiments, the antiviral cationic peptide used in the methods and compositions disclosed herein is a peptide of up to 35 amino acids, comprising one of the sequences in Table 1, infra.

TABLE 1
Exemplary Cationic Peptides

| Peptide | | |
|---|---|---|
| name | SEQ ID NO | Peptide sequence |
| LL-37 | SEQ ID NO: 1 | L L G D F F R K S K E K I G K E F K R I V Q R I |
| | | K D F L R N L V P R T E S |
| Omiganan | SEQ ID NO: 2 | I L R W P W W P W R R K |
| 11B32CN | SEQ ID NO: 3 | K R K W P W W P W R L I |
| 11B36CN | SEQ ID NO: 4 | I L K W V W W V W R R K |
| 11E3CN | SEQ ID NO: 5 | i L K K W P W W P W R R k |
| 11F4CN | SEQ ID NO: 6 | I L R W V W W V W R R K |
| 11F5CN | SEQ ID NO: 7 | I L R R W V W W V W R R K |
| 11F12CN | SEQ ID NO: 8 | R L W V W W V W R R K |
| 11F17CN | SEQ ID NO: 9 | R L W V W W V W R R |
| 11F50CN | SEQ ID NO: 10 | R L G G G W V W W V W R R |
| 11F56CN | SEQ ID NO: 11 | R L W W V V W W R R |
| 11F63CN | SEQ ID NO: 12 | R L V V W W V V R R |
| 11F64CN | SEQ ID NO: 13 | R L F V W W V F R R |
| 11F66CN | SEQ ID NO: 14 | R L V V W V V W R R |
| 11F67CN | SEQ ID NO: 15 | r L W V W W V W R R |
| 11F68CN | SEQ ID NO: 16 | R L W V W W V W R r |
| 11F93CN | SEQ ID NO: 17 | W V R L W W R R V W |
| 11G27CN | SEQ ID NO: 18 | W P W W P W R R K |
| 11J02CN | SEQ ID NO: 19 | W R W W K P K W R W P K W |
| 11J02ACN | SEQ ID NO: 20 | W R W W K P K W R W P K W |
| 11J30CN | SEQ ID NO: 21 | W R W W K V A W R W V K W |
| 11J36CN | SEQ ID NO: 22 | W R W W K V W R W V K W |
| 11J58CN | SEQ ID NO: 23 | W (Orn) W W (Orn) V A W (Orn) W V (Orn) W |
| 11J67CN | SEQ ID NO: 24 | W (Orn) W W (Orn) P (Orn) W (Orn) W P (Orn) W |
| 11J68CN | SEQ ID NO: 25 | W (Dab) W W (Dab) P (Dab) W (Dab) W P (Dab) W |
| Nt- | SEQ ID NO: 26 | Nt-acryloyl I L R W P W W P W R R K CN |
| acryloyl- | | |
| 11B7CN | | |
| Nt- | SEQ ID NO: 27 | Nt-glucosyl W R W W K V W R W V K W CN |
| glucosyl- | | |
| 11J36CN | | |
| Nt- | SEQ ID NO: 28 | Nt-glucosyl W R W W K V V W R W V K W CN |
| glucosyl- | | |
| 11J38CN | | |

Nt prefix = N-terminal modification
CN suffix = amidated C-terminus
H suffix = homoserine at C-terminus
R suffix = retro-synthesized peptide
Orn = ornithine
Dab = diamino butyric acid
Upper case letter = L-enantiomer amino acid
Lower case letter = D-enantiomer amino acid

Thus in some embodiments, the cationic peptide is LL-37. In some embodiments, the cationic peptide is LL-22, a 22 amino acid variant of LL-37. In some embodiments, the cationic peptide is an N-terminal 22 amino acid truncation of LL-37. In some embodiments, the cationic peptide is a C-terminal 22 amino acid truncation of LL-37. In some embodiments, the cationic peptide is a fragment of LL-37. In some embodiments, the fragment of LL-37 has residues 1-22 of SEQ ID NO: 1. In some embodiments, the fragment of LL-37 has residues 2-23 of SEQ ID NO: 1. In some embodiemnts, the fragment of LL-37 has residues 3-24 of SEQ ID NO: 1. In some embodiemnts, the fragment of LL-37 has residues 4-25 of SEQ ID NO: 1. In some embodiments, the fragment of LL-37 has residues 5-26 of SEQ ID NO: 1. In some embodiments, the fragment of LL-37 has residues 6-27 of SEQ ID NO: 1. In some embodiments, the fragment of LL-37 has residues 7-28 of SEQ ID NO: 1. In some embodiments, the fragment of LL-37 has residues 8-29 of SEQ ID NO: 1. In some embodiments, the fragment of LL-37 has residues 9-30 of SEQ ID NO: 1. In some embodiments, the fragment of LL-37 has residues 10-31 of SEQ ID NO: 1. In some embodiments, the fragment of LL-37 has residues 11-32 of SEQ ID NO: 1. In some embodiments, the fragment of LL-37 has residues 12-33 of SEQ ID NO: 1. In some embodiments, the fragment of LL-37 has residues 13-34 of SEQ ID NO: 1. In some embodiemnts, the fragment of LL-37 has residues 14-35 of SEQ ID NO: 1. In some embodiemnts, the fragment of LL-37 has residues 15-36 of SEQ ID NO: 1. In some embodiemnts, the fragment of LL-37 has residues 16-37 of SEQ ID NO: 1. LL-37 variants are described in van der Does et al. J Immunol 2010 185:1442-1449. In other embodiments, the cationic peptide is omiganan. In some embodiments, the cationic peptide is 11B32CN. In other embodiments, the cationic peptide is 11B36CN. In some embodiments, the cationic peptide is 11E3CN. In other embodiments, the cationic peptide is 11F4CN. In some embodiments, the cationic peptide is 11F5CN. In other embodiments, the cationic peptide is 11F12CN. In some embodiments, the cationic peptide is 11F17CN. In other embodiments, the cationic peptide is 11F50CN. In some embodiments, the cationic peptide is 11F56CN. In other embodiments, the cationic peptide is 11F63CN. In some embodiments, the cationic peptide is 11F64CN. In other embodiments, the cationic peptide is 11F66CN. In some embodiments, the cationic peptide is 11B32CN. In other embodiments, the cationic peptide is 11F67CN. In some embodiments, the cationic peptide is 11F68CN. In other embodiments, the cationic peptide is 11F93CN. In some embodiments, the cationic peptide is 11G27CN. In other embodiments, the cationic peptide is 11J02CN. In some embodiments, the cationic peptide is 11J02ACN. In other embodiments, the cationic peptide is 11J30CN. In some embodiments, the cationic peptide is 11J36CN. In other embodiments, the cationic peptide is 11J58CN. In some embodiments, the cationic peptide is 11J67CN. In other embodiments, the cationic peptide is 11J68CN. In some embodiments, the cationic peptide is Nt-acryloyl-11B7CN. In other embodiments, the cationic peptide is Nt-glucosyl-11J36CN. In some embodiments, the cationic peptide is Nt-glucosyl-11J38CN.

In some embodiments, the cationic peptide is provided in conjunction with a counter anion. The counter anion may be any pharmaceutically acceptable counter anion. In some embodiments, the cationic peptide is omiganan pentahydrochloride.

The cationic peptide of the present invention may be an analog or derivative thereof. As used herein, the terms “derivative” and “analog” when referring to a cationic peptide, polypeptide, or fusion protein, refer to any cationic peptide, polypeptide, or fusion protein that retain essentially the same (at least 50%, 60% and preferably greater than 70, 80, or 90%) or enhanced biological function or activity as such cationic peptide, as noted above. The biological function or activity of such analogs and derivatives can be determined using standard methods (e.g., antiviral, anti-inflammatory, DNA and/or protein synthesis inhibitor), such as with the assays described herein. For example, an analog or derivative may be a proprotein that can be activated by cleavage to produce an active antiviral cationic peptide. Alternatively, a cationic peptide analog or derivative thereof can be identified by the ability to specifically bind anti-cationic peptide antibodies.

The cationic peptide analog or derivative may have, for example, one or more deletion, insertion, or modification of any amino acid residue, including the N- or C-terminal amino acids. Within the scope of this invention are modified antiviral cationic peptides, such as, for example, peptides having an acetylated, acylated, acryloylated, alkylated, glycosylated (e.g., glucosylated), PEGylated, myristylated, and the like N-terminal amino acid modification; having an esterified, amidated, homoserinelhomoserine lactone, or caprolactam C-terminal amino acid modification; or having a polyalkylene glycol (e.g., polyethylene glycol) conjugated to any free amino group. A preferred modification of the C-terminal amino acid is amidation. An analog or derivative may also be an antiviral cationic peptide fusion protein. Fusion proteins, or chimeras, include fusions of one or more antiviral cationic peptides, and fusions of cationic peptides with non-cationic peptides. Additionally, the peptide may be modified to form a polymer-modified peptide. The peptides may also be labeled, such as with a radioactive label, a fluorescent label, a mass spectrometry tag, biotin, and the like.

Another example of an analog or derivative includes a cationic peptide that has one or more conservative amino acid substitutions, as compared with the amino acid sequence of a cationic peptide of the present invention. Among the common amino acids, a “conservative amino acid substitution” is illustrated, for example, by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine, or a combination thereof. Furthermore, an analog or derivative of a cationic peptide may include, for example, non-protein amino acids, such as precursors of normal amino acids (e.g., homoserine and diaminopimelate), intermediates in catabolic pathways (e.g., pipecolic acid and D-enantiomers of normal amino acids), and amino acid analogs (e.g., azetidine-2-carboxylic acid and canavanine).

The indolicidins or analogs or derivatives thereof of the present invention may be used individually, or may be used in combination with one or more different indolicidins or analogs or derivatives thereof, with one or more cationic peptides, or with one or more conventional antiviral agents, as described herein. Thus, combinations (including synergistic combinations) of a cationic peptide and an additional antiviral agent may permit a reduction in the dosage of one or both agents in order to achieve a similar or improved therapeutic effect. This would allow the use of smaller doses and, therefore, would decrease the potential incidence of toxicity and lower costs of expensive antivirals. Concurrent or sequential administration of a cationic peptide formulation and a conventional antiviral agent composition is expected to provide more effective treatment of infections caused by a virus. In particular, successful treatment or prevention of viral infections can be achieved by using the cationic peptides and conventional antiviral agents at doses below what is normally a therapeutically effective dose when these antivirals are used individually. Alternatively, the conventional antiviral agent and cationic peptide can be administered using a normally effective therapeutic dose for each antiviral, but wherein the combination of the two agents provides even more potent effects.

As noted above, the cationic peptide may be used in a combination with other known antiviral agents. In some embodiments, the combination may have a synergistic effect. In other embodiments, the combination may have an additive effect. Exemplary antiviral agents include, but are not limited to, abacavir, acyclovir, adefovir, amantadine hydrochloride, amprenavir, atazanavir, bocepravir, cidofovir, cobicistatlamivudine, daclatasvir, darunavir, dasabuvir, delavirdine, didanosine, didanosinezalcitabine, dolutegravir, edoxudine, efavirenz, elvitegravir, emtricitabine, elvitegravir enfuvirtide, entecavir, etravirine, famciclovir, fomivirsen foscarnet, fosamprenavir, ganciclovir sodium, idoxuridine, indinavir, interferon alpha, ledipasvir, lopinavir, maraviroc, nelfinavir, nevirapine, ombitasvir, oseltamivir, paritaprevir, peginterferon alpha-2a, peginterferon alpha-2b, peramivir, raltegravir, ribavirin, rilpivirine, rimantadine, ritonavir, ribavirin, saquinavir, simeprevir, sofosbovir, sorivudine, stavudine, telaprevir, telbivudine, tenofovir, tipranavir, trifluridine, valacyclovir, valganciclovir, vidarabin, zanamivir and zidovudine.

In some embodiments, the composition disclosed in the methods disclosed herein further comprises an additional immune modulator. For example, the additional immune modulator may be imiquimod, polyI:C, CpG DNA, or any other viral RNA or viral DNA.

As noted above, the present invention contemplates analogs or derivatives of natural cationic peptides, which analogs or derivatives may be recombinantly produced by the presently described methods. Nucleotide sequences encoding conservative amino acid analogs or derivatives can be obtained, for example, by oligonucleotide-directed mutagenesis, linker-scanning mutagenesis, mutagenesis using the polymerase chain reaction, and the like (see Ausubel, 1995, at page 8-10 through page 8-22; and McPherson (ed.), Directed Mutagenesis: A Practical Approach, IRL Press, 1991).

Although one objective in constructing a cationic peptide variant may be to improve its activity, it may also be desirable to alter the amino acid sequence of a naturally occurring cationic peptide to enhance its production in a recombinant host cell. The presence of a particular codon may have an adverse effect on expression in a particular host; therefore, a DNA sequence encoding the desired cationic peptide is optimized for a particular host system, such as prokaryotic or eukaryotic cells. For example, a nucleotide sequence encoding a radish cationic peptide may include a codon that is commonly found in radish, but is rare for E. coli. The presence of a rare codon may have an adverse effect on protein levels when the radish cationic peptide is expressed in recombinant E. coli. Methods for altering nucleotide sequences to alleviate the codon usage problem are well known to those of skill in the art (see, e.g., Kane, Curr. Opin. Biotechnol. 6:494, 1995; Makrides, Microbial. Rev. 60:512, 1996; and Brown (Ed.), Molecular Biology LabFax, BIOS Scientific Publishers, Ltd., 1991, which provides a Codon Usage Table at page 245 through page 253).

Peptides may be synthesized by recombinant techniques (see e.g., U.S. Pat. No. 5,593,866) and a variety of host systems are suitable for production of the cationic peptides and analogues or derivatives thereof, including bacteria (e.g., E. coli), yeast (e.g., Saccharomyces cerevisiae), insect (e.g., Sf9), and mammalian cells (e.g., CHO, COS-7). Many expression vectors have been developed and are available for each of these hosts. Generally, vectors that are functional in bacteria are used in this invention. However, at times, it may be preferable to have vectors that are functional in other hosts. Vectors and procedures for cloning and expression in E. coli are discussed herein and, for example, in Sambrook et al. (Molecular Cloning: A Laboratory Manual, Cold Spring Harbor Laboratory Press, Cold Spring Harbor, N.Y., 1987) and in Ausubel et al., 1995.

A DNA sequence encoding a cationic peptide is introduced into an expression vector appropriate for the host. In preferred embodiments, the gene is cloned into a vector to create a fusion protein. The fusion partner is chosen to contain an anionic region, such that a bacterial host is protected from the toxic effect of the peptide. This protective region effectively neutralizes the antimicrobial effects of the peptide and also may prevent peptide degradation by host proteases. The fusion partner (carrier protein) of the invention may further function to transport the fusion peptide to inclusion bodies, the periplasm, the outer membrane, or the extracellular environment. Carrier proteins suitable in the context of this invention specifically include, but are not limited to, glutathione-S-transferase (GST), protein A from Staphylococcus aureus, two synthetic IgG-binding domains (ZZ) of protein A, outer membrane protein F, β-galactosidase (lacZ), and various products of bacteriophage λ and bacteriophage T7. From the teachings provided herein, it is apparent that other proteins may be used as carriers. Furthermore, the entire carrier protein need not be used, as long as the protective anionic region is present. To facilitate isolation of the peptide sequence, amino acids susceptible to chemical cleavage (e.g., CNBr) or enzymatic cleavage (e.g., VB protease, trypsin) are used to bridge the peptide and fusion partner. For expression in E. coli, the fusion partner is preferably a normal intracellular protein that directs expression toward inclusion body formation. In such a case, following cleavage to release the final product, there is no requirement for renaturation of the peptide. In the present invention, the DNA cassette, comprising fusion partner and peptide gene, may be inserted into an expression vector, which can be a plasmid, virus or other vehicle known in the art. Preferably, the expression vector is a plasmid that contains an inducible or constitutive promoter to facilitate the efficient transcription of the inserted DNA sequence in the host. Transformation of the host cell with the recombinant DNA may be carried out by Ca⁺⁺-mediated techniques, by electroporation, or other methods well known to those skilled in the art. Briefly, a DNA fragment encoding a peptide is derived from an existing cDNA or genomic clone or synthesized. A convenient method is amplification of the gene from a single-stranded template. The template is generally the product of an automated oligonucleotide synthesis. Amplification primers are derived from the 5′ and 3′ ends of the template and typically incorporate restriction sites chosen with regard to the cloning site of the vector. If necessary, translational initiation and termination codons can be engineered into the primer sequences. The sequence encoding the protein may be codon optimized for expression in the particular host. Thus, for example, if the analogue fusion protein is expressed in bacteria, codons are optimized for bacterial usage. Codon optimization is accomplished by automated synthesis of the entire gene or gene region, ligation of multiple oligonucleotides, mutagenesis of the native sequence, or other techniques known to those in the art.

In some embodiments, the vector is capable of replication in bacterial cells. Thus, the vector may contain a bacterial origin of replication. Preferred bacterial origins of replication include f1-ori and col E1 ori, especially the ori derived from pUC plasmids. Low copy number vectors (e.g., pPD100) may also be used, especially when the product is deleterious to the host. The plasmids also preferably include at least one selectable marker that is functional in the host. A selectable marker gene confers a phenotype on the host that allows transformed cells to be identified and/or selectively grown. Suitable selectable marker genes for bacterial hosts include the chloramphenicol resistance gene (Cm^{r}), ampicillin resistance gene (Amp^{r}), tetracycline resistance gene (Tc^{r}) kanamycin resistance gene (Kan^{r}), and others known in the art. To function in selection, some markers may require a complementary deficiency in the host. The vector may also contain a gene coding for a repressor protein, which is capable of repressing the transcription of a promoter that contains a repressor binding site. Altering the physiological conditions of the cell can depress the promoter. For example, a molecule may be added that competitively binds the repressor, or the temperature of the growth media may be altered. Repressor proteins include, but are not limited to the E. coli lac1 repressor (responsive to induction by IPTG), the temperature sensitive λcl857 repressor, and the like.

At minimum, the expression vector should contain a promoter sequence. However, other regulatory sequences may also be included. Such sequences include an enhancer, ribosome binding site, transcription termination signal sequence, secretion signal sequence, origin of replication, selectable marker, and the like. The regulatory sequences are operably linked with one another to allow transcription and subsequent translation. In preferred aspects, the plasmids used herein for expression include a promoter designed for expression of the proteins in bacteria. Suitable promoters, including both constitutive and inducible promoters, are widely available and are well known in the art. Commonly used promoters for expression in bacteria include promoters from T7, T3, T5, and SP6 phages, and the trp, 1pp, and lac operons. Hybrid promoters (see, U.S. Pat. No. 4,551,433), such as tac and trc, may also be used. Examples of plasmids for expression in bacteria include the pET expression vectors pET3a, pET 11a, pET 12a-c, and pET 15b (see U.S. Pat. No. 4,952,496; available from Novagen, Madison, Wis.). Low copy number vectors (e.g., pPD1 00) can be used for efficient overproduction of peptides deleterious to the E. coli host (Dersch et al., FEMS Microbial. Lett. 123: 19, 1994). Bacterial hosts for the T7 expression vectors may contain chromosomal copies of DNA encoding T7 RNA polymerase operably linked to an inducible promoter (e.g., lacUV promoter; see, U.S. Pat. No. 4,952,496), such as found in the E. coli strains HMS174(DE3)plysS, BL21(DE3)plysS, HMS174(DE3) and BL21 (DE3). T7 RNA polymerase can also be present on plasmids compatible with the T7 expression vector. The polymerase may be under control of a lambda promoter and repressor (e.g., pGP1-2; Tabor and Richardson, Proc. Natl. Acad. Sci. USA 82: 1074, 1985).

In some aspects, the sequence of nucleotides encoding the peptide also encodes a secretion signal, such that the resulting peptide is synthesized as a precursor protein (i.e., proprotein), which is subsequently processed and secreted. The resulting secreted peptide or fusion protein may be recovered from the periplasmic space or the fermentation medium. Sequences of secretion signals suitable for use are widely available and are well known (von Heijne, J. Mol. Biol. 184:99-105, 1985).

The peptide product is isolated by standard techniques, such as affinity, size exclusion, or ionic exchange chromatography, HPLC and the like. An isolated peptide should preferably show a major band by Coomassie blue stain of SDS-PAGE, which is preferably at least 75%, 80%, 90%, or 95% of the purified peptide, polypeptide, or fusion protein.

### G. Route of Administration

A therapeutically effective amount of the cationic peptides of the present invention can be administered according to any route of administration, without limitation, known in the art (e.g., topical, enteral, parenteral, oral, sublingual, nasal, inhalation, intranasal, injection, bladder wash-out, vagina, rectal, suppository etc). It is within the skill in the art to determine the appropriate route of administration for a given subject. For example, the cationic peptide may be administered topically or parenterally. In some embodiments, the administration is epicutaneous, inhalation, intranasal, an enema, eye drops, ear drops, or through a mucous membrane. In some embodiments, the formulations of the present invention are, for example, particularly suitable for topical (e.g., creams, ointments, skin patches, eye drops, ear drops, shampoos) application or administration. In some embodiments, the cationic peptide is administered with an additional antiviral agent. In some embodiments, the parenteral administration is subcutaneous, intravenous, intramuscular, intraarterial, intraabdominal, intraperitoneal, intraarticular, intraocular or retrobulbar, intraaural, intrathecal, intracavitary, intracelial, intraspinal, intrapulmonary or transpulmonary, intrasynovial, or intraurethral injection or infusion. The pharmaceutical compositions of the present invention are formulated so as to allow the antiviral cationic peptide contained therein to be bioavailable upon administration of the composition to a subject. The level of peptide in serum and other tissues after administration can be monitored by various well-established techniques, such as chromatographic or antibody based (e.g., ELISA) assays. In some embodiments, the antiviral cationic peptides and analogs and derivatives thereof, as described herein, are formulated for topical application to a target site on a subject in need thereof.

The compositions may be administered to a subject as a single dosage unit (e.g., a tablet, capsule, or gel). Alternatively, the compositions may be administered as a plurality of dosage units (e.g., in aerosol form). For example, the antiviral cationic peptide formulations may be sterilized and packaged in single-use, plastic laminated pouches or plastic tubes of dimensions selected to provide for routine, measured dispensing. In one example, the container may have dimensions anticipated to dispense 0.5 ml of the antiviral cationic peptide composition (e.g., a gel form) to a limited area of the target surface on or in a subject to treat or prevent an infection. A typical target, for example, is in the immediate vicinity of the insertion site of an intravenous catheter, where the target surface usually has an area of about two square centimeters.

### H. Cationic Peptide Formulations and Concentrations

As noted above, the present invention provides methods for enhancing an immune response, reducing inflammation, treating or preventing viral infections by administering to a patient a therapeutically effective amount of an cationic peptide, preferably an indolicidin or analog or derivative thereof, as described herein. The cationic peptide is preferably part of a pharmaceutical composition when used in the methods of the present invention. The pharmaceutical composition will include at least one of a pharmaceutically acceptable vehicle, carrier, diluent, or excipient, in addition to one or more cationic peptide and, optionally, other components. Pharmaceutically acceptable excipients for therapeutic use are well known in the pharmaceutical art, and are described herein and, for example, in Remington's Pharmaceutical Sciences, Mack Publishing Co. (A. R. Gennaro, ed., 18^{th} Edition, 1990) and in CRC Handbook of Food, Agent, and Cosmetic Excipients, CRC Press LLC (S.C. Smolinski, ed., 1992).

The cationic peptide composition may be provided in various forms, depending on the amount and number of different pharmaceutically acceptable excipients present. For example, the cationic peptide composition may be in the form of a solid, a semi-solid, a liquid, a lotion, a cream, an ointment, a cement, a paste, a gel, or an aerosol. In some embodiments, the cationic peptide formulation is in the form of a gel. The pharmaceutically acceptable excipients suitable for use in the cationic peptide formulation compositions as described herein may include, for example, a viscosity-increasing agent, a buffering agent, a solvent, a humectant, a preservative, a chelating agent, an oleaginous compound, an emollient, an antioxidant, an adjuvant, and the like. The function of each of these excipients is not mutually exclusive within the context of the present invention. For example, glycerin may be used as a solvent or as a humectant or as a viscosity-increasing agent. In one preferred embodiment, the formulation is a composition comprising an antiviral cationic peptide, a viscosity-increasing agent, and a solvent, which is for example, at a target site for implanted or indwelling medical devices, as described herein.

In some embodiments, the cationic peptide is provided in conjunction with a counter anion. The counter anion may be any pharmaceutically acceptable counter anion. The counter anion may include anionic groups such as carboxylates, phosphonates, sulphates and sulphonates. The cationic peptide may be provided in the form of any pharmaceutically acceptable salt such as but not limited to trifluoroacetate, acetate, chloride and sulfate. In some embodiments, the cationic peptide is omiganan pentahydrochloride.

In some embodiments, the cationic peptide is delivered to the site of viral infection at a concentration of 1-25 μg/mL. In some embodiments, the cationic peptide is delivered to the site of viral infection at a concentration of 1-5 μg/mL. In some embodiments, the cationic peptide is delivered to the site of viral infection at a concentration of 1-10 μg/mL. In some embodiments, the cationic peptide is delivered to the site of viral infection at a concentration of 1-15 μg/mL. In some embodiments, the cationic peptide is delivered to the site of viral infection at a concentration of 1-20 μg/mL. In some embodiments, the cationic peptide is delivered to the site of viral infection at a concentration of 5-25 μg/mL. In some embodiments, the cationic peptide is delivered to the site of viral infection at a concentration of 5-10 μg/mL. In some embodiments, the cationic peptide is delivered to the site of viral infection at a concentration of 5-15 μg/mL. In some embodiments, the cationic peptide is delivered to the site of viral infection at a concentration of 5-20 μg/mL. In some embodiments, the cationic peptide is delivered to the site of viral infection at a concentration of 10-20 μg/mL. In some embodiments, the cationic peptide is delivered to the site of viral infection at a concentration of 10-25 μg/mL. In some embodiments, the cationic peptide is delivered to the site of viral infection at a concentration of 15-25 μg/mL. In some embodiments, the cationic peptide is delivered to the site of viral infection at a concentration of 1 μg/mL. In some embodiments, the cationic peptide is delivered to the site of viral infection at a concentration of 2 μg/mL. In some embodiments, the cationic peptide is delivered to the site of viral infection at a concentration of 3 μg/mL. In some embodiments, the cationic peptide is delivered to the site of viral infection at a concentration of 4 μg/mL. In some embodiments, the cationic peptide is delivered to the site of viral infection at a concentration of 5 μg/mL. In some embodiments, the cationic peptide is delivered to the site of viral infection at a concentration of 6 μg/mL. In some embodiments, the cationic peptide is delivered to the site of viral infection at a concentration of 7 μg/mL. In some embodiments, the cationic peptide is delivered to the site of viral infection at a concentration of 8 μg/mL. In some embodiments, the cationic peptide is delivered to the site of viral infection at a concentration of μg/mL. In some embodiments, the cationic peptide is delivered to the site of viral infection at a concentration of 10 μg/mL. In some embodiments, the cationic peptide is delivered to the site of viral infection at a concentration of 11 μg/mL. In some embodiments, the cationic peptide is delivered to the site of viral infection at a concentration of 12 μg/mL. In some embodiments, the cationic peptide is delivered to the site of viral infection at a concentration of 13 μg/mL. In some embodiments, the cationic peptide is delivered to the site of viral infection at a concentration of 14 μg/mL. In some embodiments, the cationic peptide is delivered to the site of viral infection at a concentration of 15 μg/mL. In some embodiments, the cationic peptide is delivered to the site of viral infection at a concentration of 16 μg/mL. In some embodiments, the cationic peptide is delivered to the site of viral infection at a concentration of 17 μg/mL. In some embodiments, the cationic peptide is delivered to the site of viral infection at a concentration of 18 μg/mL. In some embodiments, the cationic peptide is delivered to the site of viral infection at a concentration of 19 μg/mL. In some embodiments, the cationic peptide is delivered to the site of viral infection at a concentration of 20 μg/mL. In some embodiments, the cationic peptide is delivered to the site of viral infection at a concentration of 21 μg/mL. In some embodiments, the cationic peptide is delivered to the site of viral infection at a concentration of 22 μg/mL. In some embodiments, the cationic peptide is delivered to the site of viral infection at a concentration of 23 μg/mL. In some embodiments, the cationic peptide is delivered to the site of viral infection at a concentration of 24 μg/mL. In some embodiments, the cationic peptide is delivered to the site of viral infection at a concentration of 25 μg/mL.

In some embodiments, the cationic peptide is delivered to the site of viral infection at a concentration of about 1-25 μg/mL. In some embodiments, the cationic peptide is delivered to the site of viral infection at a concentration of about 1-5 μg/mL. In some embodiments, the cationic peptide is delivered to the site of viral infection at a concentration of about 1-10μg/mL. In some embodiments, the cationic peptide is delivered to the site of viral infection at a concentration of about 1-15 μg/mL. In some embodiments, the cationic peptide is delivered to the site of viral infection at a concentration of about 1-20 μg/mL. In some embodiments, the cationic peptide is delivered to the site of viral infection at a concentration of about 5-25 μg/mL. In some embodiments, the cationic peptide is delivered to the site of viral infection at a concentration of about 5-10 μg/mL. In some embodiments, the cationic peptide is delivered to the site of viral infection at a concentration of about 5-15 μg/mL. In some embodiments, the cationic peptide is delivered to the site of viral infection at a concentration of about 5-20 μg/mL. In some embodiments, the cationic peptide is delivered to the site of viral infection at a concentration of about 10-20 μg/mL. In some embodiments, the cationic peptide is delivered to the site of viral infection at a concentration of about 10-25 μg/mL. In some embodiments, the cationic peptide is delivered to the site of viral infection at a concentration of about 15-25 μg/mL. In some embodiments, the cationic peptide is delivered to the site of viral infection at a concentration of about 1 μg/mL. In some embodiments, the cationic peptide is delivered to the site of viral infection at a concentration of about 2 μg/mL. In some embodiments, the cationic peptide is delivered to the site of viral infection at a concentration of about 3 μg/mL. In some embodiments, the cationic peptide is delivered to the site of viral infection at a concentration of about 4 μg/mL. In some embodiments, the cationic peptide is delivered to the site of viral infection at a concentration of about 5 μg/mL. In some embodiments, the cationic peptide is delivered to the site of viral infection at a concentration of about 6 μg/mL. In some embodiments, the cationic peptide is delivered to the site of viral infection at a concentration of about 7 μg/mL. In some embodiments, the cationic peptide is delivered to the site of viral infection at a concentration of about 8 μg/mL. In some embodiments, the cationic peptide is delivered to the site of viral infection at a concentration of about 9 μg/mL. In some embodiments, the cationic peptide is delivered to the site of viral infection at a concentration of about 10 μg/mL. In some embodiments, the cationic peptide is delivered to the site of viral infection at a concentration of about 11 μg/mL. In some embodiments, the cationic peptide is delivered to the site of viral infection at a concentration of about 12 μg/mL. In some embodiments, the cationic peptide is delivered to the site of viral infection at a concentration of about 13 μg/mL. In some embodiments, the cationic peptide is delivered to the site of viral infection at a concentration of about 14 μg/mL. In some embodiments, the cationic peptide is delivered to the site of viral infection at a concentration of about 15 μg/mL. In some embodiments, the cationic peptide is delivered to the site of viral infection at a concentration of about 16 μg/mL. In some embodiments, the cationic peptide is delivered to the site of viral infection at a concentration of about 17 μg/mL. In some embodiments, the cationic peptide is delivered to the site of viral infection at a concentration of about 18 μg/mL. In some embodiments, the cationic peptide is delivered to the site of viral infection at a concentration of about 19 μg/mL. In some embodiments, the cationic peptide is delivered to the site of viral infection at a concentration of about 20 μg/mL. In some embodiments, the cationic peptide is delivered to the site of viral infection at a concentration of about 21 μg/mL. In some embodiments, the cationic peptide is delivered to the site of viral infection at a concentration of about 22 μg/mL. In some embodiments, the cationic peptide is delivered to the site of viral infection at a concentration of about 23 μg/mL. In some embodiments, the cationic peptide is delivered to the site of viral infection at a concentration of about 24 μg/mL. In some embodiments, the cationic peptide is delivered to the site of viral infection at a concentration of about 25 μg/mL.

In some embodiments, the cationic peptide is at a concentration of 0.01% to 10% (w/w). In some embodiments, the cationic peptide is at a concentration of 0.01% to 0.1% (w/w). In some embodiments, the cationic peptide is at a concentration of 0.01% to 0.5% (w/w). In some embodiments, the cationic peptide is at a concentration of 0.01% to 1% (w/w). In some embodiments, the cationic peptide is at a concentration of 0.01% to 2% (w/w). In some embodiments, the cationic peptide is at a concentration of 0.01% to 3% (w/w). In some embodiments, the cationic peptide is at a concentration of 0.01% to 4% (w/w). In some embodiments, the cationic peptide is at a concentration of 0.01% to 5% (w/w). In some embodiments, the cationic peptide is at a concentration of 0.01% to 6% (w/w). In some embodiments, the cationic peptide is at a concentration of 0.01% to 7% (w/w). In some embodiments, the cationic peptide is at a concentration of 0.01% to 8% (w/w). In some embodiments, the cationic peptide is at a concentration of 0.01% to 9% (w/w). In some embodiments, the cationic peptide is at a concentration of 0.05% to 10% (w/w). In some embodiments, the cationic peptide is at a concentration of 0.1% to 10% (w/w). In some embodiments, the cationic peptide is at a concentration of 0.5% to 10% (w/w). In some embodiments, the cationic peptide is at a concentration of 1% to 10% (w/w).

In some embodiments, the cationic peptide is at a concentration of 1.5% to 10% (w/w). In some embodiments, the cationic peptide is at a concentration of 2% to 10% (w/w). In some embodiments, the cationic peptide is at a concentration of 2.5% to 10% (w/w). In some embodiments, the cationic peptide is at a concentration of 1% to 2% (w/w). In some embodiments, the cationic peptide is at a concentration of 1% to 3% (w/w). In some embodiments, the cationic peptide is at a concentration of 1% to 4% (w/w). In some embodiments, the cationic peptide is at a concentration of 1% to 5% (w/w). In some embodiments, the cationic peptide is at a concentration of 1% to 6% (w/w). In some embodiments, the cationic peptide is at a concentration of 1% to 7% (w/w). In some embodiments, the cationic peptide is at a concentration of 1% to 8% (w/w). In some embodiments, the cationic peptide is at a concentration of 1% to 9% (w/w). In some embodiments, the cationic peptide is at a concentration of 1.5% to 2% (w/w). In some embodiments, the cationic peptide is at a concentration of 1.5% to 2.5% (w/w). In some embodiments, the cationic peptide is at a concentration of 1.5% to 3% (w/w). In some embodiments, the cationic peptide is at a concentration of 1.5% to 3.5% (w/w). In some embodiments, the cationic peptide is at a concentration of 0.5% to 1.5% (w/w). In some embodiments, the cationic peptide is at a concentration of 0.5% to 2% (w/w). In some embodiments, the cationic peptide is at a concentration of 0.5% to 2.5% (w/w). In some embodiments, the cationic peptide is at a concentration of 0.5% to 5% (w/w). In some embodiments, the cationic peptide is at a concentration of 0.01% (w/w). In some embodiments, the cationic peptide is at a concentration of 0.02% (w/w). In some embodiments, the cationic peptide is at a concentration of 0.03% (w/w). In some embodiments, the cationic peptide is at a concentration of 0.04% (w/w). In some embodiments, the cationic peptide is at a concentration of 0.05% (w/w). In some embodiments, the cationic peptide is at a concentration of 0.06% (w/w). In some embodiments, the cationic peptide is at a concentration of 0.07% (w/w). In some embodiments, the cationic peptide is at a concentration of 0.08% (w/w). In some embodiments, the cationic peptide is at a concentration of 0.09% (w/w). In some embodiments, the cationic peptide is at a concentration of 0.1% (w/w). In some embodiments, the cationic peptide is at a concentration of 0.2% (w/w). In some embodiments, the cationic peptide is at a concentration of 0.3% (w/w). In some embodiments, the cationic peptide is at a concentration of 0.4% (w/w). In some embodiments, the cationic peptide is at a concentration of 0.5% (w/w). In some embodiments, the cationic peptide is at a concentration of 0.6% (w/w). In some embodiments, the cationic peptide is at a concentration of 0.7% (w/w). In some embodiments, the cationic peptide is at a concentration of 0.8% (w/w). In some embodiments, the cationic peptide is at a concentration of 0.9% (w/w). In some embodiments, the cationic peptide is at a concentration of 1% (w/w). In some embodiments, the cationic peptide is at a concentration of 1.5% (w/w). In some embodiments, the cationic peptide is at a concentration of 2% (w/w). In some embodiments, the cationic peptide is at a concentration of 2.3% (w/w). In some embodiments, the cationic peptide is at a concentration of 2.5% (w/w). In some embodiments, the cationic peptide is at a concentration of 3% (w/w). In some embodiments, the cationic peptide is at a concentration of 3.5% (w/w). In some embodiments, the cationic peptide is at a concentration of 4.0% (w/w). In some embodiments, the cationic peptide is at a concentration of 4.5% (w/w). In some embodiments, the cationic peptide is at a concentration of 5% (w/w). In some embodiments, the cationic peptide is at a concentration of 5.5% (w/w). In some embodiments, the cationic peptide is at a concentration of 6% (w/w). In some embodiments, the cationic peptide is at a concentration of 6.5% (w/w). In some embodiments, the cationic peptide is at a concentration of 7% (w/w). In some embodiments, the cationic peptide is at a concentration of 7.5% (w/w). In some embodiments, the cationic peptide is at a concentration of 8% (w/w). In some embodiments, the cationic peptide is at a concentration of 8.5% (w/w). In some embodiments, the cationic peptide is at a concentration of 9% (w/w). In some embodiments, the cationic peptide is at a concentration of 9.5% (w/w). In some embodiments, the cationic peptide is at a concentration of 10% (w/w).

In some embodiments, the cationic peptide is at a concentration of about 0.01% to about 10% (w/w). In some embodiments, the cationic peptide is at a concentration of about 0.01% to about 0.1% (w/w). In some embodiments, the cationic peptide is at a concentration of about 0.01% to about 0.5% (w/w). In some embodiments, the cationic peptide is at a concentration of about 0.01% to about 1% (w/w). In some embodiments, the cationic peptide is at a concentration of about 0.01% to about 2% (w/w). In some embodiments, the cationic peptide is at a concentration of about 0.01% to about 3% (w/w). In some embodiments, the cationic peptide is at a concentration of about 0.01% to about 4% (w/w). In some embodiments, the cationic peptide is at a concentration of about 0.01% to about 5% (w/w). In some embodiments, the cationic peptide is at a concentration of about 0.01% to about 6% (w/w). In some embodiments, the cationic peptide is at a concentration of about 0.01% to about 7% (w/w). In some embodiments, the cationic peptide is at a concentration of about 0.01% to about 8% (w/w). In some embodiments, the cationic peptide is at a concentration of about 0.01% to about 9% (w/w). In some embodiments, the cationic peptide is at a concentration of about 0.05% to about 10% (w/w). In some embodiments, the cationic peptide is at a concentration of about 0.1% to about 10% (w/w). In some embodiments, the cationic peptide is at a concentration of about 0.5% to about 10% (w/w). In some embodiments, the cationic peptide is at a concentration of about 1% to about 10% (w/w). In some embodiments, the cationic peptide is at a concentration of about 1.5% to about 10% (w/w). In some embodiments, the cationic peptide is at a concentration of about 2% to about 10% (w/w). In some embodiments, the cationic peptide is at a concentration of about 2.5% to about 10% (w/w). In some embodiments, the cationic peptide is at a concentration of about 1% to about 2% (w/w). In some embodiments, the cationic peptide is at a concentration of about 1% to about 3% (w/w). In some embodiments, the cationic peptide is at a concentration of about 1% to about 4% (w/w). In some embodiments, the cationic peptide is at a concentration of about 1% to about 5% (w/w). In some embodiments, the cationic peptide is at a concentration of about 1% to about 6% (w/w). In some embodiments, the cationic peptide is at a concentration of about 1% to about 7% (w/w). In some embodiments, the cationic peptide is at a concentration of about 1% to about 8% (w/w). In some embodiments, the cationic peptide is at a concentration of about 1% to about 9% (w/w). In some embodiments, the cationic peptide is at a concentration of about 1.5% to about 2% (w/w). In some embodiments, the cationic peptide is at a concentration of about 1.5% to about 2.5% (w/w). In some embodiments, the cationic peptide is at a concentration of about 1.5% to about 3% (w/w). In some embodiments, the cationic peptide is at a concentration of about 1.5% to about 3.5% (w/w). In some embodiments, the cationic peptide is at a concentration of about 0.5% to about 1.5% (w/w). In some embodiments, the cationic peptide is at a concentration of about 0.5% to about 2% (w/w). In some embodiments, the cationic peptide is at a concentration of about 0.5% to about 2.5% (w/w). In some embodiments, the cationic peptide is at a concentration of about 0.5% to about 5% (w/w). In some embodiments, the cationic peptide is at a concentration of about 0.01% (w/w). In some embodiments, the cationic peptide is at a concentration of about 0.02% (w/w). In some embodiments, the cationic peptide is at a concentration of about 0.03% (w/w). In some embodiments, the cationic peptide is at a concentration of about 0.04% (w/w). In some embodiments, the cationic peptide is at a concentration of about 0.05% (w/w). In some embodiments, the cationic peptide is at a concentration of about 0.06% (w/w). In some embodiments, the cationic peptide is at a concentration of about 0.07% (w/w). In some embodiments, the cationic peptide is at a concentration of about 0.08% (w/w). In some embodiments, the cationic peptide is at a concentration of about 0.09% (w/w). In some embodiments, the cationic peptide is at a concentration of about 0.1% (w/w). In some embodiments, the cationic peptide is at a concentration of about 0.2% (w/w). In some embodiments, the cationic peptide is at a concentration of about 0.3% (w/w). In some embodiments, the cationic peptide is at a concentration of about 0.4% (w/w). In some embodiments, the cationic peptide is at a concentration of about 0.5% (w/w). In some embodiments, the cationic peptide is at a concentration of about 0.6% (w/w). In some embodiments, the cationic peptide is at a concentration of about 0.7% (w/w). In some embodiments, the cationic peptide is at a concentration of about 0.8% (w/w). In some embodiments, the cationic peptide is at a concentration of about 0.9% (w/w). In some embodiments, the cationic peptide is at a concentration of about 1% (w/w). In some embodiments, the cationic peptide is at a concentration of about 1.5% (w/w). In some embodiments, the cationic peptide is at a concentration of about 2% (w/w). In some embodiments, the cationic peptide is at a concentration of 2.3% (w/w). In some embodiments, the cationic peptide is at a concentration of about 2.5% (w/w). In some embodiments, the cationic peptide is at a concentration of about 3% (w/w). In some embodiments, the cationic peptide is at a concentration of about 3.5% (w/w). In some embodiments, the cationic peptide is at a concentration of about 4.0% (w/w). In some embodiments, the cationic peptide is at a concentration of about 4.5% (w/w). In some embodiments, the cationic peptide is at a concentration of about 5% (w/w). In some embodiments, the cationic peptide is at a concentration of about 5.5% (w/w). In some embodiments, the cationic peptide is at a concentration of about 6% (w/w). In some embodiments, the cationic peptide is at a concentration of about 6.5% (w/w). In some embodiments, the cationic peptide is at a concentration of about 7% (w/w). In some embodiments, the cationic peptide is at a concentration of about 7.5% (w/w). In some embodiments, the cationic peptide is at a concentration of about 8% (w/w). In some embodiments, the cationic peptide is at a concentration of about 8.5% (w/w). In some embodiments, the cationic peptide is at a concentration of about 9% (w/w). In some embodiments, the cationic peptide is at a concentration of about 9.5% (w/w). In some embodiments, the cationic peptide is at a concentration of about 10% (w/w).

Solvents useful in the present compositions are well known in the art and include without limitation water, glycerin, propylene glycol, mineral oil, isopropanol, ethanol, and methanol. In some embodiments, the solvent is glycerin, propylene glycol or mineral oil, at a concentration ranging from about 0.1% to about 20%, about 5% to about 15%, and about 9% to 11%. In other embodiments, the solvent is water or ethanol, preferably at a concentration up to about 99%, up to about 90%, and up to about 85%. Unless otherwise indicated, all percentages are on a w/w basis. In yet other embodiments, the solvent is at least one of water, glycerin, propylene glycol, mineral oil, isopropanol, ethanol, and methanol. In some embodiments, the solvent is glycerin or propylene glycol, mineral oil and ethanol. In other embodiments, the solvent is glycerin and ethanol. In yet other embodiments, the solvent is glycerin and water. One embodiment is a composition comprising the cationic peptide, a viscosity-increasing agent, a solvent, wherein the solvent comprises at least one of water at a concentration up to 99%, glycerin at a concentration up to 20%, propylene glycol at a concentration up to 20%, ethanol at a concentration up to 99%, and methanol at a concentration up to 99%.

Another useful pharmaceutical excipient of the present invention is a viscosity-increasing agent. In certain embodiments, the cationic peptide compositions of the present invention include a viscosity-increasing agent, including without limitation carbomer homopolymer, dextran, polyvinylpyrrolidone, methylcellulose, carboxymethyl cellulose, hydroxyethyl cellulose, hydroxypropyl methylcellulose, and hydroxypropyl cellulose, and combinations thereof. In some embodiments, the viscosity-increasing agent is hydroxyethyl cellulose or hydroxypropyl methylcellulose, at a concentration ranging from about 0.5% to about 5%, from about 1% to about 3%, and from about 1.3% to about 1.7%. In yet other preferred embodiments, the cationic peptide compositions have a first viscosity increasing agent, such as carbomer homopolymer, hydroxyethyl cellulose, hydroxypropyl methylcellulose, dextran, or polyvinylpyrrolidone, and a second viscosity-increasing agent such as carbomer homopolymer, hydroxyethyl cellulose, hydroxypropyl methyl cellulose, dextran, or polyvinylpyrrolidone. When used as either a first or second viscosity-increasing agent, dextran and polyvinylpyrrolidone are preferably used at a concentration ranging from about 0.1% to about 5% and more preferably from about 0.5% to about 1%. In one preferred embodiment, the first viscosity increasing agent is hydroxyethyl cellulose at a concentration up to 3% and the second viscosity-increasing agent is hydroxypropyl methylcellulose at a concentration up to 3%. As is known in the art, the amount of viscosity-increasing agent may be increased to shift the form of the composition from a liquid to a gel to a semi-solid form. Thus, the amount of a viscosity-increasing agent used in a formulation may be varied depending on the intended use and location of administration of the peptide compositions provided herein.

In certain applications, it may be desirable to maintain the pH of the cationic peptide composition contemplated by the present invention within a physiologically acceptable range and within a range that optimizes the activity of the peptide or analog or derivative thereof. The cationic peptides of the present invention function best in a composition that is neutral or somewhat acidic, although the peptides will still have antiviral, anti-inflammatory, and/or immune enhancing activity in a composition that is slightly basic (i.e., pH 8). Accordingly, a composition comprising the cationic peptide, a viscosity-increasing agent, and a solvent, may further comprise a buffering agent. In certain embodiments, the buffering agent comprises a monocarboxylate or a dicarboxylate, and more specifically may be acetate, benzoate, fumarate, lactate, malonate, sorbate, succinate, or tartrate. In some embodiments, the buffering agent comprises benzoate. In some embodiments, the cationic peptide composition comprising the buffering agent has a pH ranging from about 3 to about 8, and more preferably from about 3.5 to 7. In some embodiment, the buffering agent is at a concentration ranging from about 1 mM to about 200 mM, from about 2 mM to about 20 mM, and about 4 mM to about 6 mM.

Other optional pharmaceutically acceptable excipients are those that may, for example, aid in the administration of the formulation (e.g., anti-irritant, polymer carrier, adjuvant) or aid in protecting the integrity of the components of the formulation (e.g., antioxidants and preservatives. Typically, a 1.0% cationic peptide composition may be stored at 2° C. to 8° C. In certain embodiments, the composition comprising a cationic peptide, a viscosity-increasing agent, and a solvent, may further comprise a humectant, (e.g., sorbitol and the like), or a preservative, (e.g., benzoic acid, benzyl alcohol, phenoxyethanol, methylparaben, propylparaben, and the like). In certain circumstances, the cationic peptide or analog or derivative thereof may itself function as a preservative of the final therapeutic composition. For example, a preservative is optional in the gel formulations described herein because the gels may be sterilized by autoclaving and, furthermore, show the surprising quality of releasing (i.e., making bioavailable) the cationic peptide at a more optimal rate than other formulations, such as a cream. In addition, particular embodiments may have in a single formulation a humectant, a preservative, and a buffering agent, or combinations thereof. Therefore, in some embodiments, the composition comprises a cationic peptide, a viscosity-increasing agent, a solvent, a humectant, and a buffering agent. In some embodiments, the composition comprises a cationic peptide, a viscosity-increasing agent, a buffering agent, and a solvent. In some embodiments, the composition comprises a cationic peptide, a buffering agent, and a solvent. Each of the above formulations may be used to treat or prevent viral infection or to reduce the viral load in a subject or at a target site.

In yet other embodiments, the composition is in the form of an ointment comprising a cationic peptide (preferably in an amount sufficient to treat or prevent an infection) and an oleaginous compound. For example, oleaginous compound may be petrolatum. In one embodiment, the oleaginous compound is present at a concentration ranging from about 50% to about 100%, more preferably from about 70% to about 100%, even more preferably from about 80% to about 100%, and most preferably from about 95% to about 100%. In certain other embodiments, the ointment composition may further comprise at least one emollient. The emollients may be present at a concentration ranging from about 1% to about 40%, more preferably from about 5% to about 30%, and more preferably from about 5% to about 10%. In some embodiments, the emollient may be mineral oil, cetostearyl alcohol, glyceryl stearate, and a combination thereof. In another aspect the composition is in the form of a semi-solid emulsion (e.g., a cream) comprising a cationic peptide, a solvent, a buffering agent, at least one emollient, and at least one emulsifier. In some embodiments, the semi-solid emulsion or cream further comprises at least one of a humectant (e.g., sorbitol and/or glycerin), an oleaginous compound (e.g., petrolatum), a viscosity increasing agent (e.g., dextran, polyvinylpyrrolidone, hydroxyethyl cellulose, and/or hydroxypropyl methylcellulose), an anti-oxidant (e.g., butylated hydroxytoluene and preferably at a concentration ranging from about 0.01% to about 0.1%), a preservative (e.g., benzoic acid, benzyl alcohol, phenoxyethanol, methylparaben, propylparaben, or a combination thereof), or a combination thereof. In some embodiments, the emollient may be one or more of stearyl alcohol, cetyl alcohol, and mineral oil. In certain other preferred embodiments, the emulsifiers may be one or more of stearyl alcohol, cetyl alcohol, polyoxyethylene 40 stearate, and glyceryl monostearate. In some embodiments, the emulsifier is present at a concentration ranging from about 1% to about 20%, from about 5% to about 10%, and from about 1% to about 1.5%. As noted above, the function of each of these emulsifiers and emollients is not mutually exclusive in that an emollient may function as an emulsifier and the emulsifier may function as an emollient, depending on the particular formulation, as is known in the art and is described herein. In some embodiments the solvent comprises water and the like, and the buffering agent comprises a monocarboxylate or dicarboxylate and the like, as described herein.

A subject suitable for treatment with the cationic peptides of the present invention may be identified by well-established indicators of risk for developing a disease or well-established hallmarks of an existing disease. For example, indicators of an infection include coughing, sore throat, chest infection, sneezing, runny nose, blocked nose, fever, inflammation, diarrhea, dehydration, rash, blisters, welts, raised bumps, cold sores, viral warts, itching, headache, fatigue, body ache, cramping, abdominal pain, vomiting, positive cultures, positive blood tests for antibodies to the virus or for viral antigens, PCR detection of viral genetic material, detection of viral particles by electron microscopy, and the like. Infections that may be treated with the cationic peptides provided by the present invention include without limitation those caused by or due to viruses, whether the infection is primary, secondary, opportunistic, or the like. Examples of viruses that may be treated with the cationic peptides of the present invention include coronavirus (including, but not limited to, SARS coronavirus), coxsackievirus, cytomegalovirus, echovirus, enterovirus, Epstein-Barr virus, influenza virus, hepatotropic viruses (including, but not limited to, Hepatitis A, Hepatitis B, Hepatitis C, Hepatitis D, and Hepatitis E,), human immunodeficiency virus (HIV), human papillomavirus (HPV), herpes simplex virus (including, but not limited to, HSV-1 and HSV-2), poxvirus, norovirus, rabies virus, rhinovirus, rotavirus, Rous sarcoma virus (RSV), Varicella zoster virus, parvovirus or West Nile virus. Infections include, but are not limited to, acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection, gingivostomatitis, tonsillitis & pharyngitis, keratoconjunctivitis, herpes labialis, cold sores, aseptic meningitis, Cytomegalic inclusion disease, Kaposi sarcoma, AIDS, influenza, Reye syndrome, measles, post-infectious encephalomyelitis, mumps, hyperplastic epithelial lesions (common, flat, plantar and anogenital warts, laryngeal papillomas, epidermodysplasia verruciformis), giant condylomata (Buschke-Löwenstein tumors), cervical intraepithelial neoplasia, cervical carcinoma, vulvar intraepithelial neoplasia, vulvar cancer, penile carcinoma, anal intraepithelial neoplasia, anal carcinoma, actinic keratosis, squamous cell carcinomas, conjunctival papillomas and carcinomas, oral leukoplakia and carcinomas, croup, pneumonia, common cold, poliomyelitis, rabies, bronchiolitis, influenza-like syndrome, severe bronchiolitis with pneumonia, german measles, congenital rubella, varicella (chicken pox) and herpes zoster (shingles), BK virus infection, bolivian hemorrhagic fever, chikungunya, Colorado tick fever, Crimean-Congo hemorrhagic fever, cytomegalovirus infection, dengue fever, ebola hemorrhagic fever, enterovirus infection, erythema infectiosum (Fifth disease), exanthema subitum (Sixth disease), hand, foot and mouth disease, hantavirus pulmonary syndrome, heartland virus disease, human bocavirus infection, human metapneumovirus infection, human parainfluenza virus infection, lassa fever, Marburg hemorrhagic fever, Middle East respiratory syndrome, monkeypox, progressive multifocal leukoencephalopathy, Rift valley fever, Severe Acute Respiratory Syndrome (SARS), subacute sclerosing panencephalitis, Venezuelan equine encephalitis, Venezuelan hemorrhagic fever, West Nile fever and Yellow fever.

In one aspect, the present invention provides compositions and methods for treating virally-induced skin diseases. Examples of skin diseases that may be treated with the cationic peptides of the present invention include warts, anogenital warts, benign or malignant tumors of the skin and/or mucosa which are caused by papilloma viruses, for example verrucae plantares, verrucae vulgares, verrucae planae juveniles, epidermodysplasia verruciformis, Condylomata acuminata, Condylomata plana, bowenoid papulosis, papillomas on the larynx and oral mucosa, focal epithelial hyperplasia, herpes labialis, Kaposi's sarcoma, varicella and shingles. These viral skin diseases and/or tumor diseases are caused by at least one papilloma virus or viruses, in particular human papilloma viruses, such as HPV 1, 2, 3, 4, 5, 6, 8, 9, 11, 12, 13, 14, 15, 16, 17, 18, 19-29, 31, 32, 34, 36-38, 46-50, 56, 58, by at least one herpes virus or herpes viruses, such as herpes simplex virus 1, herpes simplex virus 2, varicella zoster virus or human herpes virus, such as 1, 2, 3, 4, 7 or 8. In some embodiments, a cationic peptide composition of the invention is administered to a subject to treat or ameliorate the symptoms associated with a virally-induced skin disease. In some embodiments, the administration of the cationic peptide reduces the viral load. In some embodiments, a cationic peptide composition of the invention is administered to a subject to treat or ameliorate the symptoms associated with an HPV-induced skin disease. HPV-induced skin diseases may include ordinary warts, plantar warts, genital warts, anal warts, or tumors of the skin and/or mucosa. In some embodiments, the tumors of the skin and/or mucosa are benign. In some embodiments, the tumors of the skin and/or mucosa are malignant. In some embodiments, the HPV-induced skin disease is non-melanoma skin cancer. In some embodiments, the cancer is squamous cell skin cancer.

### EXAMPLES

In order that this invention be more fully understood, the following examples are set forth. These examples are for the purpose of illustration only and are not to be construed as limiting the scope of the invention in any way.

### Experimental Conditions

### PBMC Culture Preparation

Peripheral Blood Mononuclear Cells (PBMCs) were isolated from sodium heparinized blood samples from healthy donors using SEPMATE with LYMPHOPREP (STEMCELL TECHNOLOGIES) based on ficoll density centrifugation. Primary cell cultures were prepared in RPMI including 5% autologous plasma. Isolated PBMC cells were cultured in 96-well plates at a density of 0.5×10⁶cells/well at 37° C., under humidified 5% CO₂.

### Preparation and Storage of Experimental Compounds

LL-37 was synthesized by ANASPEC. Omiganan was commercially prepared by chemical synthesis. LL-37 and omiganan stocks were dissolved in sterile H₂O and stored at −20° C. PolyI:C(HMW), PolyI:C(HMW)/Lyovec and Imiquimod were purchased from INVIVOGEN and handled according to the supplier's instructions. PolyI:C (HMW)/Lyovec was resuspended in endotoxin-free water and stored at −20° C. PolyI:C(HMW) (not complexed to Lyovec) was resuspended in endotoxin-free water, heated to 65° C.-70° C. for 10 minutes to dissolve and stored at −20° C. Imiquimod was resuspended in endotoxin-free water and aliquots were stored at −20° C.

### IL-1β, TNFα, IL-6 and IL-8 Detection

IL-1β, TNFα, IL-6 and IL-8 were measured simultaneously with a fixed electrochemoluminescence platform based on the sandwich ELISA principle of capture and detection antibodies, produced by MESO SCALE DISCOVERY (MSD, MESO SCALE QUICKPLEX SQ120). The applied assay was the V-PLEX PLUS pro-inflammatory cytokine panel II, including IL-1β, TNFα, IL-6 and IL-8. This assay includes manufacturer-provided control samples with indicated nominal values for comparison. These lyophilized controls, i.e. freeze-dried controls for IL-1β, TNFα, IL-6, IL-8, were prepared freshly at the time of use. QC samples were prepared from the calibrator at 3 different concentrations along the standard curve as multiple aliquots from a single batch and maintained at −20° C. Low, medium and high concentration controls were included in every multiplex assay run to control intra- and inter-assay variation. V-PLEX panels are specific for the detection of human chemokines and cytokines, and combinations have been validated by the manufacturer, to ensure no cross-reactivity.

### Pan-IFNα Detection

IFNα was quantified by a sandwich ELISA based on specific capture and detection antibodies for various matrices (MABTECH), according to the manufacturer's instructions. QC samples were prepared from the calibrator at 3 different concentrations along the standard curve as multiple aliquots from a single batch and maintained at −20° C.

### IFNβ Detection

IFNβ was quantified by a sandwich ELISA based on specific capture and detection antibodies for various matrices (PBL ASSAY SCIENCE), according to the manufacturer's instructions. QC samples were prepared from the calibrator at 3 different concentrations along the standard curve as multiple aliquots from a single batch and maintained at −20° C. freezer.

### Data Handling

In accordance with the FDA Guidance for Industry for bioanalytical methods, the quantitative limits were based on the actual performance of calibration curves in a given experiment.

In order to capture the broadest range of sample values for a given analyte, samples series from a single subject (nr 1) were first analyzed to estimate an appropriate dilution factor for subsequent analyses. All culture supernatants were measured undiluted or diluted in the manufacturer-provided diluent. The limits of quantification are listed below as a function of the assay calibration range and the specific sample dilution factor applied. See Table 2.

TABLE 2
Cytokine Detection Limits

| pan-IFNα | LLOQ | ULOQ |
|---|---|---|
| Calibration range (pg/mL) | 12.5 | 800 |
| Initial set-up experiment | 25 | 1600 |
| Experiments utilizing poly I:C and controls¹ | 50 | 3200 |
| Experiments utilizing imiquimod² | 70 | 4480 |
| IFNβ | LLOQ | ULOQ |
| Calibration range (pg/mL) | 62.5 | 4000 |
| All experiments | 62.4 | 4000 |
| TNFα | LLOQ | ULOQ |
| Calibration range | 0.08 | 315 |
| Initial set-up experiment | 0.8 | 3150 |
| Experiments utilizing polyI:C and controls* | 0.16 | 630 |
| Experiments using imiquimod^{†} | 0.4 | 1575 |
| IL-6 | LLOQ | ULOQ |
| Calibration range (pg/mL) | 0.19 | 789 |
| Initial set-up experiment | 1.9 | 7890 |
| Experiments utilizing polyI:C and controls* | 0.38 | 1578 |
| Experiments using imiquimod^{†} | 0.95 | 3948 |
| IL-1β | LLOQ | ULOQ |
| Calibration range (pg/mL) | 0.15 | 620 |
| Initial set-up experiment | 1.5 | 6200 |
| Experiments utilizing polyI:C and controls* | 0.3 | 1240 |
| Experiments using imiquimodt | 0.75 | 3100 |
| IL-8 | LLOQ | ULOQ |
| Calibration range (pg/mL) | 0.13 | 519 |
| Initial set-up experiment | 1.3 | 5190 |
| Experiments utilizing polyI:C and controls* | 0.26 | 1038 |
| Experiments using imiquimod^{†} | 0.65 | 2595 |

¹Includes subject 1 with imiquimod
²Excludes subject 1

All data were reported, including data outside the quantification range (<LLOQ, >ULOQ). While the extrapolated data outside the quantification range cannot be guaranteed for accuracy and precision, it does provide additional insight into general trends that may be observed in the extrapolated data range. Data points marked as ‘ND’ (not detected) or ‘NaN’ (not a number) in the data listings were below the LLOQ, but too low to be extrapolated.

Data from duplicate cultures were reported as the mean value of the two cultures, with the accompanying (%) CV. No strict limit was applied to the CV of duplicate cultures, and data for cultures exhibiting a CV greater than 40% were also reported. Overall, assay performance was very good and few cultures displayed high CVs, generally at the extreme ends of the responses only.

### Example 1 Qualification of the polyI:C PBMC Challenge

Exposure of cells to naked polyI:C generally leads to uptake in the endosomal compartments and stimulation of toll-like receptor 3 (TLR3) pathways. When polyI:C is formulated into liposomal vesicles (Lyovec-complexed polyI:C), it is also transfected into the cytoplasm, engaging RIG-1/Mda5 responses.

This experiment was conducted to confirm the optimal conditions for polyI:C stimulation of primary PBMCs and to demonstrate dynamic shifts in the dose response curves dependent on the formulation of polyI:C.

The PBMC cultures were incubated with varying doses of polyI:C (HMW)/Lyovec or polyI:C(HMW) as per the conditions described: (a) polyI:C (10000, 2000, 400, 80, 16 ng/mL); (b) PolyI:C(HMW)/Lyovec (1000, 333, 111, 37, 12.3 ng/mL); and (c) Unstimulated. Cells were incubated for 24 hrs in duplicate cultures and the readouts for IL-1β, TNFα, IL-6 and IL-8 (multiplex), pan-IFNα and IFNβ (ELISA) were measured once. Concentration-response curves were constructed for polyl:C in PBMC cultures, in the presence and absence of transfection carrier (Lyovec).

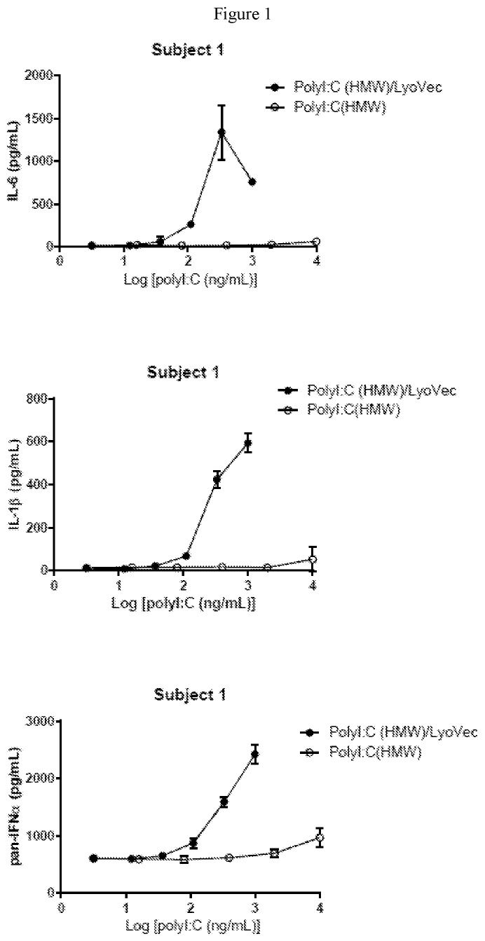

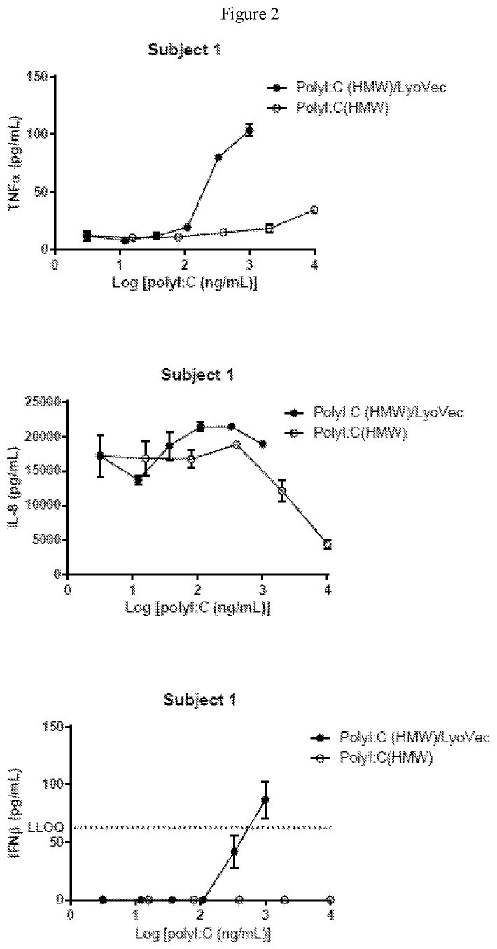

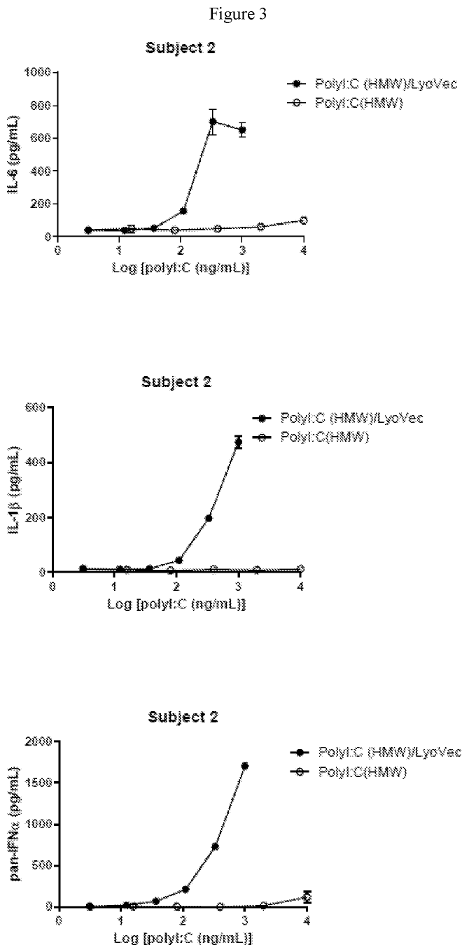

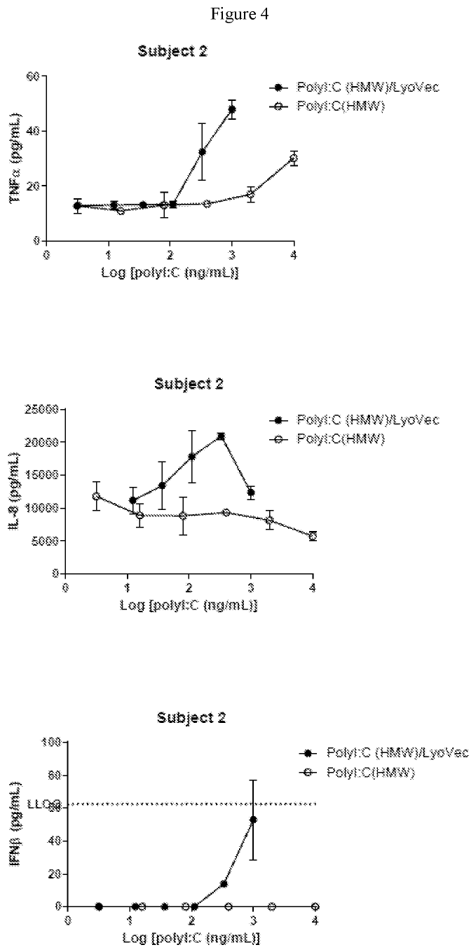

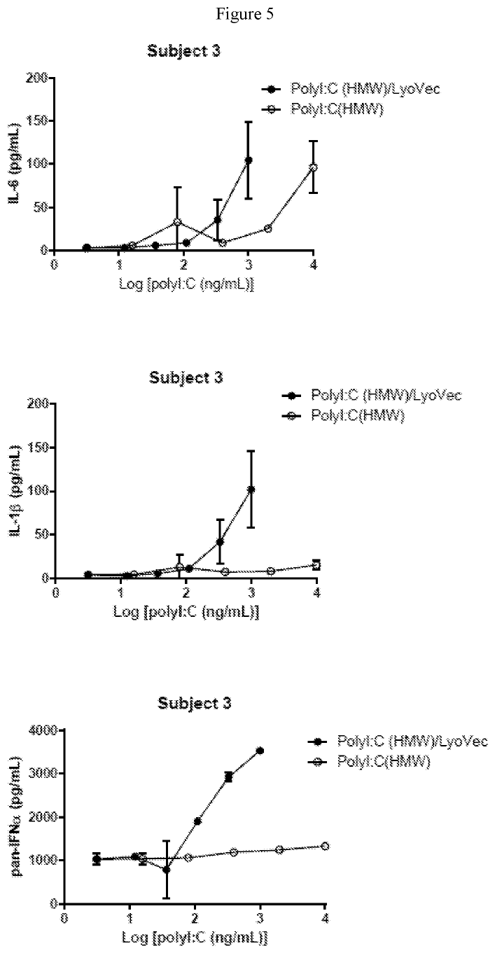

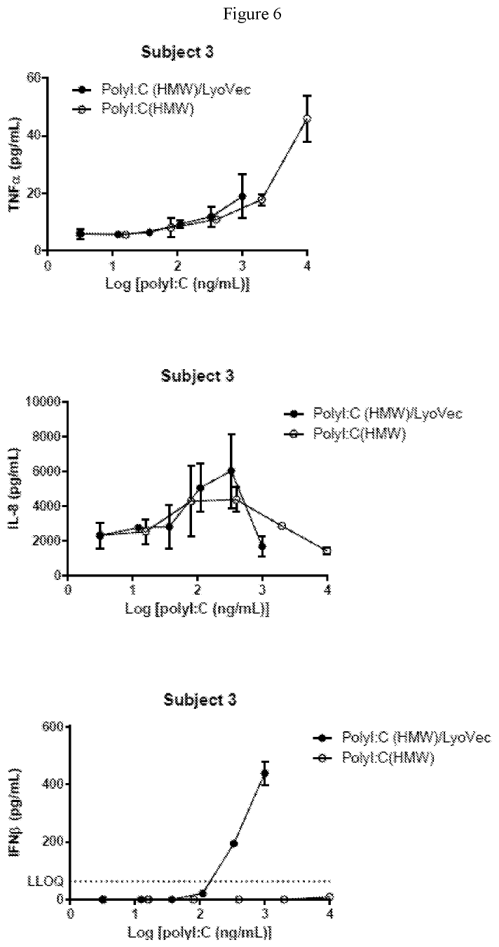

These experiments demonstrate dynamic shifts in the dose response curves depending on which polyI:C formulation is used (see FIGS. 1-6). PolyI:C induces a mild PBMC response in the absence of the transfection carrier Lyovec (see FIGS. 1-6). By contrast, substantial polyI:C-driven responses were observed in the presence of Lyovec transfection carrier, which induced a left-shift in the dose response curve and provided a proof-of-principal for carrier-enhanced delivery of the trigger to the endosomal compartment (TLR3) and the cytosol (RIG-1 engagement). These data demonstrate that the polyI:C PBMC challenge is a suitable model for exploration of the potential antiviral effects of the cationic peptides.

### Example 2 Effects of Omiganan in PBMC Cultures

The effect of omiganan and LL-37 on polyI:C- and imiquimod-induced cytokine responses (TLR3 and TLR7, respectively) was studied in PBMC cultures. Briefly, PBMC primary cultures were prepared from 4 healthy donors (2 males, 2 females) as described above. The cultures were incubated with varying doses of polyI:C alone, polyI:C with omiganan, polyI:C with LL-37, imiquimod alone, imiquimod with omiganan or imiquimod with LL-37 as per the conditions described: (a) polyI:C (50000, 10000, 2000, 400, 80 ng/mL); (b) polyI:C (50000, 10000, 2000, 400, 80 ng/mL) and 5 or 25 μg/mL omiganan; (c) polyI:C (50000, 10000, 2000, 400, 80 ng/mL) and 5 or 25 μg/mL LL-37; (d) imiquimod (50000, 10000, 2000, 400, 80 ng/mL); (e) imiquimod (50000, 10000, 2000, 400, 80 ng/mL) and 5 or 25 μg/mL omiganan; (f) imiquimod (50000, 10000, 2000, 400, 80 ng/mL) and 5 or 25 μg/mL LL-37; (g) Unstimulated (negative control); and (h) polyI:C (HMW)/Lyovec (300 ng/mL) (positive control). Cells were incubated for 24 hrs including a 30 min pre-treatment with the peptides (omiganan or LL-37). The cultures were prepared in duplicate and the readouts for pan-IFNα and IFNβ (ELISA) were measured once. Five-point concentration-response curves were constructed for the various treatment conditions in the PBMC cultures, freshly isolated from 4 subjects.

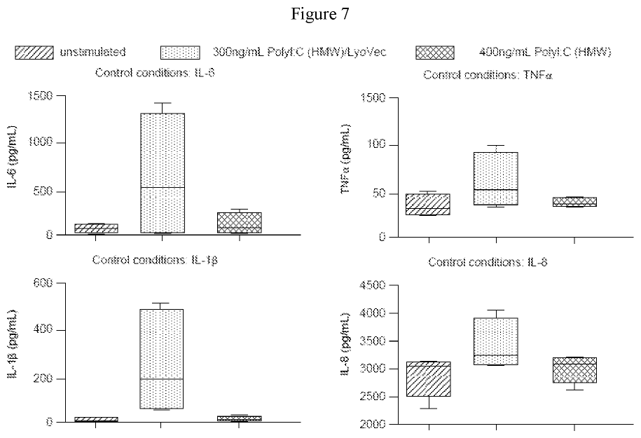

The amount of IL-6, TNFα, IL1β and IL-8 released in response to control conditions (unstimulated, polyI:C(HMW)/Lyovec (300 ng/mL) and polyI:C(HMW) (400 ng/mL) are provided in FIG. 7.

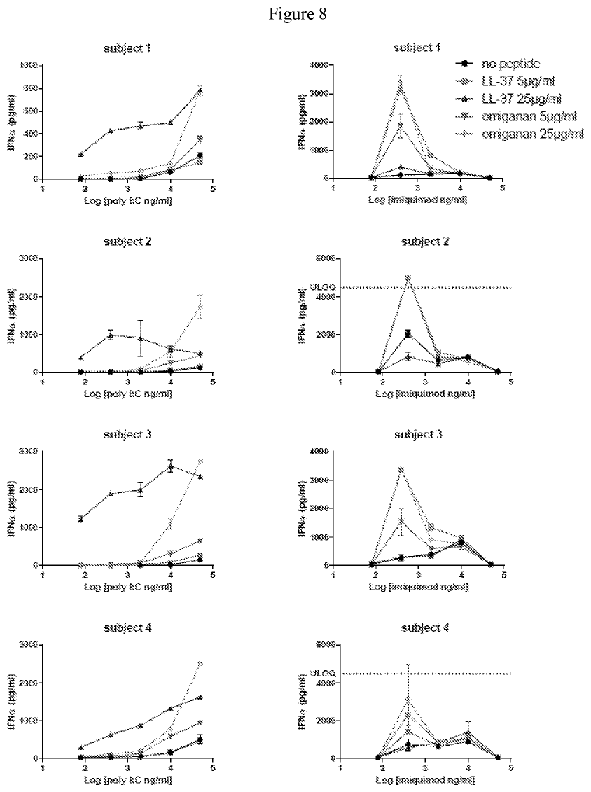

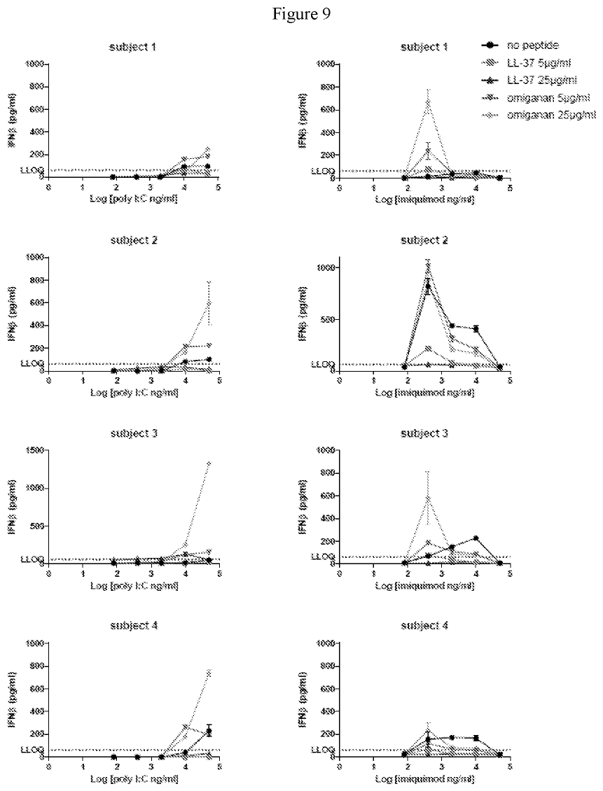

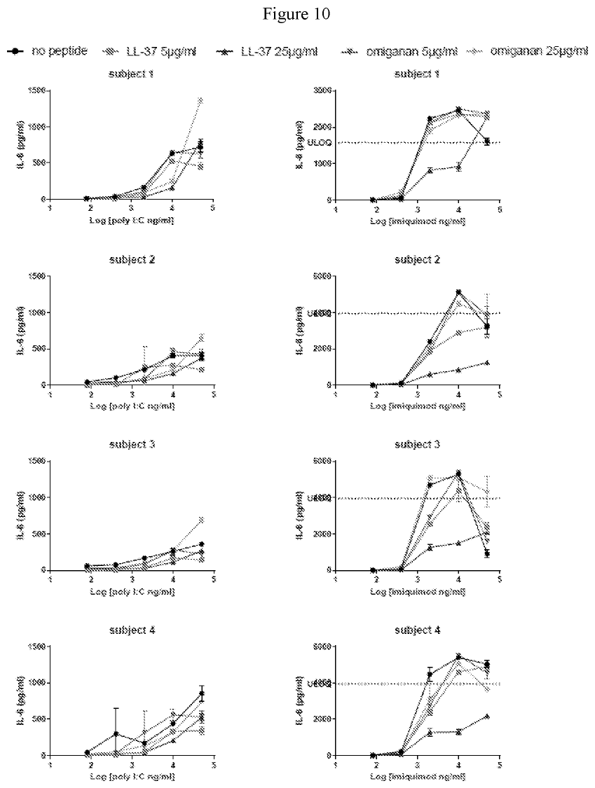

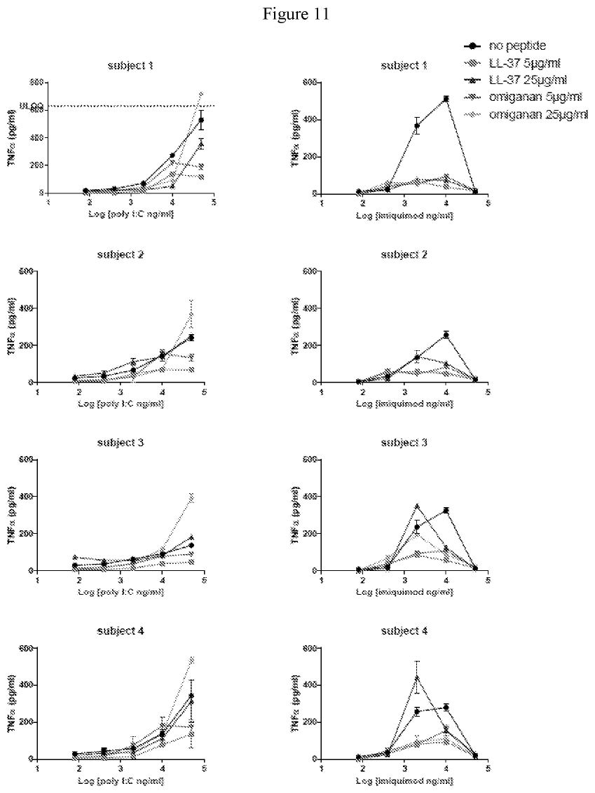

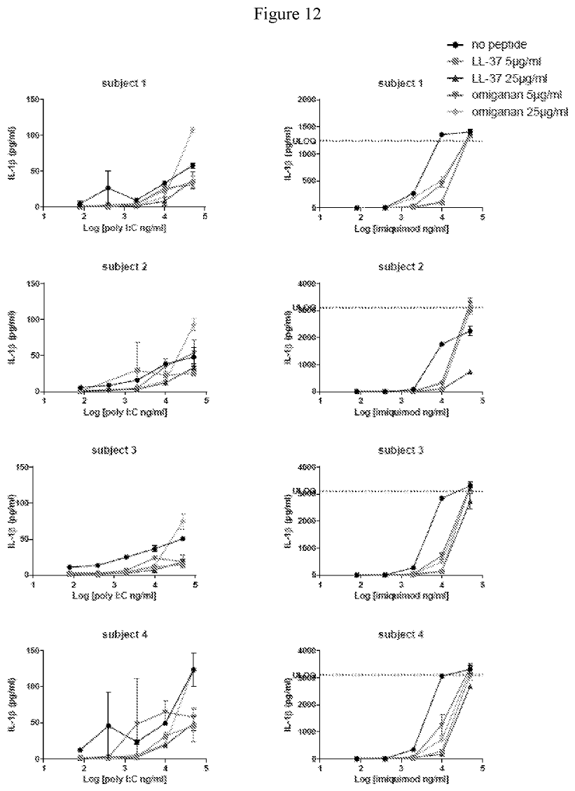

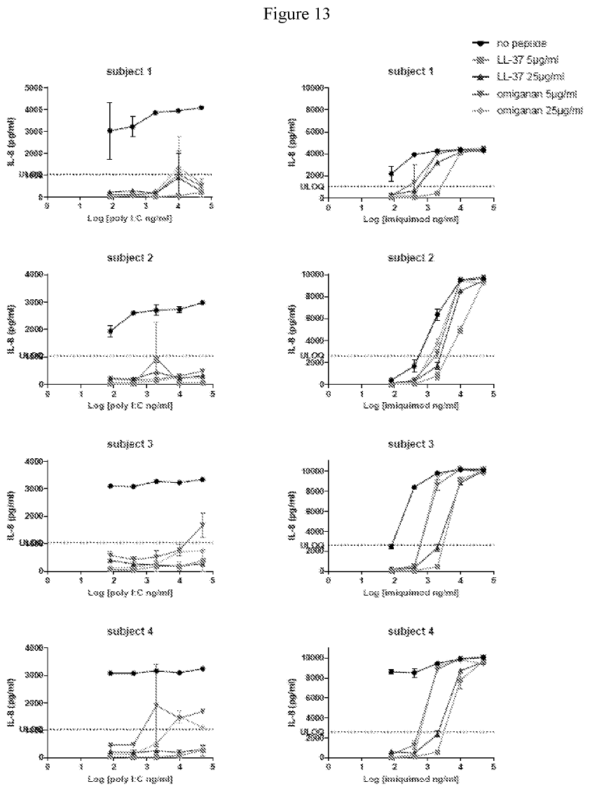

In the presence of imiquimod (a TLR7 trigger), LL-37 and omiganan induced strong interferon responses. See FIGS. 8 and 9. Similarly, LL-37 and omiganan enhanced interferon responses induced by polyI:C stimulation (a TLR3/RIG-1/Mda5 trigger). See FIGS. 8 and 9. While LL-37 and omiganan inhibited TNFα and IL-6 responses to imiquimod (TLR7), they did not significantly affect TNFα and IL-6 responses to polyI:C (TLR3/RIG-1/Mda5). See FIGS. 10 and 11. LL-37 and omiganan inhibited IL-1(3 and IL-8 responses to imiquimod (TLR7), as demonstrated by the right-shift of the imiquimod concentration response curves. See FIGS. 12 and 13. LL-37 and omiganan strongly inhibited spontaneous and polyI:C-driven (TLR3/RIG-1/Mda5-driven) IL-8 release, but did not significantly affect polyI:C-driven IL-1β release. See FIGS. 12 and 13. This inhibition of TNFα, IL-6, IL1β and IL-8 by LL-37 and omiganan demonstrates the immune-suppressive effects of these cationic peptides.

### Example 3 Effects of Omiganan on Viral Uptake

The effect of omiganan on viral uptake was studied in 293TT cultures. Pseudovirus (PsV) of HPVS was prepared using method described by Buck et al, (Journal of Virology, January 2004; 751-757) and used as a model HPV in this study. The PsV stocks were prepared by co-transfecting human 293TT cells with HPVS viral plasmid p5Shell containing codon-modified papillomavirus capsid genes for L1 and L2 proteins, and pcLucF reporter plasmid containing the luciferase gene for detecting viral infection. The cell lysates containing the capsidized virus was used as the PsV stock. Optimal PsV dilution factor was determined based on the luciferase activity in 293TT cells infected with serially diluted HPVS PsV. The optimal luciferase activity was found around 1:3000 to 1:6000 PsV dilution (data not shown). Therefore, the PsV at these dilutions was used in the HPV inhibition assay.

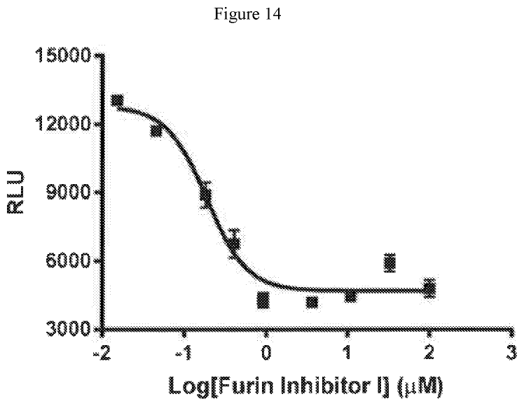

For the inhibition assay, 293TT cells were incubated with PsV stock in the presence of omiganan, and luciferase activity was detected using the Steady-Glo Luciferase kit from Promega as a measure of the viral infection. Data were collected on a Molecular Devices Flexstation 3 Microplate Reader using Softmax Pro v5.4.4. Data analysis was performed on Prism software using four parameter logistic model. Omiganan concentration versus luciferase activity (RLU) was plotted and the IC₅₀ concentration was calculated. Furin Inhibitor I was used as a positive control for HPVS PsV inhibition since it blocks the viral entry into host cells. As shown in FIG. 14, this Furin Inhibitor I demonstrated potent inhibition on the HPV PsV with an IC₅₀ of 0.2 μM as expected.

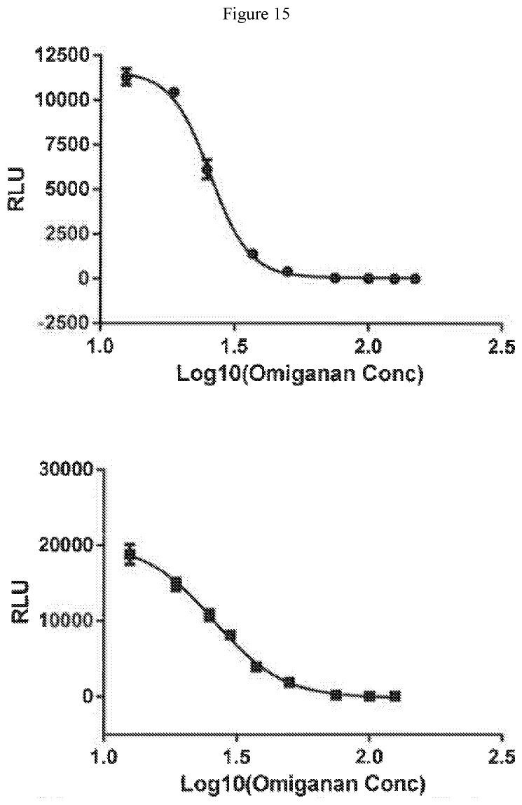

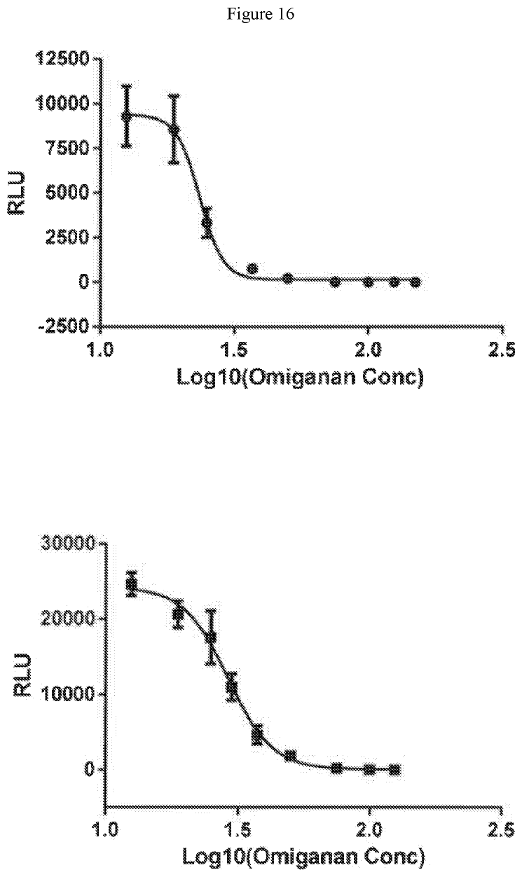

In the HPV inhibition test, the PsV stock diluted at 1:3000 and 1:6000 was incubated with the 293TT cells in the presence of 0-150 μg/mL omiganan followed by measurement of the cellular luciferase activity (RLU). Two runs were performed for each dilution level. As shown in FIGS. 15 and 16, omiganan demonstrated dose-dependent inhibition on HPV infection with similar IC₅₀ values ranging from 23.5 μg/mL to 29.1 μg/mL at the two PsV dilutions in the two runs.

### Example 4 Effects of Topical Omiganan in Patients with External Anogenital Warts

A randomized, double-blind, placebo-controlled study to assess the pharmacodynamics, safety/tolerability and efficacy of omiganan in patients with external anogenital warts was conducted. 24 patients aged 18 years or older with clinically diagnosed external anogenital warts were enrolled. Eligible patients were randomized on a 2:1 basis to two treatment arms: 2.5% omiganan pentahydrocholoride gel or vehicle, i.e. placebo gel. The gel was applied topically once daily for 12 weeks.

The treatment period was from day 0 till day 84. During the treatment period subjects visited the clinical unit on days 0, 14, 28, 56 and 84. A fixed dose of omiganan or placebo was applied once daily. Two follow up visits were scheduled on days 126 and 168. During study execution, vital signs were measured on days 0, 14, 28, 56 and 84. Pharmacodynamic assessments were performed on day 0 (pre-dose), 14, 28, 56 and 84, respectively. Punch biopsies were performed at the screening and on days 56 and 168. Subjects were dosed 84 consecutive days with approximately 24 hours between each treatment.

Omiganan or placebo (vehicle) was administered to the subjects. Each subject was administered a dose of either omiganan or the vehicle comparator product on eighty-four (84) consecutive days. Subjects were instructed to apply a fingertip unit of gel (approximately 10 mg). Omiganan is prepared as a lyophilized powder of the pentahydrochloride salt. The molecular formula is C₉₀H₁₂₇N₂₇O₁₂. HCl*H₂O. The molecular weight of omiganan is 1779.2 Dalton, while the pentahydrochloride salt has a molecular weight of 1961.5 Dalton. The dosage in the study was 2.5% once daily.

Any other therapy for external anogenital warts (topical treatment, laser therapy, cryotherapy, photodynamic therapy and surgical excision) was not allowed from 28 days prior to the first study drug administration, during the study and until the 3 month follow up assessment was completed.

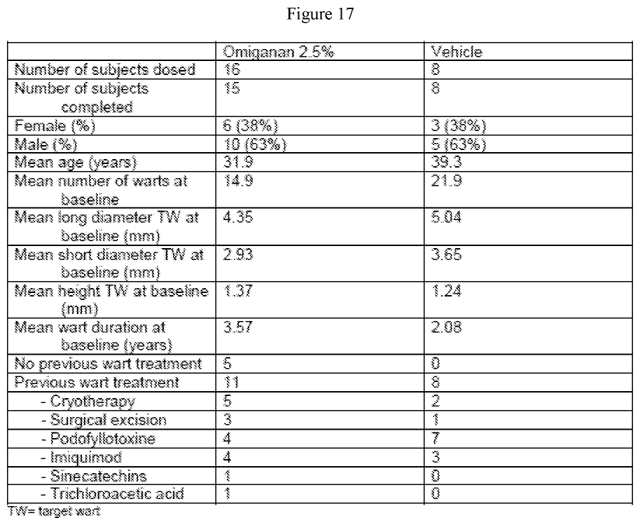

No important differences in the disease and anogenital wart characteristics of the two treatment groups were noted prior to the start of the study (FIG. 17). The analysis population consisted of 23 subjects.

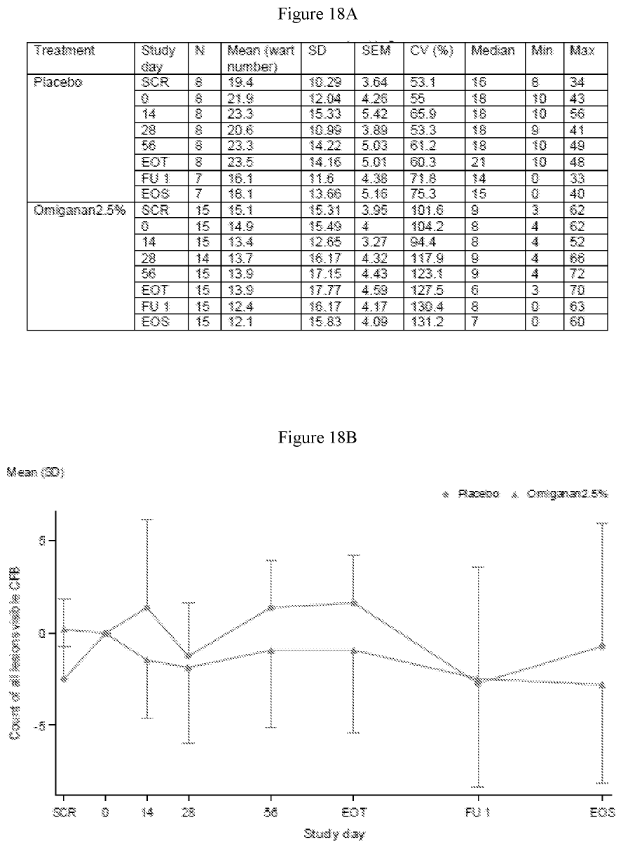

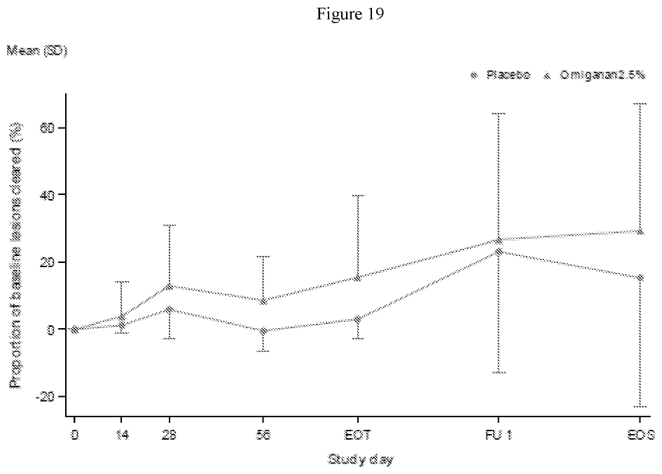

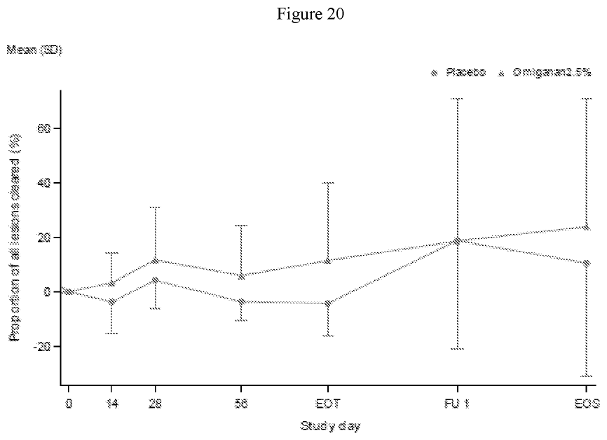

Wart count and wart clearance. The warts were counted at every study visit. FIGS. 18A and 18B show the change in value of the count of the target warts over time per treatment. In the omiganan 2.5% group, there was a change in mean lesion count from 14.9 warts (SD15.5) pre-dose to 13.9 warts (SD 17.8) at end of treatment (EOT) to 12.1 warts (SD15.8) at end of study (EOS). No significant difference (p-value=0.1672) was observed between the omiganan 2.5% group and the vehicle. Wart clearance was derived from the count of all warts present at baseline. In FIG. 19, the proportion of baseline lesions cleared is shown. At EOT, in the omiganan group, a mean of 15.5% of baseline warts was cleared compared to 2.9% in the vehicle group (p-value=0.2500). At EOS, in the omiganan group a mean of 29.3% of baseline warts was cleared compared to 15.3% in the vehicle group (p-value=0.3918). FIG. 20 shows the proportion of all lesions cleared. AT EOT, in the omiganan group a mean of 11.4% of all warts was cleared compared to -4.34% in the vehicle group (p-value=0.1879). At EOS, in the omiganan group, a mean of 23.82% of baseline warts was cleared compared to 10.42% in the vehicle group (p-value=0.2891).

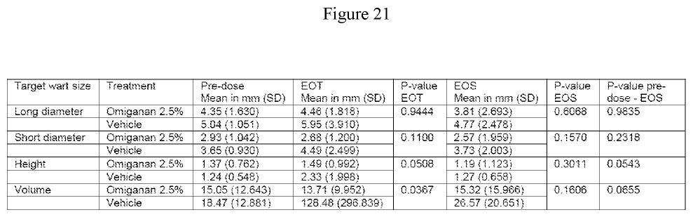

Wart size. Wart size was measured during every visit. The change in mean of the long and short diameter, height and volume measured is presented in FIG. 21 along with the p-values. The mean volume of the target warts in the omiganan group was significantly lower (p-value=0.0367) than in the vehicle group after the treatment period (at EOT visit). When comparing the volume of the warts in the omiganan and the vehicle group over the entire study period (pre-dose till EOS), but there was a trend to reduction in the omiganan group (p=0.0655). There was also a clear trend in decrease of the height of the warts in the omiganan group compared to the vehicle group (p=0.0543).

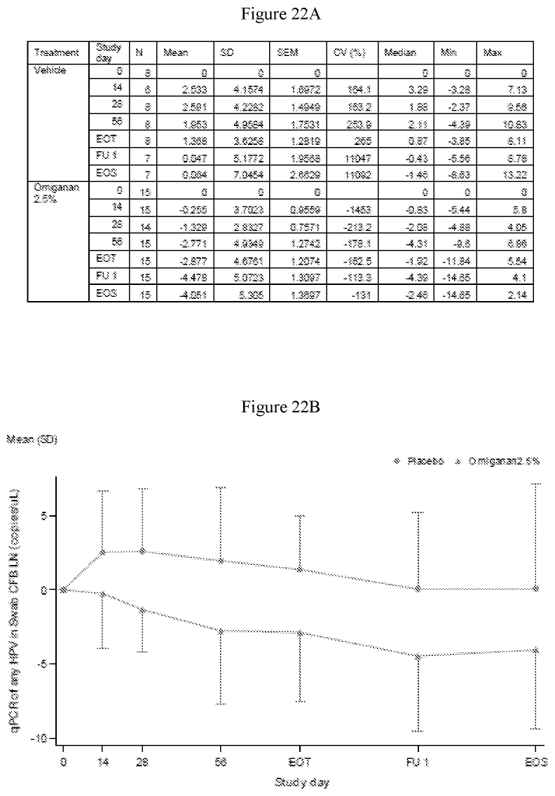

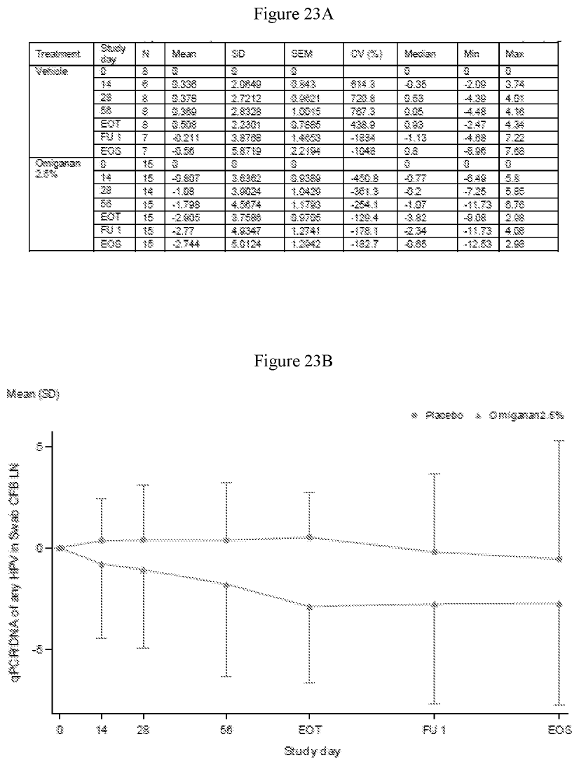

HPV viral load in swabs. Swabs were taken from the target wart (or site where target wart was) at every study visit. A quantitative PCR (qPCR) of the swabs was performed for HPV6 and HPV11 to determine the viral load of the specific HPV. Swab samples were collected at day 0, 14, 28, 56, 84 (EOT), 128 and 168 (EOS). In FIGS. 22A and 22B, the qPCR was expressed as copies/μL and natural log transformed. In FIGS. 23A and 23B, the qPCR was expressed as copies/μL, natural log transformed and also corrected for the amount of DNA in the sample. At EOT there was a statistical significant decrease of HPV6 and HPV11 viral load in the omiganan group compared to the placebo (p=0.0201).

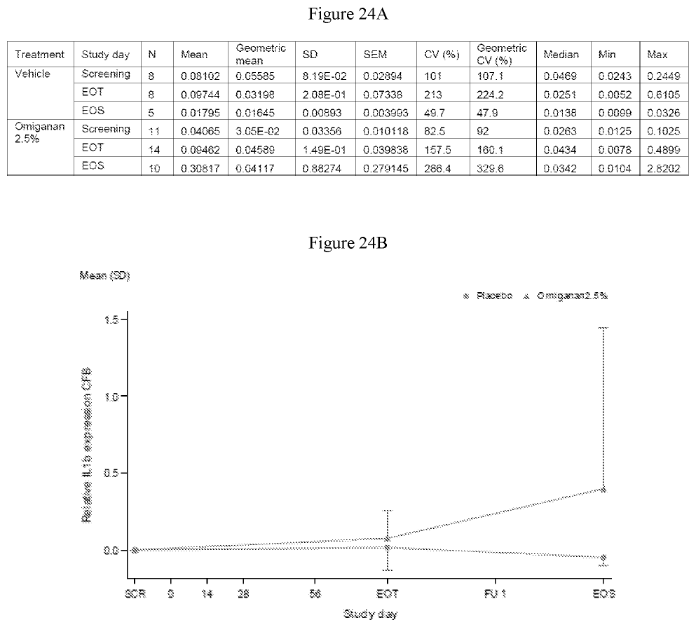

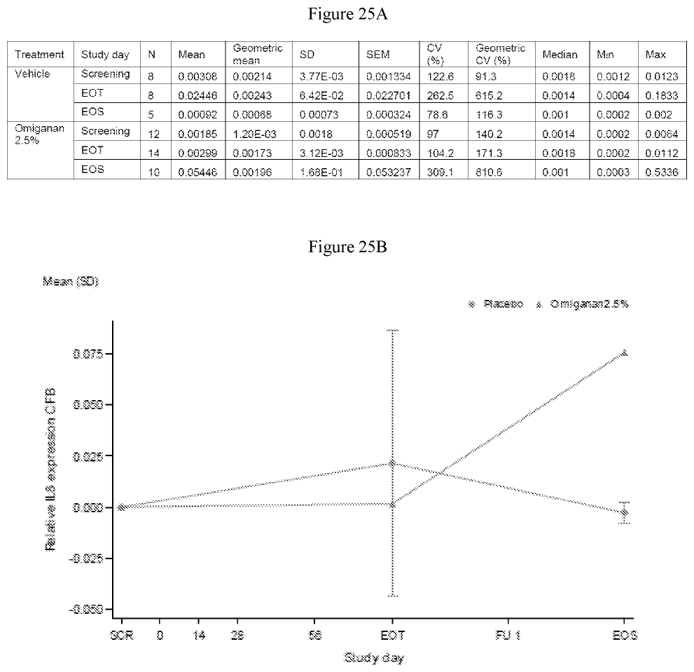

Biomarkers in biopsy. Biopsies were taken from biopsy wart 1 at screening, biopsy wart 2 at EOT and target wart at EOS. Biopsies were only taken when the warts were still visible. A quantitative PCR (qPCR) of the biopsies was performed for the markers IL-1(3, IL-8, IFN-α, and IFN-β. The qPCR was expressed as copies/μL and corrected for the amount of DNA and the percentage of epithelium in the sample. There was no IFN-α and IFN-β detectable in the biopsies. In the omiganan group, the amount of IL-1β and IL-8 increased during the study period, while in the placebo group the amount of these markers remained stable. FIGS. 24A and 24B show the expression of IL-1β and FIGS. 25A and 25B show the expression of IL-8.

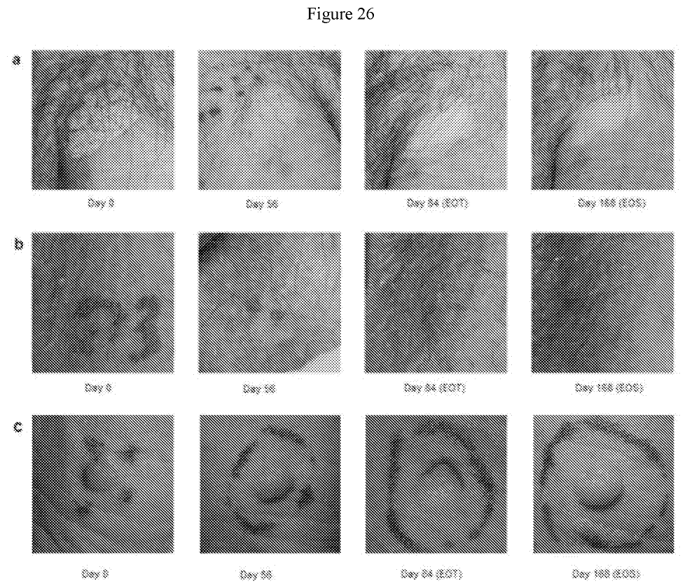

Photography. FIG. 26 shows sets of photographs of clearing anogenital warts. Pre-dose (day 0) the warts are clearly visible in all subjects. Upon treatment, the warts were clearly resolving characterized by visible wart size reduction or even clearance in the two subjects treated with omiganan (panels a and b of FIG. 26). No clearance was seen in the subject treated with placebo (panel c of FIG. 26).

Summary. The results from the study show that omiganan is safe for administration to patients with external anogenital warts. The study demonstrated the pharmacodynamic and clinical activity of the topical application of omiganan in external anogenital warts. Treatment with omiganan showed a significant reduction in volume of the warts and a trend in reduction of the height and wart count with few adverse events supporting a favorable benefit/risk profile. In addition, qPCR data showed a significant reduction in HPV6 and HPV11 viral load at the end of the treatment period along with strong trend toward reduction of viral load of HPV6 in swabs during omiganan treatment.

### Example 5 Modulatory Effects of Omiganan on Ongoing Inflammation in PBMC Cultures

To understand the effect of omiganan on diseases characterized by ongoing chronic inflammation, e.g., ongoing viral infections, the effects of omiganan on already ongoing inflammatory responses were also studied. A series of experiments was thus conducted to investigate: first initiation of innate immune signaling in human PBMCs by a TLR7 trigger, imiquimod, and then addition of omiganan to the cell culture.

Briefly, PBMC primary cultures were prepared from 4 donors. The cultures were incubated with varying doses of imiquimod (50, 10, 2, 0.4, 0.08 μg/mL) for 3 hrs followed by a 21 hr stimulation with varying doses of omiganan (25, 12.5, 6.25 μg/mL) without replacing the culture medium. The readouts for the following cytokine and chemokine release were measured: IL-1β, IL-2, IL-4, IL-6, IL-8 HA, IL-8, IL-12p70, IL-13, IFN-γ, and TNF-α were measured. The cultures were prepared in duplicate.

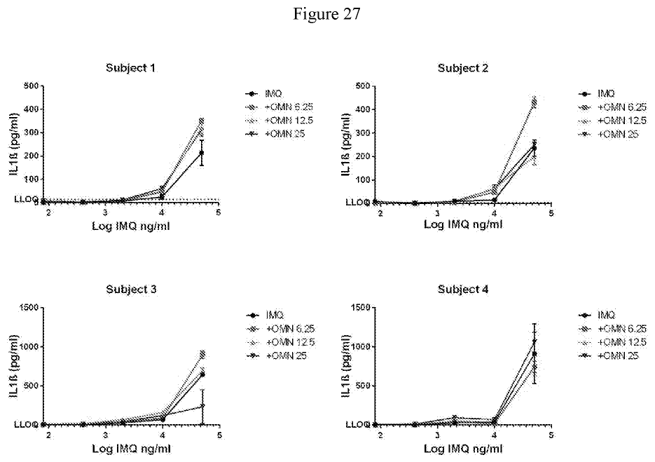

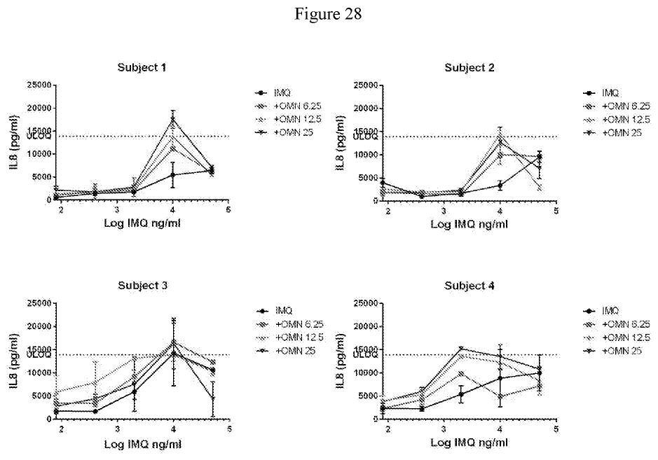

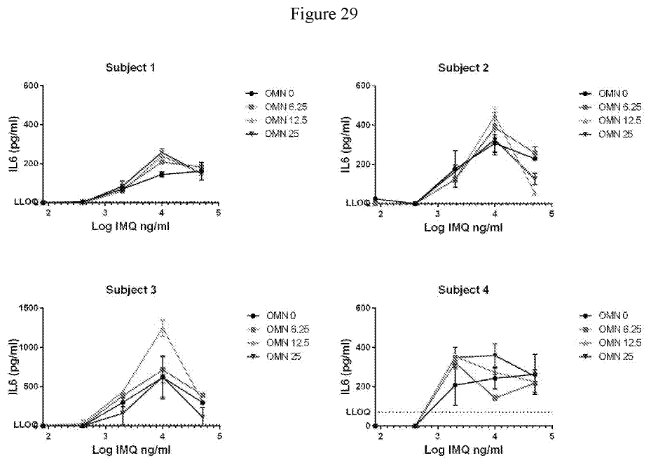

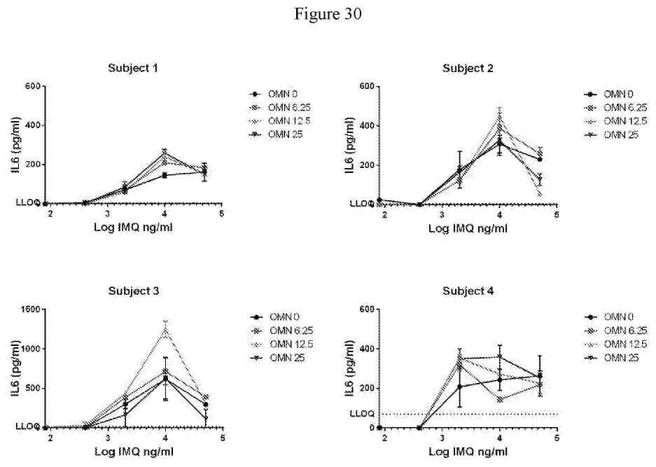

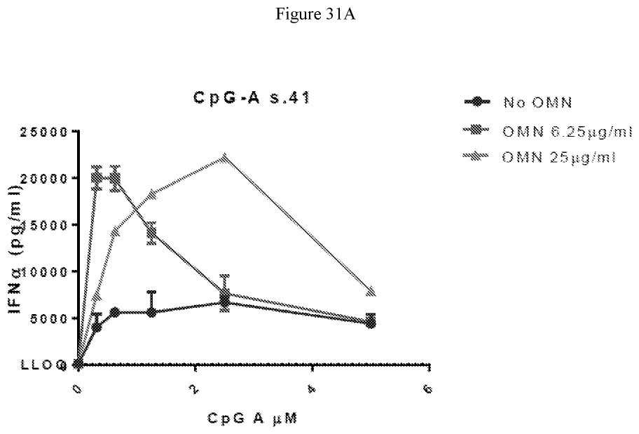

FIGS. 27-30 show the individual immune responses following TLR7 stimulation in the presence and absence of 3 omiganan concentrations. The average cytokine levels for duplicate cultures, plus standard deviation is shown. Data are shown for IL-1β, IL-6, IL-8 and TNF-α. All other cytokine concentrations in the cell culture supernatants were below or around the LLOQ of the assay. Imiquimod alone at concentrations exceeding 0.4 μg/ml induced IL-6, IL-8 and TNF-α release, however concentration exceeding 2 μg/ml were needed for IL-10 release. For IL-6 and TNF-α, the concentration-effect relationship was bell-shaped. Addition of omiganan to the culture medium, after an initial 3 hour incubation with imiquimod, did not significantly modify the IL-1β response (FIG. 27). In contrast, omiganan treatment significantly enhanced the IL-8 response (FIG. 28). In addition, omiganan enhanced the IL-6 and TNF-α responses, although this effect was less obvious (FIGS. 29 and 30).

The treatment sequence (omiganan-trigger versus trigger-omiganan) thus impacts omiganan's effects on TLR7 responses. The induction of IL-8, TNFα, and IL-6, IL1β by omiganan demonstrates the immune-stimulating effects of omiganan and is relevant for its anti-viral effect.

### Example 6 Effects of Omiganan on TLR9-Mediated Interferon Responses in PBMC Cultures

A series of experiments was conducted with various TLR9 triggers to confirm the enhancing effect of omiganan on TLR9-driven interferon responses. To provide more insight into the mechanism by which omiganan may exert this effect, TLR9 triggers with different modes of action were selected: CpG-A, CpG-B, CpG-C (InvivoGen, San Diego, Calif.) and ssDNA. CpG motifs are present in a variety of DNA viruses and CpG signaling is relevant to host defense against viral infections.

Briefly, PBMC primary cultures were prepared from 2 donors. 0.5×10⁶ PBMCs/well/96 well plate were pre-incubated for 30min with either 6.25 or 25 μg/mL of omiganan followed by a 24hr stimulation with varying doses of CpG class A (5, 2.5, 1.25, 0.625, 0.3125 μM); CpG class B (5, 2.5, 1.25, 0.625, 0.3125 μM); CpG class B (5, 2.5, 1.25, 0.625, 0.3125 μM); E. coli ssDNA (5 μg/mL) or PBS (unstimulated). The readouts for IFN-α in cell supernatant were measured by ELISA.

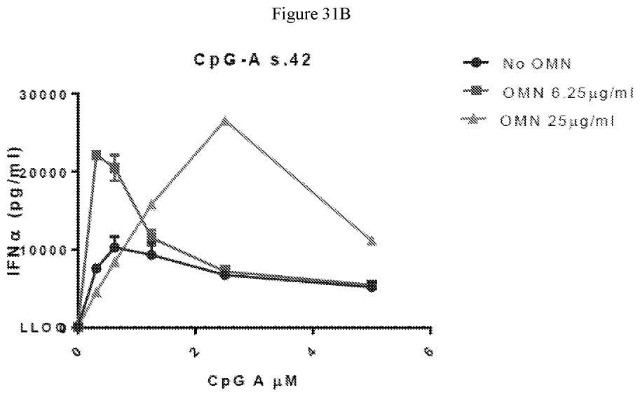

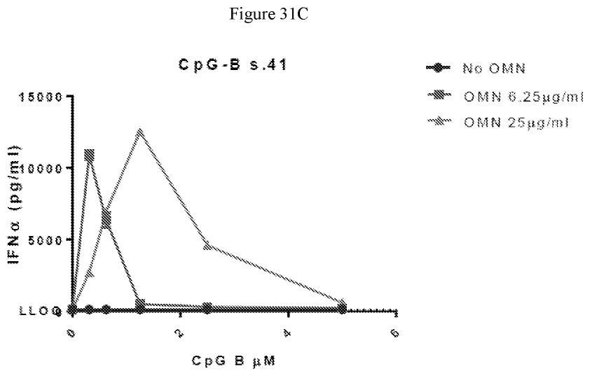

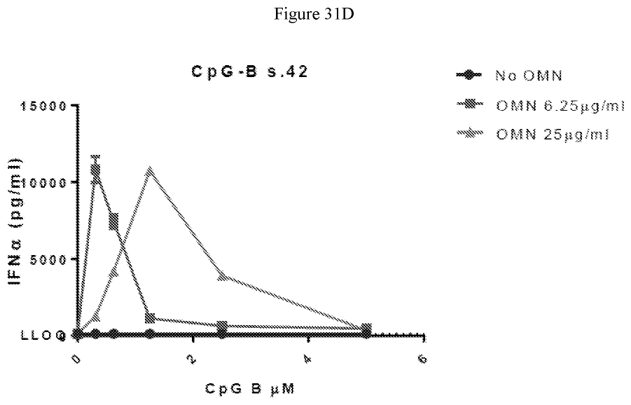

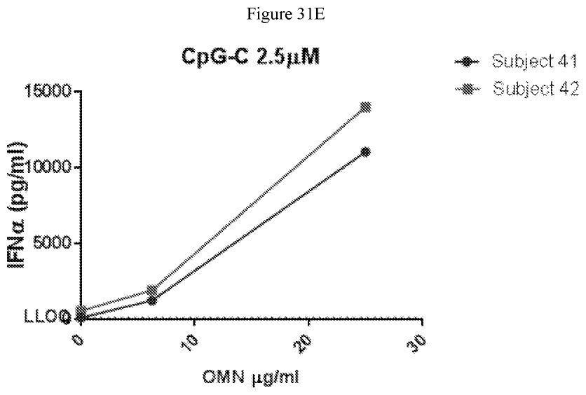

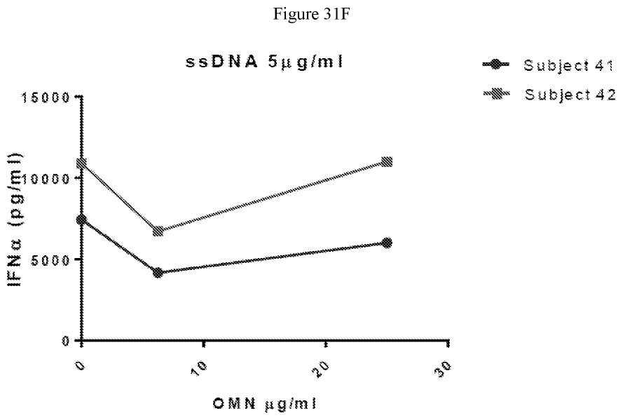

In the absence of omiganan, CpG-A and ssDNA induced significant IFN-α responses, whereas CpG-B and CpG-C did not (FIGS. 31A-31F). Omiganan strongly enhanced CpG-A responses. The CpG-A concentration-response relationship in the presence of omiganan was bell-shaped, with a different optimum for the different omiganan concentrations. Whereas CpG-B alone did not induce IFN-α responses, it did so strongly in the presence of omiganan. Also here, the trigger concentration-response relationship in the presence of omiganan was bell-shaped, with a different optimum for the different omiganan concentrations. Furthermore, omiganan concentration-dependently enhanced CpG-C responses, which on its own did not induce significant cytokine levels. For ssDNA-driven responses, the same omiganan concentration-response relationship was observed as in earlier experiments, with an initial inhibition of cytokine release at low omiganan concentrations, and an enhancement of cytokine release at higher omiganan concentrations.

Thus, omiganan strongly enhanced CpG-A-, CpG-B- and CpG-C-driven IFN-α responses. CpG-B and CpG-C alone did not drive a detectable IFN-α response, but in the presence of omiganan a very strong IFN-α response was observed. Without wishing to be bound by theory, this may be explained by omiganan acting as a carrier for the TLR triggers. For the ssDNA-driven responses, the ssDNA was complexed to Lyovec which serves as carrier enabling cell entry and intracellular transport. Thus the deviating findings for the ssDNA responses compared to the other TLR9 triggers can be explained by omiganan exerting its action by serving as a carrier for single stranded nucleotides.

Without wishing to be bound by theory, thus, the data suggests that omiganan forms a complex with CpG and exerts its interferon response through the interaction of the complex with the PBMCs. Accordingly, these results demonstrate that administration of omiganan in conjunction with viral DNA motifs is useful in methods for treating a viral infection in a subject in need thereof by inducing an interferon response.

### Example 7 Effects of Omiganan on Tumor Growth in Mouse Models

The potential anti-tumor effect of omiganan in vivo was explored by conducting studies in TC-1 mice. Briefly, mice (5 mice per group, 1 tumor per mouse) were inoculated with a TC-1 tumor on day 0 followed by treatment with repeated intratumoral injections of 30 nmol omiganan, 30 nmol of LL-22 (a variant of LL-37), or an equal volume of PBS on days 10, 12, and 14. Tumor growth was measured at days 9, 10, 12, 14, and 16.

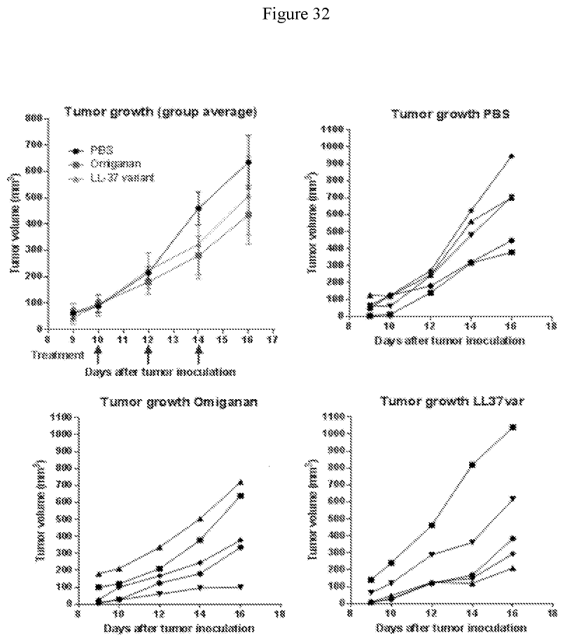

Mice were treated with three intratumoral injections of omiganan, LL-22 (a variant of LL-37), or PBS on day 10, 12 and 14. Tumor growth was measured at day 9 (one day before injection), 10, 12, 14 and day 16 (at sacrifice). A reduction in tumor outgrowth was observed for the omiganan- and LL-37 variant-treated groups compared to PBS-treated mice (FIG. 32).

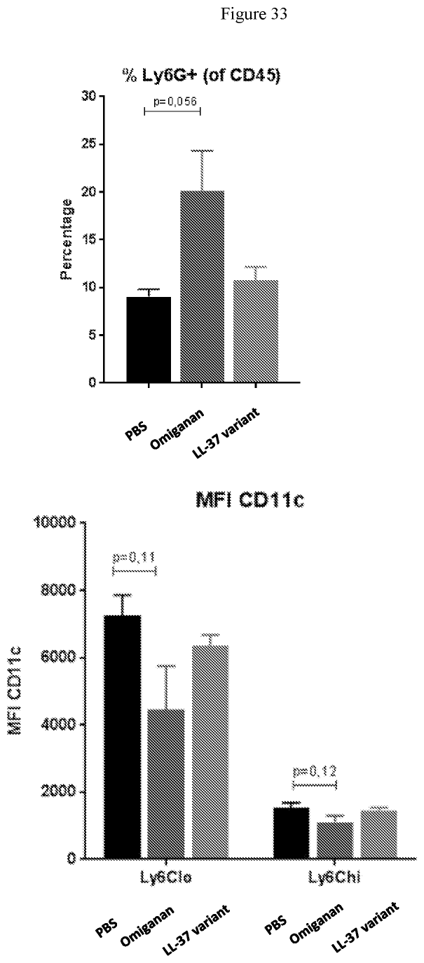

In addition, flow cytometric analyses of common myeloid cell surface markers showed an increase in Ly6G+ cells, meaning an increase in granulocytes in the TC-1 tumor. Further flow cytometric analyses shows that the Ly6C-low macrophages in TC-1 tumors had lower CD11c expression after omiganan treatment (FIG. 33). Although for the LL-37 variant also slower tumor growth was observed, the peptide did not induce the effects on macrophage phenotype as observed for omiganan. Without wishing to be bound by theory, the reduction of CD11c in certain types of tumor macrophages with omiganan indicates that omiganan is having a cell-based adjuvant effect that is related to its anti-tumor activity.

Thus, initial experiments in TC-1 mice show a reduction in tumor outgrowth after repeated injections of omiganan or LL-37 variant compared to PBS treatment. Accordingly, these results demonstrate that administration of omiganan is useful in methods of treating virally-induced tumors in a subject in need thereof. The results further demonstrate that omiganan is useful in methods of inducing a cell-based adjuvant effect in virally-induced cancers.

### Example 8 Omiganan's Effects on Tumor Growth are Dependent on Neutrophils

Mice (6 mice per group, 1 tumor per mouse) were inoculated with a TC-1 tumor on day 0 followed by treatment with repeated intratumoral injections of omiganan (two groups) or an equal volume of PBS (two groups) on days 9, 11, 14, 16, and 18. One of the omiganan treatment groups and one of the PBS control groups were further treated with repeated injections of the anti-neutrophil antibody aLy6G on days 9, 11, 14, 17, and 20. 10, 12, and 14. Tumor growth was measured at days 11, 13, 15, 17, 20, and 22.

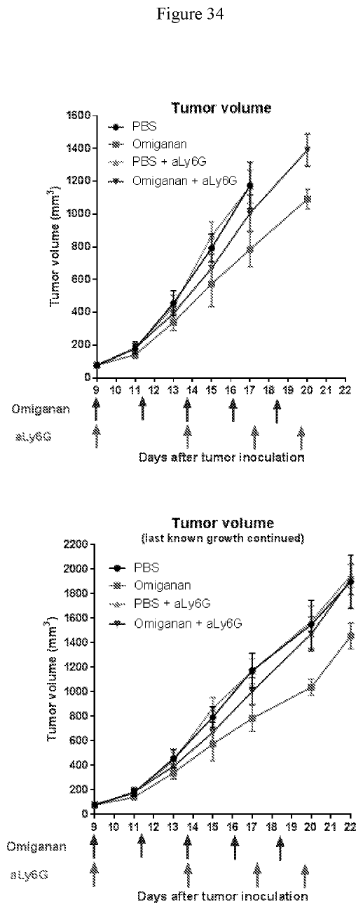

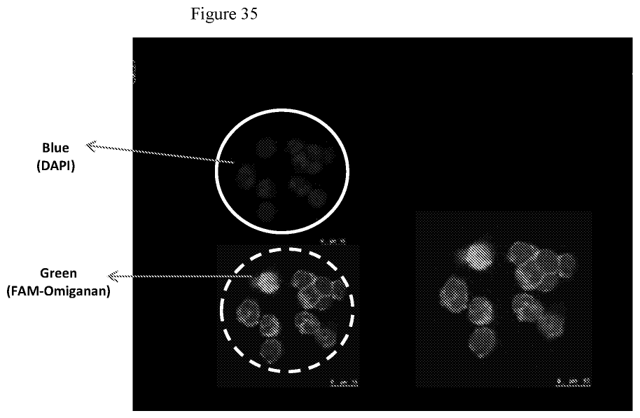

A reduction in tumor outgrowth was observed for the omiganan treated groups compared to PBS-treated mice (FIG. 34). However, this effect was not observed when the omiganan treated group was also treated with aLy6G, indicating that omiganan is exerting its anti-tumor effect via neutrophils.

Accordingly, these results demonstrate that omiganan is useful in methods of inducing a cell-based adjuvant effect in virally-induced cancers.

### Example 9 Omiganan Enters Human PBMCs In Vitro

PBMCs were incubated with FAM labeled omiganan for 10 minutes (25 μg/mL). Confocal microscopy confirmed that that omiganan enters the cells (FIG. 35). The image shows DAPI/FAM overlay and is shown at 100 times magnification+3 times zoom in. The blue indicates DAPI nuclei staining and the green indicates FAM-omiganan.

### Example 10 Effects of Omiganan in Human Skin

To understand the effect of omiganan on viral skin infections, the immunomodulatory effects of topically applied omiganan and the effects of omiganan on imiquimod-induced inflammatory responses of the skin were studied over a 4-day period.

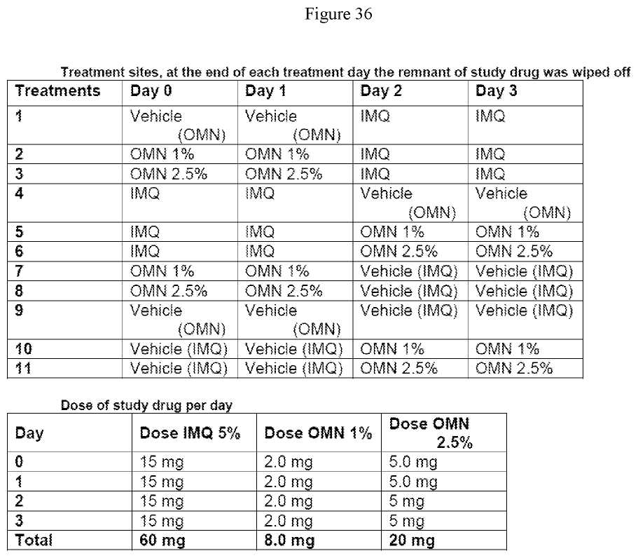

Sixteen (16) healthy subjects participated in the study. The 11 treatments administered are shown in FIG. 36. Pharmacodynamic effects were assessed on Day 1, 2, 3, 4, 5 and 14 (EOS) by clinical assessment of the lesion on-site with visual erythema grading by a blinded investigator, erythema by colorimetry, erythema by multispectral imaging, erythema by 2D photography analysis, perfusion by laser speckle contrast imaging (LSCI), skin surface biomarkers by transdermal analysis patch (TAP) and local biopsies biomarkers.

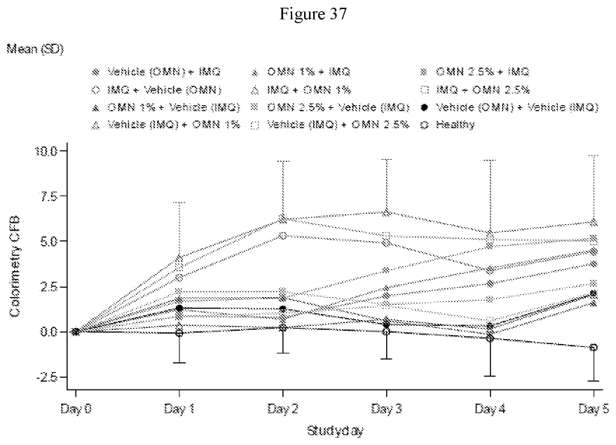

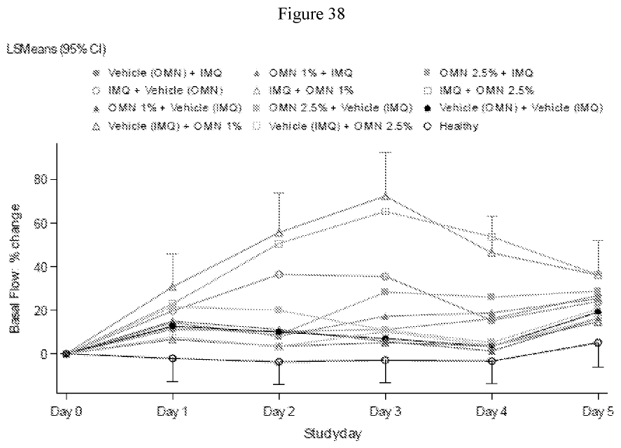

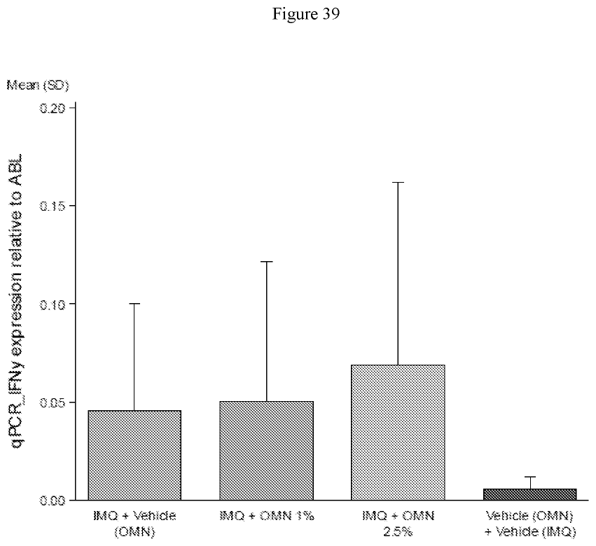

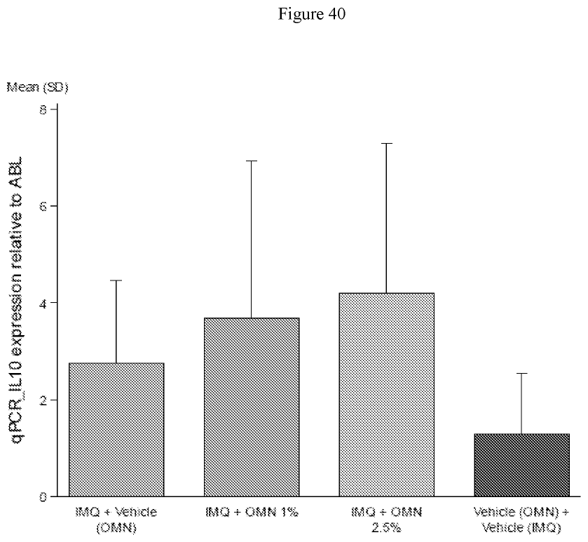

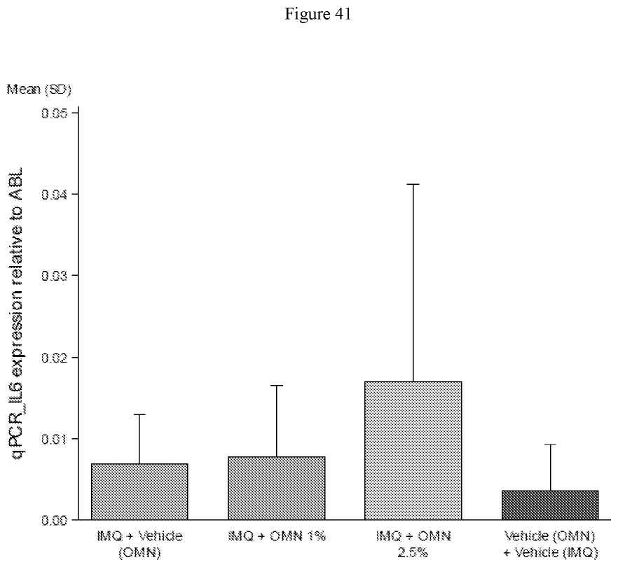

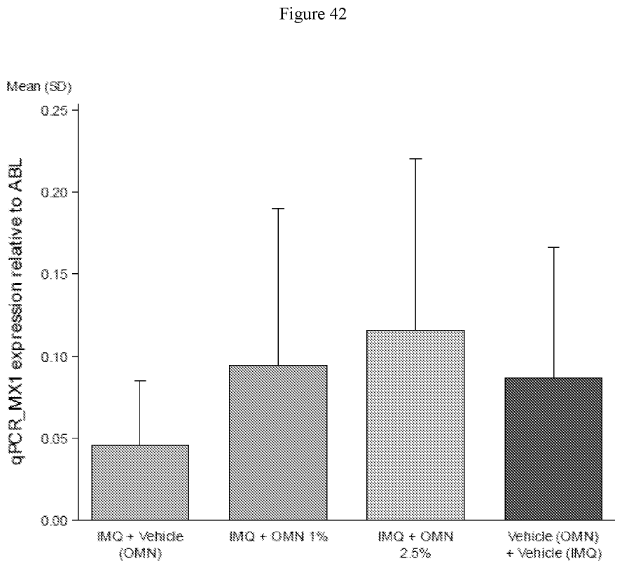

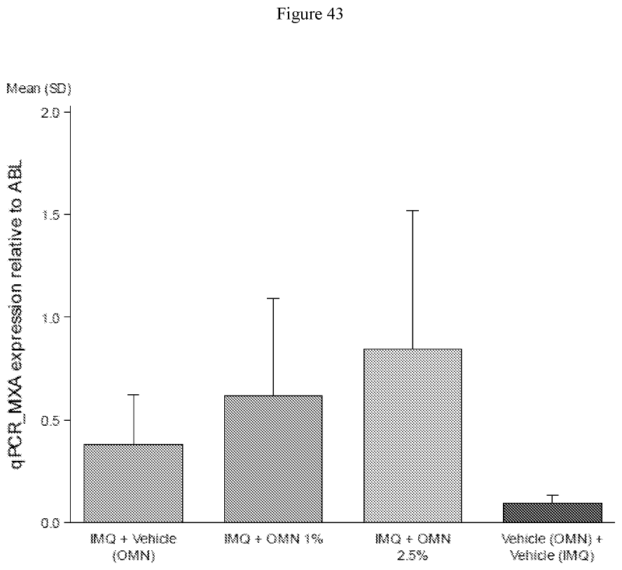

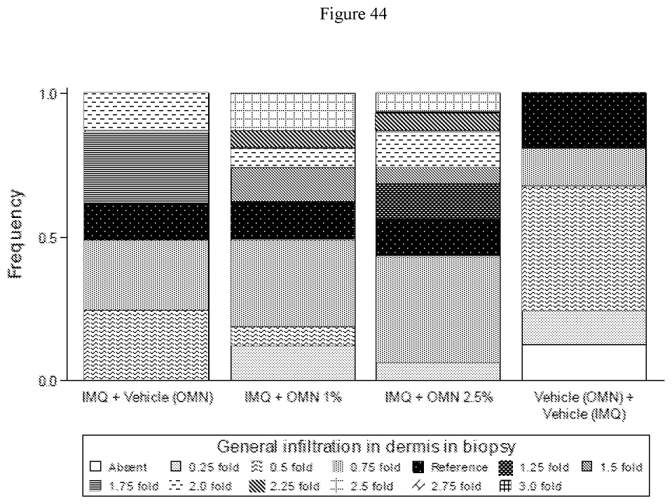

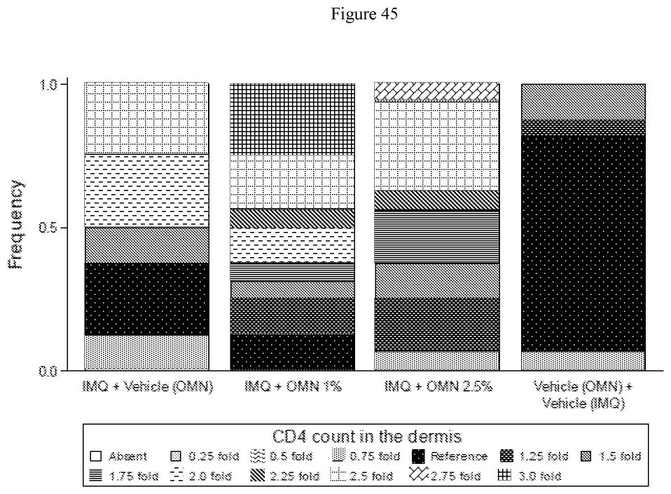

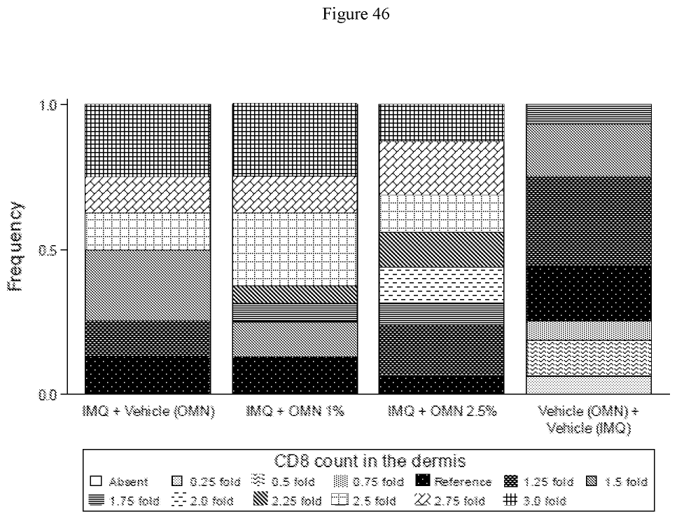

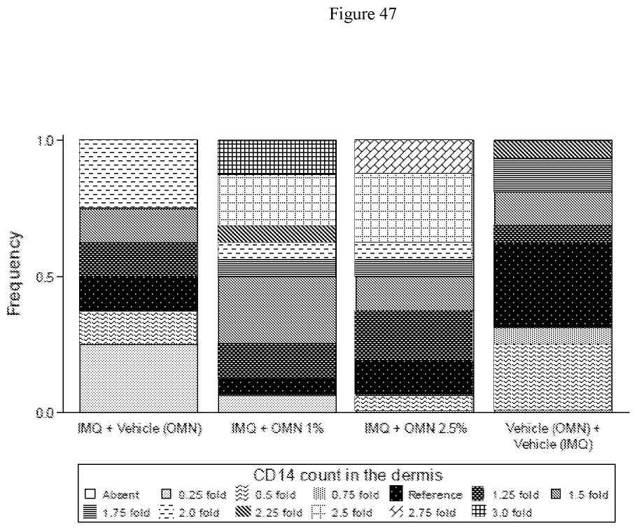

When omiganan was applied first, the effects were similar to those seen with imiquimod. When omiganan was applied after imiquimod application, omiganan enhanced the activity of imiquimod in erythema (FIG. 37) and basal blood flow (FIG. 38). 3 mm skin punch biopsies were collected at day 5 for qPCR mRNA expression, histology and immunohistochemistry. Enhanced inflammation and immune system activation was observed when OMN was applied to 48 hours IMQ primed skin. This was most pronounced for markers IFNγ, IL-10, IL-6, MX1 and MXA (FIGS. 39-43, respectively) indicating an increased anti-viral response. Further, this activity is correlated with cellular infiltrates of CD4+, CD8+ and CD14+ cells (FIGS. 44-47).

OMN enhances the known imiquimod-induced immune effects in human skin in-vivo. This data suggests that combination therapy in virally-induced skin diseases might lead to higher efficacy and lower recurrence rates compared to imiquimod alone.

### Example 11 Anti-HPV Effect of Omiganan is Related to Amount of HPV Genomic Material Available

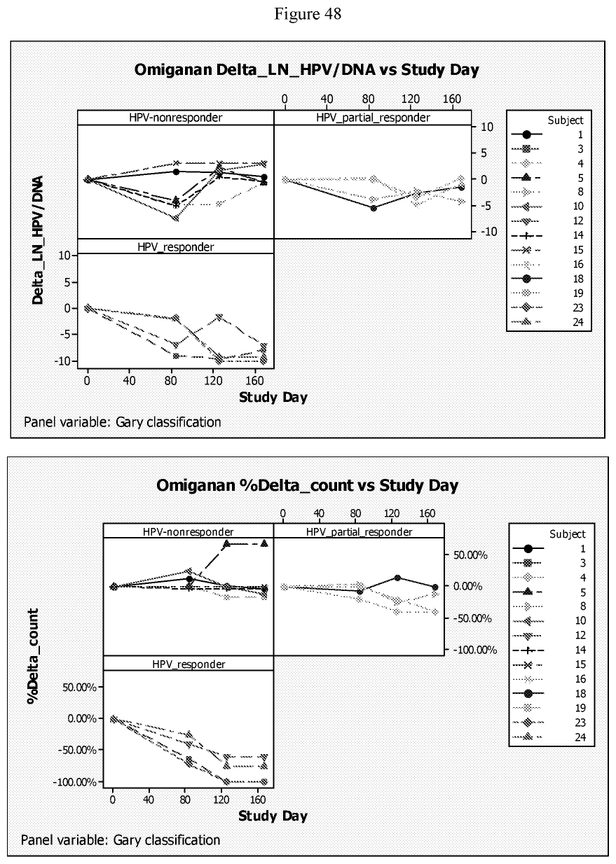

The data from the study described in Example 4 was analyzed post hoc using back biopsy samples to further understand the biologic effects of omiganan. Between subjects who were responders, non-responders, or partial responders to omiganan, it was observed that partial and non-responders often had an initial response during the treatment period which reversed in the follow-up period (FIG. 48). To explore the potential that anti-HPV, anti-lesion effect is related to the pre-treatment levels of HPV genomic material available to theoretically form a complex with omiganan, post-hoc regression analyses were conducted.

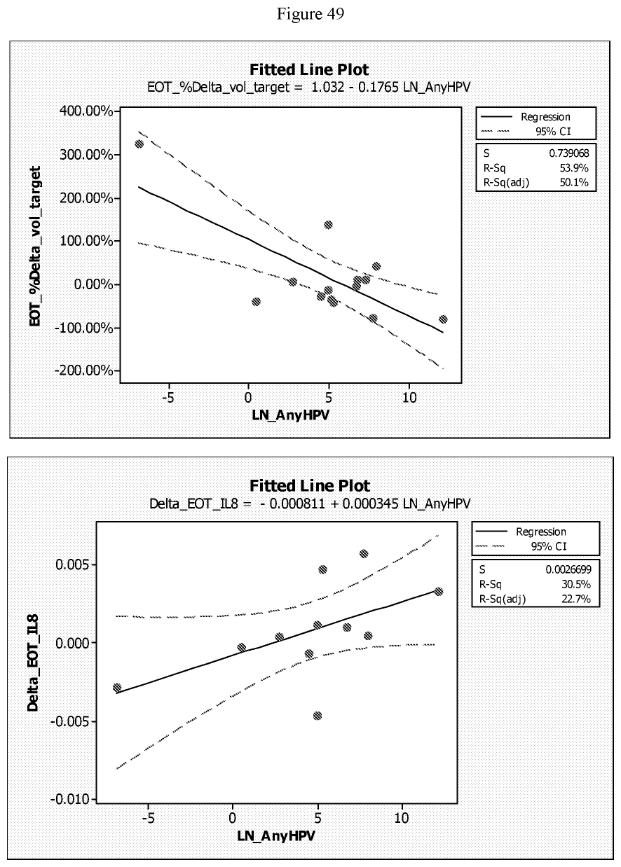

The change in target lesion at end of treatment (EOT) correlated with the HPV DNA load (L1 genomic region) pre-treatment (p=0.003) (FIG. 49). This data suggests that the amount of material available to form a complex with omiganan is related to the anti-HPV anti-lesion response observed upon omiganan treatment. Further, the increase in IL-8 with treatment had a trend relationship with higher pre-treatment HPV DNA (p=0.078) (FIG. 49), thus suggesting a relationship.

Particular embodiments of the invention are set forth in the following numbered paragraphs:
1. A method of enhancing an immune response in a subject in need thereof, said method comprising administering a therapeutically effective amount of a cationic peptide to the subject.
2. The method according to paragraph 1, wherein the cationic peptide is selected from a group consisting of: omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN, and Nt-glucosyl-11J38CN.
3. The method according to paragraph 2, wherein the cationic peptide is omiganan.
4. The method according to paragraph 2, wherein the cationic peptide is LL-37.
5. The method according to any one of paragraphs 1-4, wherein the immune response is an interferon response.
6. The method according to any one of paragraphs 1-5, wherein the immune response is TLR3-mediated.
7. The method according to any one of paragraphs 1-5, wherein the immune response is TLR7-mediated.
8. A method for reducing a viral inflammatory response in a subject in need thereof, said method comprising administering a therapeutically effective amount of a cationic peptide to the subject.
9. The method according to paragraph 8, wherein the cationic peptide is selected from a group consisting of: omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN, and Nt-glucosyl-11J38CN.
10. The method according to paragraph 9, wherein the cationic peptide is omiganan.
11. The method according to any one of paragraphs 8-10, wherein the inflammatory response is cytokine release.
12. The method according to paragraph 11, wherein the cytokine is selected from the group consisting of: TNFα, IL-6, IL1β, and IL-8.
13. The method according to paragraph 12, wherein the cytokine is IL-8.
14. The method according to any one of paragraphs 11-13, wherein the cytokine release is TLR7-mediated.
15. The method according to any one of paragraphs 11-13, wherein the cytokine release is TLR3-mediated.
16. A method for treating viral infection in a subject in need thereof, said method comprising administering to the subject a therapeutically effective amount of a cationic peptide, wherein the therapeutically effective amount is sufficient to deliver a cationic peptide to the site of viral infection at a concentration of 5-25 μg/mL.
17. A method for reducing viral load in a subject in need thereof, said method comprising administering to the subject a therapeutically effective amount of a cationic peptide, wherein the therapeutically effective amount is sufficient to deliver a cationic peptide to the site of viral infection a concentration of 5-25 μg/mL.
18. A method for preventing a viral infection in a subject in need thereof, said method comprising administering a prophylactically effective amount of a cationic peptide to the subject, wherein the prophylactically effective amount is sufficient to deliver a cationic peptide to a potential site of viral infection at a concentration of 5-25 μg/mL.
19. The method according to any one of paragraphs 16-18, wherein the cationic peptide is selected from the group consisting of: omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN, and Nt-glucosyl-11J38CN.
20. The method according to paragraph 19, wherein the cationic peptide is omiganan.
21. The method according to paragraph 19, wherein the cationic peptide is LL-37.
22. The method according to any one of paragraphs 8-21, wherein the plasma or local concentration of the cationic peptide is higher than the IC₅₀ of the virus causing the viral infection 12 hours following administration of the cationic peptide.
23. The method according to any one of paragraphs 1-22, wherein the cationic peptide is administered topically or parenterally.
24. The method according to paragraph 23, wherein the administration is epicutaneous, by inhalation, intranasal, by an enema, by eye drops, by ear drops, or through a mucous membrane.
25. The method according to paragraph 23 or 24, wherein the cationic peptide is administered topically in a composition comprising a solvent and a viscosity-increasing agent.
26. The method according to paragraph 25, wherein the solvent is selected from the group consisting of: water, glycerin, propylene glycol, mineral oil, isopropanol, ethanol, methanol, and a combination thereof.
27. The method according to paragraph 25, wherein the solvent is a combination of water and glycerin.
28. The method according to any one of paragraphs 25-27, wherein the viscosity-increasing agent is selected from the group consisting of: carbomer homopolymer, dextran, polyvinylpyrrolidone, hydroxyethyl cellulose, hydroxypropyl methylcellulose, and a combination thereof.
29. The method according to paragraph 28, wherein the viscosity-increasing agent is hydroxyethyl cellulose.
30. The method according to any one of paragraphs 25-29, wherein the composition further comprises a buffering agent.
31. The method according to paragraph 30, wherein the buffering agent comprises a monocarboxylate or a dicarboxylate.
32. The method according to paragraph 30, wherein the buffering agent comprises acetate, benzoate, fumarate, lactate, malonate, sorbate, succinate, or tartrate.
33. The method according to paragraph 32, wherein the buffering agent is benzoate.
34. The method of any one of paragraphs 25-33, wherein the composition has a pH from about 3 to about 8.
35. The method according to any one of paragraph 25-34, wherein the composition further comprises a humectant.
36. The method according to paragraph 35, wherein the humectant is sorbitol or glycerol.
37. The method according to any one of paragraphs 25-36, wherein the composition further comprises a preservative.
38. The method according to paragraph 37, wherein the preservative is selected from the group consisting of: benzoic acid, benzyl alcohol, phenoxyethanol, methylparaben, propylparaben, and a combination thereof.
39. The method according to any one of paragraphs 25-38, wherein the composition further comprises an additional antiviral agent.
40. The method according to paragraph 39, wherein the additional antiviral agent wherein the antiviral agent is selected from the group consisting of: amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha, and edoxudine.
41. The method according to any one of paragraphs 25-40, wherein the composition is a gel.
42. The method according to paragraph 23, wherein the parenteral administration is subcutaneous, intravenous, intramuscular, intraarterial, intraabdominal, intraperitoneal, intraarticular, intraocular or retrobulbar, intraaural, intrathecal, intracavitary, intracelial, intraspinal, intrapulmonary or transpulmonary, intrasynovial, or intraurethral injection or infusion.
43. The method according to any one of paragraphs 8-42, wherein the viral infection is caused by a virus selected from the group consisting of: coronavirus, coxsackievirus, cytomegalovirus, echovirus, enterovirus, Epstein-Barr virus, influenza virus, hepatotropic viruses, HIV, HPV, herpes simplex virus, pox virus, norovirus, rabies virus, rhinovirus, rotavirus, RSV, Varicella zoster virus, parvovirus, and West Nile virus.
44. The method according to paragraph 43, wherein the viral infection is caused by HPV.
45. The method according to paragraph 44, wherein the HPV is HPVS.
46. The method according to any one of paragraphs 16-43, wherein the method further comprises administering an additional antiviral agent.
47. The method according to paragraph 46, wherein the antiviral agent is amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha, or edoxudine.
48. The method according to paragraph 46 or 47, wherein the cationic peptide and additional antiviral agent are in the same composition.
49. The method according to paragraph 46 or 47, wherein the cationic peptide and additional antiviral agent are in different compositions.
50. The method according to any one of paragraphs 46-49, wherein the cationic peptide and additional antiviral agent are administered simultaneously.
51. The method according to any one of paragraphs 46, 47 or 49, wherein the cationic peptide and additional antiviral agent are administered sequentially.
52. The method according to any one of paragraphs 16-43, wherein the method further comprises administering an additional viral uptake inhibitor.
53. The method according to paragraph 52, wherein the additional viral uptake inhibitor is furin inhibitor I, amantadine, rimantadine, interferon, glycyrrhizin, or pentafuside.
54. The method according to paragraph 52 or 53, wherein the cationic peptide and additional viral uptake inhibitor are in the same composition.
55. The method according to paragraph 52 or 53, wherein the cationic peptide and additional viral uptake inhibitor are in different compositions.
56. The method according to any one of paragraphs 52-55, wherein the cationic peptide and additional viral uptake inhibitor are administered simultaneously.
57. The method according to any one of paragraphs 52, 53 or 55, wherein the cationic peptide and additional viral uptake inhibitor are administered sequentially.
58. A method for reducing inflammatory response in a subject in need thereof, said method comprising administering to the subject a therapeutically effective amount of a cationic peptide, wherein the therapeutically effective amount is sufficient to deliver a cationic peptide to the site of inflammatory response at a concentration of 5-25 μg/mL.
59. The method according to paragraph 58, wherein the cationic peptide is selected from the group consisting of: omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN, and Nt-glucosyl-11J38CN.
60. The method according to paragraph 59, wherein the cationic peptide is omiganan.
61. The method according to paragraph 59, wherein the cationic peptide is LL-37.
62. The method according to any one of paragraphs 58-61, wherein the cationic peptide is administered topically or parenterally.
63. The method according to paragraph 62, wherein the administration is epicutaneous, by inhalation, intranasal, by an enema, by eye drops, by ear drops, or through a mucous membrane.
64. The method according to paragraph 62 or 63, wherein the cationic peptide is administered topically in a composition comprising a solvent and a viscosity-increasing agent.
65. The method according to paragraph 64, wherein the solvent is selected from the group consisting of: water, glycerin, propylene glycol, mineral oil, isopropanol, ethanol, methanol, and a combination thereof.
66. The method according to paragraph 64, wherein the solvent is a combination of water and glycerin.
67. The method according to any one of paragraphs 64-66, wherein the viscosity-increasing agent is selected from the group consisting of: carbomer homopolymer, dextran, polyvinylpyrrolidone, hydroxyethyl cellulose, hydroxypropyl methylcellulose, and a combination thereof.
68. The method according to paragraph 67, wherein the viscosity-increasing agent is hydroxyethyl cellulose.
69. The method according to any one of paragraphs 64-68, wherein the composition further comprises a buffering agent.
70. The method according to paragraph 69, wherein the buffering agent comprises a monocarboxylate or a dicarboxylate.
71. The method according to paragraph 69, wherein the buffering agent comprises acetate, benzoate, fumarate, lactate, malonate, sorbate, succinate, or tartrate.
72. The method according to paragraph 71, wherein the buffering agent is benzoate.
73. The method of any one of paragraphs 64-72, wherein the composition has a pH from about 3 to about 8.
74. The method according to any one of paragraph 64-73, wherein the composition further comprises a humectant.
75. The method according to paragraph 74, wherein the humectant is sorbitol or glycerol.
76. The method according to any one of paragraphs 64-75, wherein the composition further comprises a preservative.
77. The method according to paragraph 76, wherein the preservative is selected from the group consisting of: benzoic acid, benzyl alcohol, phenoxyethanol, methylparaben, propylparaben, and a combination thereof
78. The method according to any one of paragraphs 64-77, wherein the composition is a gel.
79. The method according to paragraph 62, wherein the parenteral administration is subcutaneous, intravenous, intramuscular, intraarterial, intraabdominal, intraperitoneal, intraarticular, intraocular or retrobulbar, intraaural, intrathecal, intracavitary, intracelial, intraspinal, intrapulmonary or transpulmonary, intrasynovial, or intraurethral injection or infusion.
80. A method of treating or alleviating the symptoms of anogenital warts caused by an HPV infection in a subject in need thereof, comprising administering to the subject a therapeutically effective amount of a cationic peptide.
81. The method according to paragraph 80, wherein the therapeutically effective amount is sufficient to deliver the cationic peptide to the site of the anogenital warts at a concentration of 5-25 μg/mL.
82. The method according to paragraph 80 or 81, wherein the HPV is HPV6 or HPV11.
83. The method according to any one of paragraphs 80-82, wherein the administration of the cationic peptide reduces one or more of wart count, wart height, or wart volume.
84. The method according to any one of paragraphs 80-83, wherein the administration of the cationic peptide reduces viral load at the site of the anogenital warts.
85. A method of treating a virally-induced cancer in a subject in need thereof, comprising administering to the subject a therapeutically effective amount of a cationic peptide.
86. The method according to paragraph 85, wherein the cationic peptide reduces tumor growth.
87. The method according to paragraph 85-86, wherein the cationic peptide is complexed with a viral polynucleotide sequence.
88. The method according to paragraph 87, wherein the viral polynucleotide sequence is a viral DNA.
89. The method according to paragraph 88, wherein the viral DNA is a sequence comprising CpG dinucleotides (CpG motif).
90. The method according to any one of paragraphs 85-89, wherein the virally-induced cancer is caused by Human Papillomavirus (HPV), Epstein-Barr virus (EBV), hepatitis B virus (HBV), hepatitis C virus (HCV), Human T-lymphotropic virus 1 (HTLV 1), Kaposi's sarcoma-associated herpesvirus (KSHV or HHV8), Merkel cell polyoma virus (MCV), or Human cytomegalovirus (CMV or HHV-5).
91. The method according to any one of paragraphs 80-90, wherein the cationic peptide is selected from the group consisting of: omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN, and Nt-glucosyl-11J38CN.
92. The method according to paragraph 91, wherein the cationic peptide is omiganan or LL-37.
93. A cationic peptide complex comprising a cationic peptide and a viral polynucleotide sequence.
94. The cationic peptide complex of paragraph 93, wherein the cationic peptide is selected from the group consisting of: omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN, and Nt-glucosyl-11J38CN.
95. The cationic peptide complex according to paragraph 94, wherein the cationic peptide is omiganan or LL-37.
96. The cationic peptide complex according to any one of paragraphs 93-95, wherein the viral polynucleotide sequence is a viral DNA.
97. The cationic peptide complex according to paragraph 96, wherein the viral DNA is a sequence comprising CpG dinucleotides (CpG motif).
98. Use of a cationic peptide in the preparation of a medicament for enhancing an immune response in a subject.
99. Use of a cationic peptide in the preparation of a medicament for modulating a viral inflammatory response in a subject.
100. Use of a cationic peptide in the preparation of a medicament for treating viral infection in a subject, wherein the medicament comprises an amount of cationic peptide sufficient to deliver the cationic peptide to the site of viral infection at a concentration of 5-25 μg/mL.
101. Use of a cationic peptide in the preparation of a medicament for reducing viral load in a subject, wherein the medicament comprises an amount of cationic peptide sufficient to deliver the cationic peptide to the site of viral infection a concentration of 5-25 μg/mL.
102. Use of a cationic peptide in the preparation of a medicament for preventing a viral infection in a subject, wherein the medicament comprises an amount of cationic peptide sufficient to deliver the cationic peptide to a potential site of viral infection at a concentration of 5-25 μg/mL.
103. Use of a cationic peptide in the preparation of a medicament for reducing inflammatory response in a subject, wherein the medicament comprises an amount of cationic peptide sufficient to deliver the cationic peptide to the site of inflammatory response at a concentration of 5-25 μg/mL.
104. Use of a cationic peptide in the preparation of a medicament for treating or alleviating the symptoms of anogenital warts caused by an HPV infection in a subject.
105. Use of a cationic peptide in the preparation of a medicament for treating a virally-induced cancer in a subject.
106. The use according to any one of paragraphs 98-105, wherein the cationic peptide is selected from a group consisting of: omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN, and Nt-glucosyl-11J38CN.
107. The use according to paragraph 106, wherein the cationic peptide is omiganan or LL-37.
108. A cationic peptide for use in enhancing an immune response in a subject.
109. A cationic peptide for use in modulating a viral inflammatory response in a subject.
110. A cationic peptide for use in treating viral infection in a subject, wherein the medicament comprises an amount of cationic peptide sufficient to deliver the cationic peptide to the site of viral infection at a concentration of 5-25 μg/mL.
111. A cationic peptide for use in reducing viral load in a subject, wherein the medicament comprises an amount of cationic peptide sufficient to deliver the cationic peptide to the site of viral infection a concentration of 5-25 μg/mL.

1.-80. (canceled)

81. A method of enhancing an immune response in a subject in need thereof, said method comprising administering a therapeutically effective amount of a cationic peptide to the subj ect.

82. A method of treating or preventing viral infection or a virally-induced cancer in a subject in need thereof, said method comprising administering to the subject a therapeutically or prophylactically effective amount of a cationic peptide.

83. The method according to claim 82, wherein the cationic peptide is selected from the group consisting of: omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN, and Nt-glucosyl-11J38CN.

84. The method according to claim 83, wherein the cationic peptide is omiganan or LL-37.

85. The method according to claim 82, wherein the method further comprises administering an additional antiviral agent or an additional immune modulator, wherein the additional antiviral agent is selected from the group consisting of amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha and edoxudine, and wherein the additional immune modulator is selected from the group consisting of imiquimod, polyI:C and CpG DNA.

86. The method according to claim 82, for treating or preventing the viral infection.

87. The method according to claim 86, wherein the therapeutically or prophylactically effective amount is sufficient to deliver the cationic peptide to the site of viral infection at a concentration of 5-25 μg/mL.

88. The method according to claim 86, wherein the viral infection is caused by a virus selected from the group consisting of: coronavirus, coxsackievirus, cytomegalovirus, echovirus, enterovirus, Epstein-Barr virus (EBV), influenza virus, hepatotropic viruses, human immunodeficiency virus (HIV), human papillomavirus (HPV), herpes simplex virus (HSV), pox virus, norovirus, rabies virus, rhinovirus, rotavirus, Rous sarcoma virus (RSV), Varicella zoster virus, parvovirus, and West Nile virus.

89. The method according to claim 88, wherein the viral infection is caused by HPV.

90. The method according to claim 89, wherein the HPV is HPV5, HPV6 or HPV11.

91. The method according to claim 90, wherein the HPV is HPV5.

92. The method according to claim 89, for treating or alleviating the symptoms of anogenital warts caused by an HPV infection.

93. The method according to claim 86, wherein the viral infection is caused by HPV and wherein the cationic peptide is selected from the group consisting of: omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN, and Nt-glucosyl-11J38CN.

94. The method according to claim 93, wherein the cationic peptide is omiganan or LL-37.

95. The method according to claim 93, wherein the HPV is HPV5, HPV6 or HPV11 and wherein the cationic peptide is omiganan or LL-37.

96. The method according to claim 93, for treating or alleviating the symptoms of anogenital warts caused by an HPV infection, wherein the cationic peptide is administered topically and wherein the cationic peptide is omiganan or LL-37.

97. The method according to claim 82, for treating the virally-induced cancer.

98. The method according to claim 97, wherein the virally-induced cancer is caused by HPV, EBV, hepatitis B virus (HBV), hepatitis C virus (HCV), Human T-lymphotropic virus 1 (HTLV 1), Kaposi's sarcoma-associated herpesvirus (KSHV or HHV8), Merkel cell polyoma virus (MCV), or Human cytomegalovirus (CMV or HHV-5).

99. The method according to claim 98, wherein the cationic peptide is selected from the group consisting of: omiganan, LL-37, 11B32CN, 11B36CN, 11E3CN, 11F4CN, 11F5CN, 11F12CN, 11F17CN, 11F50CN, 11F56CN, 11F63CN, 11F64CN, 11F66CN, 11F67CN, 11F68CN, 11F93CN, 11G27CN, 11J02CN, 11J02ACN, 11J30CN, 11J36CN, 11J58CN, 11J67CN, 11J68CN, Nt-acryloyl-11B7CN, Nt-glucosyl-11J36CN, and Nt-glucosyl-11J38CN.

100. The method according to claim 99, wherein the cationic peptide is omiganan or LL-37.
